(19) United States
(12) Patent Application Publication
(10) Pub. No.: US 2026/0347490 A1
(43) Pub. Date: Nov. 5, 2026

(54) TNFR2 ANTIBODIES AND METHODS OF USING THE SAME

(71) Applicant: Yale University, New Haven, CT (US)

(72) Inventor: Raymond Johnson, New Haven, CT (US)

(21) Appl. No.: 18/997,251

(22) PCT Filed: Aug. 1, 2023
(86) PCT No.: PCT/US2023/071404

§ 371 Date: Jan. 21, 2025

Related U.S. Application Data
(60) Provisional application No. 63/394,057, filed on Aug. 1, 2022.
(60) Provisional application No. 63/423,223, filed on Nov. 7, 2022.

(51) Int. Cl. C07K 16/28; G01N 33/68

(52) U.S. Cl. CPC C07K 16/2878; G01N 33/6854; C07K 2317/24; C07K 2317/565; G01N 2333/70578; G01N 2800/328; G01N 2800/56

(57) ABSTRACT

Described herein are novel antibodies recognizing extracellular portion of TNFR2 and soluble TNFR2 (sTNFR2) and nucleic acids encoding the same. The antibodies recognizing different epitopes on TNFR2/sTNFR2, and are able to quantify sTNFR2 in samples as capture-detection pairs with high sensitivity and specificity. Also described here are methods of quantifying sTNFR2 and methods of diagnosing, and/or treating, ameliorating, and/or preventing Kawasaki disease and Kawasaki-like syndrome, as well as methods of treating, ameliorating and/or preventing inflammatory diseases or disorders caused by or involves overactive TNFα-TNFR2 signaling.

### CROSS-REFERENCE TO RELATED APPLICATIONS

The present application claims priority under 35 U.S.C. § 119(e) to U.S. Provisional Patent Application No. 63/394,057, filed Aug. 1, 2022 and U.S. Provisional Patent Application No. 63/423,223, filed Nov. 7, 2022, both of which are incorporated herein by reference in its entirety.

### STATEMENT REGARDING FEDERALLY SPONSORED RESEARCH

This invention was made with government support under R41HD093473 awarded by the National Institutes of Health (NIH). The government has certain rights in the invention.

### SEQUENCE LISTING

The XML file named “047162-7390WO1 (02037)_Seq Listing.xml” created on Jul. 19, 2023, comprising 80.1 Kbytes, is hereby incorporated by reference in its entirety.

### BACKGROUND

Altered levels of soluble TNFR2 (sTNFR2) in serum and other body fluids have been recognized as a predictive factor of various diseases and disorders, including but not limited to sepsis, infectious diseases, hepatitis, organ fibrosis, cancer development, and other diseases and disorders involving inflammation.

Accurate quantifications of sTNFR2, however; have been challenging due to the lack of sTNFR2 antibodies suitable for this purpose. For example, Clinical Laboratory Improvement Amendments (CLIA) compliant ELISA-type testing requires a reproducible reagent, which normally relates to a monoclonal antibody pair that are specific for sTNFR2 and in tandem can capture and detect the sTNFR2. However, there are no functional sTNFR2 antibody pairs available.

Therefore, there is a need for novel sTNFR2 antibodies that are able to detect and quantify sTNFR2, and/or are useful for diagnosing, and/or treating, ameliorating, and/or preventing diseases or disorders involving altered sTNFR2 levels. The present invention addresses these needs.

### SUMMARY

In some aspects, the present invention is directed to the following non-limiting embodiments:

In some aspects, the present invention is directed to an antibody.

In some embodiments, the antibody comprises a heavy chain polypeptide and a light chain polypeptide.

In some embodiments, the heavy chain polypeptide comprises a CDR1 comprising an amino acid sequence of SEQ ID NO:1; a CDR2 comprising an amino acid sequence of SEQ ID NO: 2; and a CDR3 comprising an amino acid sequence of SEQ ID NO:3. In some embodiments, the heavy chain polypeptide comprises a CDR1 comprising an amino acid sequence of SEQ ID NO:7; a CDR2 comprising an amino acid sequence of SEQ ID NO:8; and a CDR3 comprising an amino acid sequence of SEQ ID NO:9. In some embodiments, the heavy chain polypeptide comprises a CDR1 comprising an amino acid sequence of SEQ ID NO: 13; a CDR2 comprising an amino acid sequence of SEQ ID NO:14; and a CDR3 comprising an amino acid sequence of SEQ ID NO:15. In some embodiments, the heavy chain polypeptide comprises a CDR1 comprising an amino acid sequence of SEQ ID NO:18; a CDR2 comprising an amino acid sequence of SEQ ID NO: 19; and a CDR3 comprising an amino acid sequence of SEQ ID NO:20. In some embodiments, the heavy chain polypeptide comprises a CDR1 comprising an amino acid sequence of SEQ ID NO:23; a CDR2 comprising an amino acid sequence of SEQ ID NO:24; and a CDR3 comprising an amino acid sequence of SEQ ID NO:25.

In some embodiments, the light chain polypeptide comprises a CDR1 comprising an amino acid sequence of SEQ ID NO:4; a CDR2 comprising an amino acid sequence of SEQ ID NO: 5; and a CDR3 comprising an amino acid sequence of SEQ ID NO:6. In some embodiments, the light chain polypeptide comprises a CDR1 comprising an amino acid sequence of SEQ ID NO: 10; a CDR2 comprising an amino acid sequence of SEQ ID NO:11; and a CDR3 comprising an amino acid sequence of SEQ ID NO: 12. In some embodiments, the light chain polypeptide comprises a CDR1 comprising an amino acid sequence of SEQ ID NO: 4; a CDR2 comprising an amino acid sequence of SEQ ID NO:16; and a CDR3 comprising an amino acid sequence of SEQ ID NO:17. In some embodiments, the light chain polypeptide comprises a CDR1 comprising an amino acid sequence of SEQ ID NO:4; a CDR2 comprising an amino acid sequence of SEQ ID NO:21; and a CDR3 comprising an amino acid sequence of SEQ ID NO:22. In some embodiments, the light chain polypeptide comprises a CDR1 comprising an amino acid sequence of SEQ ID NO:26; a CDR2 comprising an amino acid sequence of SEQ ID NO:27; and a CDR3 comprising an amino acid sequence of SEQ ID NO:28.

In some embodiments, the antibody comprises a heavy chain polypeptide comprising a CDR1 comprising an amino acid sequence of SEQ ID NO:1, a CDR2 comprising an amino acid sequence of SEQ ID NO:2, and a CDR3 comprising an amino acid sequence of SEQ ID NO: 3; and a light chain polypeptide comprising CDR1 comprising an amino acid sequence of SEQ ID NO:4, a CDR2 comprising an amino acid sequence of SEQ ID NO:5, and a CDR3 comprising an amino acid sequence of SEQ ID NO:6. In some embodiments, the antibody comprises a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9; and a light chain polypeptide comprising CDR1 comprising an amino acid sequence of SEQ ID NO:10, a CDR2 comprising an amino acid sequence of SEQ ID NO:11, and a CDR3 comprising an amino acid sequence of SEQ ID NO:12. In some embodiments, the antibody comprises a heavy chain polypeptide comprising a CDR1 comprising an amino acid sequence of SEQ ID NO: 13, a CDR2 comprising an amino acid sequence of SEQ ID NO:14, and a CDR3 comprising an amino acid sequence of SEQ ID NO:15; and a light chain polypeptide comprising CDR1 comprising an amino acid sequence of SEQ ID NO:4, a CDR2 comprising an amino acid sequence of SEQ ID NO:16, and a CDR3 comprising an amino acid sequence of SEQ ID NO: 17. In some embodiments, the antibody comprises a heavy chain polypeptide comprising a CDR1 comprising an amino acid sequence of SEQ ID NO: 18, a CDR2 comprising an amino acid sequence of SEQ ID NO:19, and a CDR3 comprising an amino acid sequence of SEQ ID NO:20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22. In some embodiments, the antibody comprises a heavy chain polypeptide comprising a CDR1 comprising an amino acid sequence of SEQ ID NO:23, a CDR2 comprising an amino acid sequence of SEQ ID NO:24, and a CDR3 comprising an amino acid sequence of SEQ ID NO: 25; and a light chain polypeptide comprising CDR1 comprising an amino acid sequence of SEQ ID NO:26, a CDR2 comprising an amino acid sequence of SEQ ID NO:27, and a CDR3 comprising an amino acid sequence of SEQ ID NO:28.

In some embodiments, the heavy chain polypeptide of the antibody comprises a heavy chain variable region comprising an amino acid sequence of SEQ ID NO:29. In some embodiments, the heavy chain polypeptide of the antibody comprises a heavy chain variable region comprising an amino acid sequence of SEQ ID NO:31. In some embodiments, the heavy chain polypeptide of the antibody comprises a heavy chain variable region comprising an amino acid sequence of SEQ ID NO:33. In some embodiments, the heavy chain polypeptide of the antibody comprises a heavy chain variable region comprising an amino acid sequence of SEQ ID NO:35. In some embodiments, the heavy chain polypeptide of the antibody comprises a heavy chain variable region comprising an amino acid sequence of SEQ ID NO: 37. In some embodiments, the heavy chain polypeptide of the antibody comprises a heavy chain variable region comprising an amino acid sequence of SEQ ID NO:87;

In some embodiments, the light chain polypeptide of the antibody comprises a light chain variable region comprising an amino acid sequence of SEQ ID NO:30. In some embodiments, the light chain polypeptide of the antibody comprises a light chain variable region comprising an amino acid sequence of SEQ ID NO:32. In some embodiments, the light chain polypeptide of the antibody comprises a light chain variable region comprising an amino acid sequence of SEQ ID NO:34. In some embodiments, the light chain polypeptide of the antibody comprises a light chain variable region comprising an amino acid sequence of SEQ ID NO:36. In some embodiments, the light chain polypeptide of the antibody comprises a light chain variable region comprising an amino acid sequence of SEQ ID NO:38. In some embodiments, the light chain polypeptide of the antibody comprises a light chain variable region comprising an amino acid sequence of SEQ ID NO:88.

In some embodiments, the antibody comprises a heavy chain polypeptide comprising a heavy chain variable region comprising an amino acid sequence of SEQ ID NO:29; and a light chain peptide comprising a light chain variable region comprising an amino acid sequence of SEQ ID NO:30. In some embodiments, the antibody comprises a heavy chain polypeptide comprising a heavy chain variable region comprising an amino acid sequence of SEQ ID NO:31; and a light chain peptide comprising a light chain variable region comprising an amino acid sequence of SEQ ID NO:32. In some embodiments, the antibody comprises a heavy chain polypeptide comprising a heavy chain variable region comprising an amino acid sequence of SEQ ID NO:33; and a light chain peptide comprising a light chain variable region comprising an amino acid sequence of SEQ ID NO:34. In some embodiments, the antibody comprises a heavy chain polypeptide comprising a heavy chain variable region comprising an amino acid sequence of SEQ ID NO:35; and a light chain peptide comprising a light chain variable region comprising an amino acid sequence of SEQ ID NO:36. In some embodiments, the antibody comprises a heavy chain polypeptide comprising a heavy chain variable region comprising an amino acid sequence of SEQ ID NO:37; a light chain peptide comprising a light chain variable region comprising an amino acid sequence of SEQ ID NO: 38. In some embodiments, the antibody comprises a heavy chain polypeptide comprising a heavy chain variable region comprising an amino acid sequence of SEQ ID NO:87; a light chain peptide comprising a light chain variable region comprising an amino acid sequence of SEQ ID NO:88.

In some embodiments, the antibody binds to the extracellular portion of tumor necrosis factor receptor 2 (TNFR2).

In some embodiments, the antibody binds to soluble tumor necrosis factor receptor 2 (STNFR2).

In some embodiments, the TNFR2 and/or the sTNFR2 are human TNFR2 and/or human sTNFR2.

In some embodiments, the antibody does not bind to soluble tumor necrosis factor receptor 1 (sTNFR1).

In some embodiments, the antibody is isolated.

In some embodiments, the antibody is recombinant.

In some embodiments, the antibody is an IgG, IgM, IgA, or an antigen binding fragment thereof.

In some embodiments, the antibody the antibody is a Fab′, a F(ab′)2, a F(ab′)3, a monovalent scFv, a bivalent scFv, a single domain antibody, or a minibody.

In some embodiments, the antibody the antibody is a full length antibody.

In some embodiments, the antibody the antibody is a human antibody, a humanized antibody, or a de-immunized antibody.

In some aspects, the present invention is directed to a nucleic acid encoding an antibody.

In some embodiments, the nucleic acid comprises a first nucleic acid and/or a second nucleic acid.

In some embodiments, the first nucleic acid comprises a first portion encoding an amino acid sequence of SEQ ID NO: 1; a second portion encoding an amino acid sequence of SEQ ID NO:2; and a third portion encoding an amino acid sequence of SEQ ID NO:3. In some embodiments, the first nucleic acid comprises a first portion encoding an amino acid sequence of SEQ ID NO:7; a second portion encoding an amino acid sequence of SEQ ID NO: 8; and a third portion encoding an amino acid sequence of SEQ ID NO:9. In some embodiments, the first nucleic acid comprises a first portion encoding an amino acid sequence of SEQ ID NO: 13; a second portion encoding an amino acid sequence of SEQ ID NO: 14; and a third portion encoding an amino acid sequence of SEQ ID NO:15. In some embodiments, the first nucleic acid comprises a first portion encoding an amino acid sequence of SEQ ID NO: 18; a second portion encoding an amino acid sequence of SEQ ID NO: 19; and a third portion encoding an amino acid sequence of SEQ ID NO:20. In some embodiments, the first nucleic acid comprises a first portion encoding an amino acid sequence of SEQ ID NO:23; a second portion encoding an amino acid sequence of SEQ ID NO: 24; and a third portion encoding an amino acid sequence of SEQ ID NO:25.

In some embodiments, the second nucleic acid comprises a first portion encoding an amino acid sequence of SEQ ID NO:4; a second portion encoding an amino acid sequence of SEQ ID NO:5; and a third portion encoding an amino acid sequence of SEQ ID NO:6. In some embodiments, the second nucleic acid comprises a first portion encoding an amino acid sequence of SEQ ID NO: 10; a second portion encoding an amino acid sequence of SEQ ID NO: 11; and a third portion encoding an amino acid sequence of SEQ ID NO:12. In some embodiments, the second nucleic acid comprises a first portion encoding an amino acid sequence of SEQ ID NO:4; a second portion encoding an amino acid sequence of SEQ ID NO: 16; and a third portion encoding an amino acid sequence of SEQ ID NO:17. In some embodiments, the second nucleic acid comprises a first portion encoding an amino acid sequence of SEQ ID NO:4; a second portion encoding an amino acid sequence of SEQ ID NO: 21; and a third portion encoding an amino acid sequence of SEQ ID NO:22. In some embodiments, the second nucleic acid comprises a first portion encoding an amino acid sequence of SEQ ID NO:26; a second portion encoding an amino acid sequence of SEQ ID NO: 27; and a third portion encoding an amino acid sequence of SEQ ID NO:28.

In some embodiments, the first nucleic acid comprises a first portion comprising the sequence of SEQ ID NO:39; a second portion comprising the sequence of SEQ ID NO:40; and a third portion comprising the sequence of SEQ ID NO:41. In some embodiments, the first nucleic acid comprises a first portion comprising the sequence of SEQ ID NO:45; a second portion comprising the sequence of SEQ ID NO:46; and a third portion comprising a sequence of SEQ ID NO:47. In some embodiments, the first nucleic acid comprises a first portion comprising the sequence of SEQ ID NO:51; a second portion comprising the sequence of SEQ ID NO:52; and a third portion comprising the sequence of SEQ ID NO:53. In some embodiments, the first nucleic acid comprises a first portion comprising the sequence of SEQ ID NO:56; a second portion comprising the sequence of SEQ ID NO:57; and a third portion comprising a sequence of SEQ ID NO:58. In some embodiments, the first nucleic acid comprises a first portion comprising the sequence of SEQ ID NO:62; a second portion comprising the sequence of SEQ ID NO:63; and a third portion comprising the sequence of SEQ ID NO:64.

In some embodiments, the second nucleic acid comprises a first portion comprising the sequence of SEQ ID NO:42; a second portion comprising the sequence of SEQ ID NO: 43; and a third portion comprising the sequence of SEQ ID NO:44. In some embodiments, the second nucleic acid comprises a first portion comprising the sequence of SEQ ID NO:48; a second portion comprising the sequence of SEQ ID NO:49; and a third portion comprising a sequence of SEQ ID NO:50. In some embodiments, the second nucleic acid comprises a first portion comprising the sequence of SEQ ID NO:42; a second portion comprising the sequence of SEQ ID NO:54; and a third portion comprising the sequence of SEQ ID NO:55. In some embodiments, the second nucleic acid comprises a first portion comprising the sequence of SEQ ID NO:59; a second portion comprising the sequence of SEQ ID NO:60; and a third portion comprising a sequence of SEQ ID NO:61. In some embodiments, the second nucleic acid comprises a first portion comprising the sequence of SEQ ID NO:65; a second portion comprising the sequence of SEQ ID NO:66; and a third portion comprising the sequence of SEQ ID NO:67.

In some embodiments, the first nucleic acid comprises a nucleic acid encoding a heavy chain variable region comprising an amino acid sequence of SEQ ID NO:29. In some embodiments, the second nucleic acid comprises a nucleic acid encoding a heavy chain variable region comprising an amino acid sequence of SEQ ID NO:31. In some embodiments, the second nucleic acid comprises a nucleic acid encoding a heavy chain variable region comprising an amino acid sequence of SEQ ID NO:33. In some embodiments, the second nucleic acid comprises a nucleic acid encoding a heavy chain variable region comprising an amino acid sequence of SEQ ID NO:35. In some embodiments, the second nucleic acid comprises a nucleic acid encoding a heavy chain variable region comprising an amino acid sequence of SEQ ID NO:37. In some embodiments, the second nucleic acid comprises a nucleic acid encoding a heavy chain variable region comprising an amino acid sequence of SEQ ID NO:87.

In some embodiments, the second nucleic acid comprises a nucleic acid encoding a light chain variable region comprising an amino acid sequence of SEQ ID NO:30. In some embodiments, the second nucleic acid comprises a nucleic acid encoding a light chain variable region comprising an amino acid sequence of SEQ ID NO:32. In some embodiments, the second nucleic acid comprises a nucleic acid encoding a light chain variable region comprising an amino acid sequence of SEQ ID NO:34. In some embodiments, the second nucleic acid comprises a nucleic acid encoding a light chain variable region comprising an amino acid sequence of SEQ ID NO:36. In some embodiments, the second nucleic acid comprises a nucleic acid encoding a light chain variable region comprising an amino acid sequence of SEQ ID NO:38. In some embodiments, the second nucleic acid comprises a nucleic acid encoding a light chain variable region comprising an amino acid sequence of SEQ ID NO:88.

In some embodiments, the nucleic acid comprises a first nucleic acid comprising a nucleic acid encoding a heavy chain variable region comprising an amino acid sequence of SEQ ID NO:29; and a second nucleic acid comprising a nucleic acid encoding a light chain variable region comprising an amino acid sequence of SEQ ID NO:30. In some embodiments, the nucleic acid comprises a first nucleic acid comprising a nucleic acid encoding a heavy chain variable region comprising an amino acid sequence of SEQ ID NO: 31; and a second nucleic acid comprising a nucleic acid encoding a light chain variable region comprising an amino acid sequence of SEQ ID NO:32. In some embodiments, the nucleic acid comprises a first nucleic acid comprising a nucleic acid encoding a heavy chain variable region comprising an amino acid sequence of SEQ ID NO:33; and a second nucleic acid comprising a nucleic acid encoding a light chain variable region comprising an amino acid sequence of SEQ ID NO:34. In some embodiments, the nucleic acid comprises a first nucleic acid comprising a nucleic acid encoding a heavy chain variable region comprising an amino acid sequence of SEQ ID NO:35; and a second nucleic acid comprising a nucleic acid encoding a light chain variable region comprising an amino acid sequence of SEQ ID NO:36. In some embodiments, the nucleic acid comprises a first nucleic acid comprising a nucleic acid encoding a heavy chain variable region comprising an amino acid sequence of SEQ ID NO: 37; and a second nucleic acid comprising a nucleic acid encoding a light chain variable region comprising an amino acid sequence of SEQ ID NO:38. In some embodiments, the nucleic acid comprises a first nucleic acid comprising a nucleic acid encoding a heavy chain variable region comprising an amino acid sequence of SEQ ID NO:87; and a second nucleic acid comprising a nucleic acid encoding a light chain variable region comprising an amino acid sequence of SEQ ID NO:88.

In some embodiments, the first nucleic acid comprises a nucleic acid comprising the sequence of SEQ ID NO:68. In some embodiments, the first nucleic acid comprises a nucleic acid comprising the sequence of SEQ ID NO:70. In some embodiments, the first nucleic acid comprises a nucleic acid comprising the sequence of SEQ ID NO:72. In some embodiments, the first nucleic acid comprises a nucleic acid comprising the sequence of SEQ ID NO:74. In some embodiments, the first nucleic acid comprises a nucleic acid comprising the sequence of SEQ ID NO:76. In some embodiments, the first nucleic acid comprises a nucleic acid comprising the sequence of SEQ ID NO:89.

In some embodiments, the second nucleic acid comprises a nucleic acid comprising the sequence of SEQ ID NO:69. In some embodiments, the second nucleic acid comprises a nucleic acid comprising the sequence of SEQ ID NO:71. In some embodiments, the second nucleic acid comprises a nucleic acid comprising the sequence of SEQ ID NO:73. In some embodiments, the second nucleic acid comprises a nucleic acid comprising the sequence of SEQ ID NO:75. In some embodiments, the second nucleic acid comprises a nucleic acid comprising the sequence of SEQ ID NO:77. In some embodiments, the second nucleic acid comprises a nucleic acid comprising the sequence of SEQ ID NO:90.

In some embodiments, the nucleic acid comprises a first nucleic acid comprising the sequence of SEQ ID NO:68; and a second nucleic acid comprising the sequence of SEQ ID NO: 69. In some embodiments, the nucleic acid comprises a first nucleic acid comprising the sequence of SEQ ID NO: 70; and a second nucleic acid comprising the sequence of SEQ ID NO: 71. In some embodiments, the nucleic acid comprises a first nucleic acid comprising the sequence of SEQ ID NO:72; and a second nucleic acid comprising the sequence of SEQ ID NO: 73. In some embodiments, the nucleic acid comprises a first nucleic acid comprising the sequence of SEQ ID NO:74; and a second nucleic acid comprising the sequence of SEQ ID NO: 75. In some embodiments, the nucleic acid comprises a first nucleic acid comprising the sequence of SEQ ID NO:76; and a second nucleic acid comprising the sequence of SEQ ID NO: 77. In some embodiments, the nucleic acid comprises a first nucleic acid comprising the sequence of SEQ ID NO:89; and a second nucleic acid comprising the sequence of SEQ ID NO: 90.

In some embodiments, the nucleic acid comprises an expression vector.

In some aspects, the present invention is directed to an antibody pair comprising a first antibody and a second antibody.

In some embodiments, the first antibody comprises a heavy chain polypeptide comprising a CDR1 comprising an amino acid sequence of SEQ ID NO: 1, a CDR2 comprising an amino acid sequence of SEQ ID NO:2, and a CDR3 comprising an amino acid sequence of SEQ ID NO:3; and a light chain polypeptide comprising CDR1 comprising an amino acid sequence of SEQ ID NO:4, a CDR2 comprising an amino acid sequence of SEQ ID NO: 5, and a CDR3 comprising an amino acid sequence of SEQ ID NO:6.

In some embodiments, the second antibody comprises a heavy chain polypeptide comprising a CDR1 comprising an amino acid sequence of SEQ ID NO:1, a CDR2 comprising an amino acid sequence of SEQ ID NO:2, and a CDR3 comprising an amino acid sequence of SEQ ID NO:3; and a light chain polypeptide comprising CDR1 comprising an amino acid sequence of SEQ ID NO:4, a CDR2 comprising an amino acid sequence of SEQ ID NO: 5, and a CDR3 comprising an amino acid sequence of SEQ ID NO:6.

In some embodiments, the first antibody comprises a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9; and a light chain polypeptide comprising CDR1 comprising an amino acid sequence of SEQ ID NO: 10, a CDR2 comprising an amino acid sequence of SEQ ID NO: 11, and a CDR3 comprising an amino acid sequence of SEQ ID NO: 12.

In some embodiments, the second antibody comprises a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9; and a light chain polypeptide comprising CDR1 comprising an amino acid sequence of SEQ ID NO: 10, a CDR2 comprising an amino acid sequence of SEQ ID NO: 11, and a CDR3 comprising an amino acid sequence of SEQ ID NO: 12.

In some embodiments, the first antibody comprises a heavy chain polypeptide comprising a CDR1 comprising an amino acid sequence of SEQ ID NO: 13, a CDR2 comprising an amino acid sequence of SEQ ID NO:14, and a CDR3 comprising an amino acid sequence of SEQ ID NO:15; and a light chain polypeptide comprising CDR1 comprising an amino acid sequence of SEQ ID NO:4, a CDR2 comprising an amino acid sequence of SEQ ID NO:16, and a CDR3 comprising an amino acid sequence of SEQ ID NO:17.

In some embodiments, the second antibody comprises a heavy chain polypeptide comprising a CDR1 comprising an amino acid sequence of SEQ ID NO: 13, a CDR2 comprising an amino acid sequence of SEQ ID NO:14, and a CDR3 comprising an amino acid sequence of SEQ ID NO: 15; and a light chain polypeptide comprising CDR1 comprising an amino acid sequence of SEQ ID NO:4, a CDR2 comprising an amino acid sequence of SEQ ID NO:16, and a CDR3 comprising an amino acid sequence of SEQ ID NO:17.

In some embodiments, the first antibody comprises a heavy chain polypeptide comprising a CDR1 comprising an amino acid sequence of SEQ ID NO: 18, a CDR2 comprising an amino acid sequence of SEQ ID NO:19, and a CDR3 comprising an amino acid sequence of SEQ ID NO:20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22.

In some embodiments, the second antibody comprises a heavy chain polypeptide comprising a CDR1 comprising an amino acid sequence of SEQ ID NO: 18, a CDR2 comprising an amino acid sequence of SEQ ID NO:19, and a CDR3 comprising an amino acid sequence of SEQ ID NO:20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22.

In some embodiments, the first antibody comprises a heavy chain polypeptide comprising a CDR1 comprising an amino acid sequence of SEQ ID NO:23, a CDR2 comprising an amino acid sequence of SEQ ID NO:24, and a CDR3 comprising an amino acid sequence of SEQ ID NO:25; and a light chain polypeptide comprising CDR1 comprising an amino acid sequence of SEQ ID NO:26, a CDR2 comprising an amino acid sequence of SEQ ID NO:27, and a CDR3 comprising an amino acid sequence of SEQ ID NO:28.

In some embodiments, the second antibody comprises a heavy chain polypeptide comprising a CDR1 comprising an amino acid sequence of SEQ ID NO:23, a CDR2 comprising an amino acid sequence of SEQ ID NO:24, and a CDR3 comprising an amino acid sequence of SEQ ID NO:25; and a light chain polypeptide comprising CDR1 comprising an amino acid sequence of SEQ ID NO:26, a CDR2 comprising an amino acid sequence of SEQ ID NO:27, and a CDR3 comprising an amino acid sequence of SEQ ID NO:28.

In some embodiments, the first antibody and the second antibody does not compete for the same epitope on sTNFR2.

In some embodiments, the first antibody comprises a heavy chain polypeptide comprising a CDR1 comprising an amino acid sequence of SEQ ID NO: 18, a CDR2 comprising an amino acid sequence of SEQ ID NO:19, and a CDR3 comprising an amino acid sequence of SEQ ID NO:20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22.

In some embodiments, the second antibody comprises a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9; and a light chain polypeptide comprising CDR1 comprising an amino acid sequence of SEQ ID NO: 10, a CDR2 comprising an amino acid sequence of SEQ ID NO: 11, and a CDR3 comprising an amino acid sequence of SEQ ID NO: 12.

In some embodiments, the first antibody comprises a heavy chain polypeptide comprising a heavy chain variable region comprising an amino acid sequence of SEQ ID NO: 29; and a light chain peptide comprising a light chain variable region comprising an amino acid sequence of SEQ ID NO:30.

In some embodiments, the second antibody comprises a heavy chain polypeptide comprising a heavy chain variable region comprising an amino acid sequence of SEQ ID NO: 29; and a light chain peptide comprising a light chain variable region comprising an amino acid sequence of SEQ ID NO:30.

In some embodiments, the first antibody comprises a heavy chain polypeptide comprising a heavy chain variable region comprising an amino acid sequence of SEQ ID NO: 31; and a light chain peptide comprising a light chain variable region comprising an amino acid sequence of SEQ ID NO:32.

In some embodiments, the second antibody comprises a heavy chain polypeptide comprising a heavy chain variable region comprising an amino acid sequence of SEQ ID NO: 31; and a light chain peptide comprising a light chain variable region comprising an amino acid sequence of SEQ ID NO:32.

In some embodiments, the first antibody comprises a heavy chain polypeptide comprising a heavy chain variable region comprising an amino acid sequence of SEQ ID NO: 33; and a light chain peptide comprising a light chain variable region comprising an amino acid sequence of SEQ ID NO:34.

In some embodiments, the second antibody comprises a heavy chain polypeptide comprising a heavy chain variable region comprising an amino acid sequence of SEQ ID NO: 33; and a light chain peptide comprising a light chain variable region comprising an amino acid sequence of SEQ ID NO:34.

In some embodiments, the first antibody comprises a heavy chain polypeptide comprising a heavy chain variable region comprising an amino acid sequence of SEQ ID NO: 35; and a light chain peptide comprising a light chain variable region comprising an amino acid sequence of SEQ ID NO:36.

In some embodiments, the second antibody comprises a heavy chain polypeptide comprising a heavy chain variable region comprising an amino acid sequence of SEQ ID NO: 35; and a light chain peptide comprising a light chain variable region comprising an amino acid sequence of SEQ ID NO:36.

In some embodiments, the first antibody comprises a heavy chain polypeptide comprising a heavy chain variable region comprising an amino acid sequence of SEQ ID NO: 37; and a light chain peptide comprising a light chain variable region comprising an amino acid sequence of SEQ ID NO:38.

In some embodiments, the second antibody comprises a heavy chain polypeptide comprising a heavy chain variable region comprising an amino acid sequence of SEQ ID NO: 37; and a light chain peptide comprising a light chain variable region comprising an amino acid sequence of SEQ ID NO:38.

In some embodiments, the first antibody comprises a heavy chain polypeptide comprising a heavy chain variable region comprising an amino acid sequence of SEQ ID NO: 87; and a light chain peptide comprising a light chain variable region comprising an amino acid sequence of SEQ ID NO:88.

In some embodiments, the first antibody comprises a heavy chain polypeptide comprising a heavy chain variable region comprising an amino acid sequence of SEQ ID NO: 35; and a light chain peptide comprising a light chain variable region comprising an amino acid sequence of SEQ ID NO:36, or the first antibody comprises a heavy chain polypeptide comprising a heavy chain variable region comprising an amino acid sequence of SEQ ID NO:87; and a light chain peptide comprising a light chain variable region comprising an amino acid sequence of SEQ ID NO:88. In some embodiments, the second antibody comprises a heavy chain polypeptide comprising a heavy chain variable region comprising an amino acid sequence of SEQ ID NO:31; and a light chain peptide comprising a light chain variable region comprising an amino acid sequence of SEQ ID NO:32.

In some embodiments, the antibody pair quantifies sTNFR2 in a sample. In some embodiments, the first antibody captures sTNFR2 in the sample and the second antibody quantifies the sTNFR2 captured by the first antibody.

In some aspects, the present invention is directed to a method of quantifying sTNFR2 in a sample.

In some embodiments, the method uses an antibody pair, such as the antibody pair described herein, such as the antibody pair described above.

In some embodiments, the method comprises capturing sTNFR2 in the sample with the first antibody; and quantifying sTNFR2 captured by the first antibody with the second antibody.

In some embodiments, the method of quantifying is a western blotting-based method, an enzyme-linked immunoassay (ELISA)-based method, or combinations thereof.

In some embodiments, the sample is a serum sample, a plasma sample, a urine sample, or combinations thereof.

In some embodiments, the sample is a sample from a subject suspected to have a disease or disorder indicated by altered levels of sTNFR2, and the quantification of sTNFR2 in the sample diagnosis the disease or disorder.

In some embodiments, the disease is Kawasaki disease (K_{D}) or Kawasaki-like syndrome (KLS).

In some embodiments, the sample is a sample from a subject confirmed to have a disease or disorder involving altered levels of sTNFR2. In some embodiments, the quantification of sTNFR2 predicts outcomes of the disease or disorder or monitors the state of the disease or disorder during therapies.

In some embodiments, the disease or disorder is lupus nephritis, Behçet's disease, Sjogren's syndrome, rheumatoid arthritis and juvenile chronic arthritis, inflammatory bowel diseases, sarcoidosis, Grave's disease, myocardial infarction, congestive heart failure, progressive diabetic kidney disease in subjects with type 2 diabetes mellitus, erythropoietin resistance in setting of hemodialysis, steatohepatitis, hepatitis C virus liver fibrosis, post-COVID-19 multisystem inflammatory syndrome (MIS-C) in children, presence and/or prognosis of infections including sepsis, malaria, COVID-19, or HIV.

In some aspects, the present invention is directed to a method of diagnosing in a subject a disease or disorder involving altered levels of sTNFR2.

In some embodiments, the method comprises: capturing sTNFR2 in a sample from the subject with the first antibody; and quantifying sTNFR2 captured by the first antibody with the second antibody. In some embodiments, the first antibody and/or the second antibody is an antibody described herein, such as an antibody described above in this section.

In some embodiments, if the sTNFR2 level quantified by the first antibody and the second antibody is equal to or higher than a first predetermined level, the subject is diagnosed as having the disease or disorder.

In some embodiments, if the sTNFR2 level quantified by the first antibody and the second antibody is lower than a first predetermined level, the subject is diagnosed as not having the disease or disorder.

In some embodiments, the method of quantifying is a western blotting-based method, an enzyme-linked immunoassay (ELISA)-based method, or combinations thereof.

In some embodiments, the sample is a serum sample, a plasma sample, a urine sample, or combinations thereof.

In some embodiments, the disease is Kawasaki disease (K_{D}) or Kawasaki-like syndrome (KLS).

In some aspects, the present invention is directed to a method of monitoring progression and/or state of a disease or disorder involving altered levels of sTNFR2 in a subject diagnosed with the disease or disorder.

In some embodiments, the method comprises: obtaining subject's samples at two or more time points after diagnosis of the disease or disorder in the subject; capturing sTNFR2 in each obtained sample with a first antibody; quantifying sTNFR2 captured by the first antibody with a second antibody; and comparing the sTNFR2 levels quantified by the first antibody and the second antibody in the obtained subject's samples. In some embodiments, the first antibody and/or the second antibody are an antibody described herein, such as an antibody described in this section.

In some embodiments, the comparing step allows for monitoring progression and/or state of the disease or disorder in the subject.

In some embodiments, at least one of the two or more time points is a time point where the subject is receiving therapeutic treatment for the disease or disorder. In some embodiments, the comparing step allows for monitoring therapeutic outcome of the therapeutic treatment in the subject.

In some embodiments, the method of quantifying is a western blotting-based method, an enzyme-linked immunoassay (ELISA)-based method, or combinations thereof.

In some embodiments, each sample is independently a serum sample, a plasma sample, a urine sample, or combinations thereof.

In some embodiments, the disease or disorder is lupus nephritis, Behçet's disease, Sjogren's syndrome, rheumatoid arthritis and juvenile chronic arthritis, inflammatory bowel diseases, sarcoidosis, Grave's disease, myocardial infarction, congestive heart failure, progressive diabetic kidney disease in subjects with type 2 diabetes mellitus, erythropoietin resistance in setting of hemodialysis, steatohepatitis, hepatitis C virus liver fibrosis, post-COVID-19 multisystem inflammatory syndrome (MIS-C) in children, presence and/or prognosis of infections including sepsis, malaria, COVID-19, or HIV.

In some aspects, the present invention is directed to a method of treating, ameliorating, and/or preventing Kawasaki disease or Kawasaki-like syndrome in a subject in need thereof.

In some embodiments, the method comprises: quantifying sTNFR2 level in a sample from the subject, comprising capturing sTNFR in the sample with a first antibody, and quantifying the sTNFR2 captured by the first antibody with a second antibody; and administering to the subject an effective amount of a medication for treating, ameliorating, and/or preventing Kawasaki disease or Kawasaki-like syndrome if the sTNFR2 level quantified by the first antibody and the second antibody is equal to or higher than a first predetermined level. In some embodiments, the first antibody and/or the second antibody are an antibody described herein, such as in this section.

In some embodiments, the sample is a serum sample, a plasma sample, a urine sample, or combinations thereof.

In some embodiments, the method further comprises: quantifying C—C motif chemokine ligand 1 (Ccl1) level in the sample; quantifying C—C motif chemokine ligand 2 (Ccl2) level in the sample; and quantifying C—X—C motif chemokine 11 (Cxc11) level in the sample.

In some embodiments, the subject is administered with the medication for treating, ameliorating, and/or preventing Kawasaki disease or Kawasaki-like syndrome if the sTNFR2 level quantified by the first antibody and the second antibody is equal to or higher than a first predetermined level, and if at least two of the following are true:
(a) the quantified Ccl1 level is higher than a second predetermined level,
(b) the quantified Ccl2 level is higher than a third predetermined level,
(c) the quantified Cxc11 level is higher than a fourth predetermined level.

In some embodiments, the medication comprises an intravenous immunoglobulin (IVIG), aspirin, a steroid, or combinations thereof.

In some aspects, the present invention is directed to a method of treating, ameliorating and/or preventing an inflammatory disease or disorder in a subject in need thereof.

In some embodiments, the method comprises: administering to the subject an effective amount of an antibody. In some embodiments, the inflammatory disease is caused by or involves overactive TNFα-TNFR2 signaling. In some embodiments, the antibody is an antibody described herein, such as an antibody described in this section.

In some embodiments, the disease or disorder is at least one selected from the group consisting of lupus nephritis, plaque psoriasis, psoriatic arthritis, ankylosing spondylitis, Crohn's disease, ulcerative colitis, hidradenitis, uveitis, Kawasaki disease, Behçet's disease, Sjogren's syndrome, rheumatoid arthritis and juvenile chronic arthritis, juvenile rheumatoid arthritis with macrophage activation syndrome, inflammatory bowel diseases, sarcoidosis, Grave's disease, myocardial infarction, congestive heart failure, progressive diabetic kidney disease in subjects with type 2 diabetes mellitus, steatohepatitis, hepatitis C virus liver fibrosis, post-COVID-19 multisystem inflammatory syndrome (MIS-C) in children and adults (MIS-A), bacterial sepsis, interstitial lung disease, cerebral malaria, acute respiratory distress syndrome (ARDS) or diffuse alveolar hemorrhage (DAH) associated with viral infections comprising COVID-19, and bacterial infections comprising leptospirosis.

In some embodiments, the method reduces a level of interferon γ (IFN-γ) in the subject.

In some embodiments, the method reduces an expression of IFN-γ by the peripheral blood mononuclear cells (PBMCs).

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1B illustrates certain aspects of the fusion proteins used to generate the antibodies, in accordance with some embodiments. FIG. 1A: Plasmid map of pFuse-ratIgG2b-Fc2 used to generate the plasmids pFuse-ratIgG2b-Fc2-sTNFR2sig1 and pFuse-ratIgG2b-Fc2-sTNFR2sig2, which were used to express sTNFR2-rat IgG Fc fusion proteins STNFR2sig1 and sTNFR2sig2. FIG. 1B: Gel electrophoresis and staining results of purified STNFR2sig1 and sTNFR2sig2 fusion proteins.

FIGS. 2A-2B illustrate certain aspects of the detection of human sTNFR2 by pairs of sTNFR2 monoclonal antibodies drawn from hsTNFR2-26C9, -1G2, -6D7, -9C10, -30C5, in accordance with some embodiments. Indicated capture antibody at 2 ug/ml in PBS, 50 ul per well, was used to coated Nunc maxisorp plates (Invitrogen cat #44-2404-21) overnight at 4 °C. Biotinylated detection antibodies were used at 250 ng/ml, 100 μl per well (biotinylation reagent ThermoFisher Pierce cat #90407). Concentrations of sTNFR2 in healthy control (HC) plasma (4300 pg/ml; 1:30=143 pg/ml; 1:300=14.3 pg/ml), severe COVID-19 (COV) plasma (28,900 pg/ml; 1:30=963 pg/ml; 1:300=96.3 pg/ml), and sTNFR2sig1 at 2000 pg/ml. sTNFR2sig1 produced in 293F cells may have denatured or unique epitopes based on construct and purification methodology. COV 1:30 to sTNFR2 ratios <2.1 (2000/963=2.1) Several antibody pairs captured and detected epitopes intact in native sTNFR2 and STNFR2sig1 protein (26C9, 1G2, 6D7, 9C10). Monoclonal antibody hsTNFR2-30C5 recognizes a sTNFR2sig1 epitope that is more accessible in sTNFR2sig1 than native sTNFR2 (ratio >2.1). 1G2 and 9C10 are competitive with each other (have overlapping epitopes that don't work as a capture-detection antibody pair). Zero=1.5% BSA/0.05% Tween20/PBS blocked well without plasma or recombinant sTNFR2 added, with each antibody capture-detection pair (control well). Colorometric development reagents=Streptavidin-HRP (BD Pharmingen Cat #554066; used at 1:6000 in blocking buffer; 100 ul) and TMB substrate (Sigma cat #T-4444; 100 μl). Stop reagent=0.32 M H₂SO₄ (50 μl).

FIG. 3 depicts the comparison among sTNFR2 quantifications results obtained using the Aushon Biosystems using a multiplex ELISA (undisclosed proprietary antibodies) (data published in Johnson et al., Open Forum Infect Dis 2016; 3:ofw160) (“Pub”), the R & D systems sTNFR2 duoset (polyclonal detection antibody) (“R & D”), and the instant 26C09/6D7 sTNFR2 ELISA (monoclonal antibody pair) (“RMJ”), in accordance with some embodiments. The sTNFR2 levels were determined in serum samples from healthy subjects, febrile controls who do not have Kawasaki disease, and Kawasaki patients. The results demonstrate that the non-limiting examples of the instant antibodies (RMJ) have higher sensitivity as they were able to detect more sTNFR2 in all three groups of samples.

FIG. 4 depicts the comparison between sTNFR2 quantifications results obtained using the R & D systems sTNFR2 duoset (polyclonal detection antibody) (“R & D”), and the instant 26C09/6D7 sTNFR2 ELISA (monoclonal antibody pair) (“RMJ”), in accordance with some embodiments. The levels of sTNFR2 were determined in sera of a healthy control subjects and a patient with severe COVID-19. The results demonstrate that the non-limiting examples of the instant antibodies have higher sensitivity as they were able to detect more sTNFR2 in both serum samples with p value <0.05 (*).

FIG. 5 depicts that the non-limiting examples of the instant antibodies have no cross-reactivity with sTNFR1, in accordance with some embodiments, sTNFR1 levels in the COVID patient plasma were measured with R & D systems Human TNF RI/TNFRSF1A DuoSet ELISA cat #DY225 (“R & D systems”) separately substituting 26C09 for the R & D systems capture antibody (“26C09”) and 6D7biotinylated for the detection polyclonal antibody (“6D7”). Neither of the non-limiting examples of the antibodies herein have detectable cross-reactivity with sTNFR1. The plasma of the patient with severe COVID-19 had a sTNFR1 level of 1700+/−300 pg/ml as determined by the R & D Systems Human TNF RI/TNFRSF1A DuoSet ELISA.

FIG. 6 is a diagram of an algorithm for diagnosing Kawasaki Disease (K_{D}) and Kawasaki-like Syndrome (KLS) from a published investigation (Johnson et al., Open Forum Infect Dis 2016; 3: ofw160).

FIGS. 7A-7B show a schematic for the static TNFα binding assay and the dynamic TNFα biologic activity assay, in accordance with some embodiments. FIG. 7A: Static assay. Immobilized sTNFR2sig1, untreated and exposed to monoclonal antibodies specific for TNFR2, is then exposed to recombinant human TNFα. Monoclonal antibodies such as STNFR2-26C9 that obstruct the binding site or detrimentally alter the conformation of TNFR2 block the binding of TNFα to sTNFR2. Binding of TNFα indicates that this solid phase assay recapitulates trimerization of TNFR2 necessary to bind TNFα. FIG. 7B: Dynamic assay. PBMC are untreated or transiently exposed to monoclonal antibodies specific for CD3 & CD28, without or with monoclonal antibody directed at an investigatory costimulatory receptor (e.g. TNFR2; 26C9), then washed an plated in replicate wells of a 96 well plate containing IL-2 and IL-7, without or with additional inhibitory agents (e.g. Fab fragments of 26C9). Antibodies that are washed away deliver transient signals. Inhibitors in the wells are present continuously throughout an experiment. Specifically, washing away antibodies resulted in a transient activation (CD3 & CD28) and co-stimulation signal (TNFR2). Inhibitors of the TNFR2 signal were presented transiently (in the case of whole 26C9 antibody) or continuously (in the case of fragmented 26C9 antibody).

FIGS. 8A-8B show binding of recombinant human TNFα to the TNFR2 fusion protein designated sTNFR2sig1 (static assay), and screening of a panel of anti-TNFR2 antibodies for their ability to block TNFα binding using the static assay, thereby identifying TNFR2-26C9 as a blocking antibody, in accordance with some embodiments. FIG. 8A: sTNFR2sig1 coated wells (100 ng/well) are pre-exposed to indicated monoclonal antibodies prior to addition of 50 ng/well (saturating) recombinant TNFα. τ indicates the unimpeded binding of TNFα to STNFR2sig1. Controls are wells that did not receive TNFα (0 TNF) and wells coated with 100 ng Tmem273-Fc fusion protein. The screening assay identified sTNFR2-26C9 as a blocking antibody. FIG. 8B: sTNFR26C9 compared to non-blocking sTNFR2-1G2 antibody over a range of TNFα concentrations. % inhibition of TNFα binding indicated on the graph.

FIGS. 9A-9B depict the fragmentation of 26C9 into Fab and F(ab′)2 and relative concentration of each using mouse IgG1 Fragmentation Kit (Pierce catalog #44980), in accordance with some embodiments. FIG. 9A: SDS-PAGE analysis of fragmentation on a 4-12% gradient gel. Lanes 1G2 and 26C9 are purified antibodies. Flo thru is the fraction of 26C9 protein lacking a functional Fc domain that was not bound by the protein A column including Fab and F(ab′)2 fragments and a modest amount of contaminating intact 26C9. Elu are the 26C9 protein fragments eluted from the protein A column by low pH buffer including intact 26C9 antibody and partial digestion fragments that retain a function Fc domain. FIG. 9B: ImageJ densitometry quantifying individual bands in the purified Fab-F(ab′)2 preparation. % of total protein and % of antigen binding domain present in each fraction are indicated. 91% of the flo thru fraction used in subsequent experiments was either Fab or F(ab′)2 fragments with roughly an equal portion of each. Only 9% of the TNFR2 binding capacity was in the form of contaminating intact antibody.

FIG. 10 depicts the relative ability of 26C9 and 26C9 fragments to block TNFα binding to sTNFR2sig1 in the static assay, in accordance with some embodiments. The STNFR2-26C9 Fab-F(ab′)2 preparation was used in experiments to address whether the non-Fc fragments were capable of blocking TNFα binding to sTNFR2sig1. Wells were coated with 100 ng of sTNFRsig1, then pre-incubated with intact 26C9 (1.5 μg/well) or fragmented 26C9 (0.75 μg/well) prior to exposure to TNFα. Fragmented sTNFR2-26C9 was equally potent as an inhibitor as the intact antibody in the static assay.

FIG. 11 depicts the relative ability of sTNFR2-26C9 and 26C9 fragments to block PBMC IFN-γ production triggered by CD3 & CD28 in the dynamic assay, in accordance with some embodiments. Purified PBMC at 7.5×10⁶ were transiently exposed to monoclonal antibodies specific for CD3 & CD28 (CD3/28; 200 ng/ml) to provide a TCR activation signal, without and with intact sTNFR2-26C9 (CD3/28/26; 1.5 μg/ml), providing simultaneous transient blocking of TNFR2. Separately, transiently CD3/28 activated PBMC were added to wells containing fragmented 26C9 (3/28/Fab; 0.75 μg/well). Culture media contained recombinant human IL-2 (75 units/ml) and IL-7 (5 ng/ml). Supernatants were collected on day 3 and IFN-γ quantified as a measure of costimulatory activity. Cytokine=PBMC that did not receive a TCR activation stimulus. Statistical comparisons with a two-side Students t-test were performed as comparisons with the CD3/28 activated PBMC. **=p value <0.005; ****=p value <0.00005. Values in ( ) indicate % inhibition of costimulatory activity (i.e., T-cell activation).

FIG. 12 shows an CD3 (y-axis) vs CD4 (x-axis) staining from an unrelated sorting experiment based on the dynamic assay demonstrating normal levels of stainable cell surface CD3 on T cells after three days in culture under the conditions in FIG. 11, in accordance with some embodiments.

FIGS. 13A-13B show the conversion of rat IgG1 to human IgG4, in accordance with some embodiments. Using PROMETHEUS™ antibody humanization platform of Absolute Antibody LTD, the light and heavy chain backbones surrounding the 26C9 complement determining regions (CDR) were humanized, which produced a recombinant 26C9 of human IgG4 subclass. This humanized version of 26C9 is designated h26C9 herein. FIG. 13A: The human IgG4 subclass is known to spontaneously dissociated into monomers by cis disulfide bond formation. The formation of monomeric antibody in h26C9 was confirmed by the presence of antibody monomers (indicated by * in the NR lane) visible in the non-reduced state. Only heavy and light chains can be seen in the reduced gel (R lane). FIG. 13B: h26C9 retains potent inhibition of TNFα binding to sTNFR2 of the parent rat monoclonal antibody 26C9 in the static assay, >98% inhibition with p values <0.001 for comparisons with No Ab (no antibody) and 1G2 (monoclonal specific for sTNFR2 without blocking activity).

FIGS. 14A-14B show h26C9 stabilized with targeted mutations, in accordance with some embodiments. The targeted mutation resulted in the antibody which is designated as d26C9 herein. Specifically, using h26C9 as the base recombinant antibody, three specific modifications (described in Example 6 section in more details) were engineered into h26C9 to create d26C9. FIG. 14A: The stabilized recombinant antibody d26C9 does not spontaneously dissociate into monomers, as shown by the lack of a monomeric antibody band in the Non-reduced (NR) lane. FIG. 14B: d26C9 retains potent inhibition of TNFα binding to STNFR2, >98% inhibition, with p values <0.001 for comparisons with the 1G2 (monoclonal specific for sTNFR2 without blocking activity).

FIG. 15: structure of IgG4 and a non-limiting example of a minibody according to some embodiments. A minibody refers to a smaller version of an antibody having one or more non-antigen binding domains removed and is not limited to the exemplary minibody lacking CH2 domains found in IgG4 shown here. Other variations of minibodies can lack CH1, CL domains, and/or other non-antigen binding domains.

FIG. 16 demonstrate that d26C9 is both humanized and capable of binding antigen, in accordance with some embodiments. Nunc maxisorp ELISA plates were coated ON with 50 μl PBS containing 2 μg/ml sTNFR2-sig1. Rat monoclonals 1G2 and 26C9 were not detected by anti-human Fc gamma-HRP. Humanized/stabilized d26C9 IgG4 bound to sTNFR2 was detected by anti-human Fc gamma-HRP confirming its humanization and binding to sTNFR2 by a direct independent method.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Currently, major human clinical investigations measure human sTNFR2 levels using R & D Systems ELISA kits. Such kits comprise a goat anti-human sTNFR2 polyclonal antiserum in the detection step. Polyclonal antibodies are less desirable than monoclonal antibodies in quantifications, as polyclonal antibodies are intrinsically finite reagents recognizing multiple epitopes including potentially the epitope recognized by the capture antibody. Furthermore, the current R & D systems kit uses a recombinant sTNFR2 fragment (aa24-206) that is denatured by method of generation in E. coli, whose 183 aa (19,482 daltons) are 77% by mass of the extracellular domain of TNFR2 (235aa, 25,131 daltons), and is of unknown relationship to the biologically relevant Adams 17-processed form of sTNFR2.

To address the lack of CLIA-certifiable ELISA reagents for soluble TNFR2, the study described herein (“the present study”) designed fusion proteins having N-terminal stabilized sTNFR2 extracellular domain linked to rat IgG2b, and utilized rats rather than mice to generate a new spectrum of anti-human sTNFR2 monoclonal antibodies that could function as capture-detection pairs in devices capable of standardization and reproducible measurement of sTNFR2.

Non-limiting examples of the antibodies generated in the present study were evaluated and confirmed to be highly specific for sTNFR2 and not cross-react with sTNFR1.

Accordingly, in some aspects, the present invention is directed to an antibody. In some embodiments, the antibody targets tumor necrosis factor receptor 2 (TNFR2). In some embodiments, the antibody targets the extracellular portion of TNFR2. In some embodiments, the antibody targets the soluble TNFR2 (sTNFR2), such as the sTNRF2 generated by protease digestion of membrane bound TNFR2, such as the Adams17-processed form of STNFR2. In some embodiments, the TNFR2 or sTNFR2 is human TNFR2 or human STNFR2.

In some aspects, the present invention is directed to a nucleic acid encoding an antibody, such as the antibody described herein.

The present study further confirmed that non-limiting examples of antibodies herein, when used in capture-detection pairs, are able to capture sTNFR2 in samples and quantify the captured sTNFR2 with high sensitivity and specificity.

Accordingly, in some aspects, the present invention is directed to an antibody pair. In some embodiments, the antibody pair captures sTNFR2 in a sample and detect the captured STNFR2, thereby quantifying the sTNFR2 in the sample.

In some aspects, the present invention is directed to a method of quantifying sTNFR2 in samples using the antibody pair. In some embodiments, quantifying sTNFR2 in samples diagnoses diseases or disorders (such as Kawasaki disease or Kawasaki-like syndrome), or predicts outcomes of the disease or disorder or monitors the state of the disease or disorder during therapies.

The effective diagnosis and treatment of Kawasaki disease (K_{D}) or Kawasaki-like syndrome (KLS) is hindered by the lack of quality sTNFR2 antibodies and are expected to be highly effective if such antibodies are available. The present invention provides such quality sTNF2 antibodies.

Accordingly, in some aspects, the present invention is directed to a method of diagnosing Kawasaki disease (K_{D}) or Kawasaki-like syndrome (KLS).

Accordingly, in some aspects, the present invention is directed to a method of treating, ameliorating, and/or preventing Kawasaki disease (K_{D}) or Kawasaki-like syndrome (KLS).

TNF alpha (TNFα) and lymphotoxin alpha (Ltα) bind two related receptors, TNFR1 and TNFR2, with differing biologic activities that are central to many inflammatory pathways and diseases. In general terms, binding and activation of TNFR1 mediates effector functions of TNFα including antimicrobial effects through the classical NFkB pathway activation of cell intrinsic and extrinsic innate defense mechanisms and anti-tumor effects via death domain recruitment of TRADD linked to activated cell death pathways. Conversely, binding and activation of TNFR2 augments T cell activation and regulation with additional effects on nonlymphoid cell types. TNFR1 is ubiquitously expressed while TNFR2 is limited to lymphocytes including T cells and a limited set of non-hematopoetic cell populations including endothelial cells. One of the major organs affected by TNFα is the circulatory system when TNFα causes vasodilatation, leukocyte adhesion and increased coagulability. Current anti-TNFα therapies used in clinical practice are limited to binding TNFα and thereby preventing TNFα interaction with either receptor. Global dampening of TNFα biologic activity would predictably have negative effects on controlling microbial infections and tumor surveillance. Those theoretical adverse events of infection and malignancy are borne out as known risks associated with the use of TNFα blockers in clinical practice. TNFα blockade therapeutics including, Remicade®, Enbel®, and Humira®, have FDA black box warnings regarding increased susceptibility to infection and risk of malignancies, especially lymphoma (~3-fold). Murine models of Listeria monocytogenes demonstrate the functional divide between TNFR1 and TNFR2. TNFR1 knockout (ko) mice succumb to listeria infections while TNFR2 mice have prolonged infections but ultimately clear the bacterium and survive. Importantly in the latter study, TNFR1 ko, and TNFα ko mice (analogous to pan TNFα blockade), succumbed to listeria infection.

While it is generally understood that TNFα blockades' beneficial effects are mediated through dampening of TNFR1-mediated inflammation, that data comes from collagen-induced arthritis model that has an unclear relationship with rheumatoid arthritis pathophysiology. In certain non-limiting embodiments, blockade of TNFR1 signaling in inflammatory processes that have a microbial infection component may be detrimental, possibly explaining the failure of TNFα blockade strategies in sepsis. Oncologic interventions based on tumor expression of TNFR2, and depletion of TNFR2 dependent Treg to activate tumor infiltrated lymphocytes (TIL), are a major focus selective TNFR2 blockade. However, it is well-established that TNFα binding to TNFR2 is costimulatory for naïve T cells and proinflammatory in effector T cells. Therefore, TNFR2 signaling is a potential target for managing autoimmune diseases and the tempering cytokine storm and hypercoagulability that contributes to mortality during severe infections and autoimmune diseases. TNFα signaling through TNFR2 is necessary for CD4 and CD8 T cell expansion, survival of effector T cells, and development of long-term memory in the Listeria monocytogenes mouse model. Tempering TNFα signaling through TNFR2 theoretically can dampen T cell driven inflammation and vascular pathology without grossly compromising innate antimicrobial defenses and cancer surveillance.

In order to selectively block TNFR2 signaling, agents are needed that prevent binding of TNFα (and Ltα) to the TNFR2 without triggering the receptor. Among potential inhibitory agents are antibodies. In one aspect, the disclosure provides a TNFR2 Fc fusion protein that binds TNFα in a static in vitro assay. In one aspect, the disclosure provides a dynamic in vitro assay based on PBMC for assessing costimulatory activity, such as T-cell activation. In one aspect, the disclosure provides a monoclonal antibody that blocks TNFα binding to TNFR2 in the static assay and TNER2 costimulatory activity (e.g., T-cell activation) in the dynamic assay.

### DEFINITIONS

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

The term “antibody” or “Ab” or “immunoglobulin” are terms of art and can be used interchangeably and refer to a protein, or polypeptide sequence which is or is derived from an immunoglobulin molecule having at least one antigen binding site which specifically binds to a specific epitope on an antigen (See, e.g., Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, hetero-conjugate antibodies, single domain antibodies, monovalent antibodies, single chain antibodies or single chain Fv (scFv), nanobodies, intracellular antibodies, intrabodies, camelized antibodies, camelid antibodies, IgNAR antibodies, affybodies, Fab fragments, F(ab′) fragments, F (ab) 2, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. Full-length antibodies are typically tetramers comprising two heavy chain and two light chain immunoglobulin molecules. The term “antibody” as used herein generally include fragments of full-length antibodies that are able to bind to antigens. See the definition of terms “antibody fragment,” “antigen-binding fragment,” and “antigen-binding domain” for definition of such fragments.

As used herein, the term “monoclonal antibody” refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., a hybridoma or host cell producing a recombinant antibody), wherein the antibody binds to a TNFR2 protein epitope as determined, e.g., by ELISA or other antigen-binding or competitive binding assay known in the art or in the Examples provided herein. In particular embodiments, a monoclonal antibody can be a chimeric antibody, a human antibody, or a humanized antibody. Methods for generating a humanized antibody are known in the art. In certain embodiments, a monoclonal antibody is a monovalent antibody or multivalent (e.g., bivalent) antibody. In particular embodiments, a monoclonal antibody is a monospecific or multi-specific antibody (e.g., bispecific antibody). Monoclonal antibodies described herein can, for example, can be made by the hybridoma method as described in Kohler et al.; Nature, 256:495 (1975) or can be isolated from phage libraries, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Monoclonal antibodies may be identified by high-throughput direct sequencing of fully recombined VDJ sequences of B cell receptor (BCR) repertoires from single cells of animals immunized with an antigen for which the desired monoclonal antibody will specifically bind as described herein. See, e.g. Goldstein et al., Communications Biology (2019) 2:304; Horns et al., Cell Reports (2020) 30:905-913). The identified monoclonal antibodies are then produced recombinantly.

The terms “antibody fragment,” “antigen-binding fragment,” and “antigen-binding domain” of an antibody and similar terms are used interchangeably and refer to at least one portion of an intact antibody, or recombinant variants thereof, and comprising or consisting of the antigen-binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), VHH domains, and multi-specific (e.g., bispecific) antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

As used herein, the term “constant region” or “constant domain” refers to an antibody portion, e.g., a carboxyl terminal portion of a light and/or heavy chain which is not directly involved in binding of an antibody to antigen but which can exhibit various effector functions, such as interaction with the Fe receptor. The terms refer to a portion of an immunoglobulin molecule having a generally more conserved amino acid sequence relative to an immunoglobulin variable domain.

As used herein, the terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs).

By the term “recombinant antibody” as used herein, is meant an antibody which is generated using recombinant DNA technology, such as, for example, an antibody expressed by a bacteriophage, insect, or yeast expression system or by a mammalian cell expression system, such as a human cell line expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. It is readily apparent that an antigen can be generated, synthesized, or can be derived from a biological sample. Such a biological sample can include, but is not limited to a tissue sample, a tumor sample, a cell, a virus, or a biological fluid.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope can be determined by structural methods, e.g., X-ray diffraction crystallography, nuclear magnetic resonance (NMR), or electron microscopy (e.g., negative stain or cryo-EM), ELISA assays, hydrogen/deuterium exchange coupled with mass spectrometry (e.g., MALDI mass spectrometry), array-based oligo-peptide scanning assays, and/or mutagenesis mapping (e.g., site-directed mutagenesis mapping). In a specific embodiment, the epitope of an antibody or antigen-binding fragment is determined using cryo-EM studies, such as described herein.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (K_{D}). Affinity can be measured and/or expressed in a number of ways known in the art, including, but not limited to, equilibrium dissociation constant (K_{D}), equilibrium association constant (K_{A}), and IC50. The K_{D} is calculated from the quotient of k_{off}/kₒₙ, whereas K_{A} is calculated from the quotient of kₒₙ/k_{off}, where kₒₙ refers to the association rate constant of, e.g., an antibody to an antigen, and k_{off} refers to the dissociation rate constant of, e.g., an antibody to an antigen.

The term “avidity” as used herein refers to the total binding strength of an antibody for an antigen at every binding site.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lenti viruses, retroviruses, adenoviruses, and adeno-associated viruses) that incorporate the recombinant polynucleotide.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A “condition” is a state of health, whether well or ill, and may include, for example, the state of having an infection (e.g., a viral, bacterial, or fungal infection) whether or not the animal has noticeable or detectable symptoms of the infection.

A disease, disorder, or condition is “alleviated” or “ameliorated” if the severity or frequency of at least one sign or symptom of the disease or disorder experienced by a subject is reduced and/or eliminated.

The terms “treat,” “treating,” and “treatment,” refer to one or more therapeutic or palliative measures described herein. The methods of “treatment” employ administration of a composition to a subject in need of such treatment, for example, a subject afflicted with a disease or disorder or condition, or a subject who has one or more signs or symptoms of such a disease or disorder or condition, in order to cure, delay, reduce the severity of, alleviate, or ameliorate one or more signs or symptoms of the disease, disorder or condition, or in order to prolong the survival of a subject beyond that expected in the absence of such treatment.

As used herein, the terms “peptide,” “polypeptide,” and “protein” are used interchangeably, and refer to a compound comprised of amino acid residues covalently linked by peptide bonds, having an N-terminus and a C-terminus. A protein or peptide must contain at least two amino acids, and no limitation is placed on the maximum number of amino acids that may comprise a protein or peptide's sequence. Polypeptides include any peptide comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. Proteins may comprise more than one polypeptide chain. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

The terms “pharmaceutically effective amount” and “effective amount” refer to a non-toxic but sufficient amount of an agent to provide the desired biological result. That result can be reduction and/or alleviation of the signs, symptoms, or causes of a disease or disorder, or any other desired alteration of a biological system.

By the term “specifically binds” as used herein, is meant a molecule, such as an antibody, which recognizes and binds to another molecule or feature, but does not substantially recognize or bind other molecules or features in a sample.

The terms “subject” or “patient” or “individual” for the purposes of the present disclosure includes humans and other animals, particularly mammals, and other organisms. Non-human mammals include, for example, livestock and pets, non-human simian, ovine, bovine, porcine, canine, feline and murine mammals. Preferably, the subject is human. Thus the methods are applicable to both human therapy and veterinary applications.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Antibodies

In some aspects, the instant specification is directed to an antibody. In some embodiments, the antibody is an antibody targeting tumor necrosis factor receptor 2 (TNFR2). In some embodiments, the antibody is an antibody targeting an extracellular portion of TNFR2. In some embodiments, the antibody is an antibody targeting membrane bound TNFR2. In some embodiments, the antibody is an antibody targeting soluble TNFR2 (STNFR2). In some embodiments, the antibody does not cross-react with sTNFR1. In some embodiments, the TNFR2 and/or the sTNFR2 are human TNFR2 and/or human sTNFR2. In some embodiments, the antibody blocks or reduces an interaction between TNFR2 and the ligand TNFα. In some embodiments, the antibody is an antibody that reduces the production of IFN-γ by a cell, such as by the peripheral blood mononuclear cells (PBMCs).

In certain embodiments, the antibody is isolated.

In certain embodiments, the antibody is recombinant.

In certain embodiments, the antibody is an IgG, IgM, IgA, or an antigen binding fragment thereof.

In certain embodiments, the antibody is a Fab′, a F(ab′)2, a F(ab′)3, a monovalent scFv, a bivalent scFv, or a single domain antibody.

In certain embodiments, the antibody is a full length antibody.

In certain embodiments, the antibody is a human, humanized antibody, or de-immunized antibody.

In certain embodiments, the antibody can be conjugated to an imaging agent, a chemotherapeutic agent, a toxin, and/or a radionuclide.

In some embodiments, the antibody includes a heavy chain polypeptide and a light chain polypeptide.

In some embodiments, the heavy chain polypeptide includes one or more of the following:
(a) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 1; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:2; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:3;
(b) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:7; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:8; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:9;
(c) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 13; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 14; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:15;
(d) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:18; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 19; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:20;
(e) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:23; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:24; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:25.

In some embodiments, the light chain polypeptide includes one or more of the following:
(a) CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 4; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:5; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:6;
(b) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 10; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 11; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO: 12;
(c) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 16; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:17;
(d) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:21; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:22;
(e) a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:26; a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:27; and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:28.

In some embodiments, the antibody includes one or more of the following:
(a) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:1, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:2, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:3; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:5, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:6;
(b) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:7, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:8, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:9; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 10, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 11, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO: 12;
(c) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 13, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:14, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:15; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 16, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:17;
(d) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 18, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:19, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:20; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:21, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:22;
(e) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:23, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:24, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:25; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:26, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:27, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:28.

TABLE 1
CDRs of the exemplary antigen binding proteins
1. Clone 1G2
HCDR1: GFTFSTA (SEQ ID NO: 1)
HCDR2: QIKAKSNNYA (SEQ ID NO: 2)
HCDR3: YCTGGNSVDY (SEQ ID NO: 3)
VCDR1: QNINKF (SEQ ID NO: 4)
VCDR2: NTN (SEQ ID NO: 5)
VCDR3: LQHIRFPGT (SEQ ID NO: 6)
2. Clone 6D7
HCDR1: GYTFTDH (SEQ ID NO: 7)
HCDR2: EISPNYDT (SEQ ID NO: 8)
HCDR3: YCSRTNSGWYF (SEQ ID NO: 9)
VCDR1: SSVRN (SEQ ID NO: 10)
VCDR2: STS (SEQ ID NO: 11)
VCDR3: QQWSRNPPT (SEQ ID NO: 12)
3. Clone 9C10
HCDR1: GFTFSNA (SEQ ID NO: 13)
HCDR2: QIKAKSNNFA (SEQ ID NO: 14)
HCDR3: FCAGGNTWDY (SEQ ID NO: 15)
VCDR1: QNINKF (SEQ ID NO: 4)
VCDR2: SPN (SEQ ID NO: 16)
VCDR3: LQHASFPGT (SEQ ID NO: 17)
4. Clone 26C9
HCDR1: GFSLSTYGV (SEQ ID NO: 18)
HCDR2: NIWWDDD (SEQ ID NO: 19)
HCDR3: YCARIDGTRYF (SEQ ID NO: 20)
VCDR1: QNINKF (SEQ ID NO: 4)
VCDR2: YTS (SEQ ID NO: 21)
VCDR3: LQYGNLWT (SEQ ID NO: 22)
5. Clone 30C5
HCDR1: GYIFTSY (SEQ ID NO: 23)
HCDR2: YINTGNGG (SEQ ID NO: 24)
HCDR3: YCARGHPYYGYYL (SEQ ID NO: 25)
VCDR1: QDIGNW (SEQ ID NO: 26)
VCDR2: GAT (SEQ ID NO: 27)
VCDR3: LQTYTSPWT (SEQ ID NO: 28)

In some embodiments, the heavy chain polypeptide includes at least one of the following:
(a) a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:29;
(b) a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:31;
(c) a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:33,
(d) a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:35,
(e) a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:37.

In some embodiments, the light chain polypeptide includes at least one of the following:
(a) a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:30;
(b) a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:32;
(c) a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:34.
(d) a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:36;

(e) a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:38.

In some embodiments the antibody includes one or more of the following:
(a) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:29; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:30;
(b) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:31; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:32;
(c) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:33; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:34;
(d) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:35; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:36;
(e) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:37; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:38;
(f) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:87; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:88.

TABLE 2
Variable regions of the exemplary antigen binding proteins
1. Clone 1G2
Heavy Chain Variable Region (V_{H}):
QVQLKESGGSLVQPGKSLKLTCVTSGFTFSTAWMHWVRQSPEKQLEWVAQIKAK
SNNYATYYAESVKGRFTISRDDSKNSVYLQMNSLKEEDTAIYYCTGGNSVDYWG
QGVMVTVSS (SEQ ID NO: 29)
Light Chain Variable Region (V_{L}):
NIVLTQSPSFLPASVGDRVTINCKASQNINKFLNWYQQKLGEAPKRLIYNTNNLDT
GTPSRFSGSGSGTDYTLTISSLQPEDFATYFCLQHIRFPGTFGGGTMLELR (SEQ ID
NO: 30)
2. Clone 6D7
Heavy Chain Variable Region (V_{H}):
QVQLKESGAALVKPGASVKMSCKASGYTFTDHWVSWVRQSHGRSLEWIGEISPN
YDTTNFNEKFRGRATLTVDKSTSTASMELSRLTSEDSAIYYCSRTNSGWYFDFWG
PGTMVTVSS (SEQ ID NO: 31)
Light Chain Variable Region (V_{L}):
NIVMTQSPTTTAASPGEKVTITCLASSSVRNIYWYQQKSGASPKPLIYSTSSLASGV
PDRFSGSGSGTSYSLTINTMEAEDAATYYCQQWSRNPPTFGSGTKLEIK (SEQ ID
NO: 32)
3. Clone 9C10
Heavy Chain Variable Region (V_{H}):
QVQLKESGGSLVQPGKSLKLTCATSGFTFSNAWMHWVRQSPEKQLEWVAQIKAK
SNNFATYYAESVRGRFIISRDDSKSTVYLQMNNLREEDTAIYFCAGGNTWDYWGQ
GVMVTVSS (SEQ ID NO: 33)
Light Chain Variable Region (V_{L}):
AAANSPLIGAPGGAEGDRVTINCTASQNINKFLNWYQQRLGKAPKRLILSPNNLQA
GVPSRFSGSGSGTDYTLTISNLQPEDFASYFCLQHASFPGTFGGGTKLELI (SEQ ID
NO: 34)
4. Clone 26C9
Heavy Chain Variable Region (V_{H}):
QVQLKESGPGILQPSQTLSLTCTFSGFSLSTYGVGVGWIRQPSGKGLEWLANIWWD
DDKYYNPSLKNRLTISKDTSNNQVFLKITNVDTADTATYYCARIDGTRYFDYWGQ
GVMVSVSS (SEQ ID NO: 35)
Light Chain Variable Region (V_{L}):
DIVLTQSPPSLSASLGDKVTITCQTSQNINKFIAWYQQKPGKAPRQLIRYTSTLMSG
TPSRFSGSGSGTDYSFSISNVESEDIATYYCLQYGNLWTFGRGTKLELK (SEQ ID
NO: 36)
5. Clone 30C5
Heavy Chain Variable Region (V_{H}):
QVQLTESGAELAKPGSSVKISCKASGYIFTSYYISWIKQTTGQGLDYVGYINTGNG
GTHYSERFKDSATLTVDRSSSTAFMQLSSLTPDDSAVYYCARGHPYYGYYLDYW
GQGVMVTVSS (SEQ ID NO: 37)
Light Chain Variable Region (V_{L}):
DIVLTQSPASLSASLEEIVTITCQASQDIGNWLSWYQQSPGKSPQLLIYGATSLADG
VPSRFSGSRSGTQFSLTISRLQVEDIGIYYCLQTYTSPWTFGGGTKLELK (SEQ ID
NO: 38)
6. h26C9 and d26C9
Heavy Chain Variable Region (V_{H}):
QVQLKESGPGILQPSQTLSLTCTFSGFSLSTYGVGVGWIRQPSGKGLEWLANIWWD
DDKYYNPSLKNRLTISKDTSNNQVFLKITNVDTADTATYYCARIDGTRYFDYWGQ
GVMVSVSS (SEQ ID NO: 87)
Light Chain Variable Region (V_{L}):
DIVLTQSPPSLSASLGDKVTITCQTSQNINKFIAWYQQKPGKAPRQLIRYTSTLMSG
TPSRFSGSGSGTDYSFSISNVESEDIATYYCLQYGNLWTFGRGTKLEIK (SEQ ID
NO: 88)

Nucleic Acid that Encodes Antibody

In some aspects, the present invention is directed to a nucleic acid that encodes an antibody. In some embodiments, the antibody is the same as or similar to those as described elsewhere herein, such as in the “Antibody” section.

In some embodiments, the nucleic acid includes a first nucleic acid. In some embodiments, the first nucleic acid encodes a heavy chain variable region.

In some embodiments the first nucleic acid including one or more of the following:
(a) a first portion encoding an amino acid sequence of SEQ ID NO:1; a second portion encoding an amino acid sequence of SEQ ID NO:2; and a third portion encoding an amino acid sequence of SEQ ID NO:3;
(b) a first portion encoding an amino acid sequence of SEQ ID NO:7; a second portion encoding an amino acid sequence of SEQ ID NO:8; and a third portion encoding an amino acid sequence of SEQ ID NO:9;
(c) a first portion encoding an amino acid sequence of SEQ ID NO:13; a second portion encoding an amino acid sequence of SEQ ID NO:14; and a third portion encoding an amino acid sequence of SEQ ID NO:15;
(e) a first portion encoding an amino acid sequence of SEQ ID NO:18; a second portion encoding an amino acid sequence of SEQ ID NO:19; and a third portion encoding an amino acid sequence of SEQ ID NO:20;
(f) a first portion encoding an amino acid sequence of SEQ ID NO:23; a second portion encoding an amino acid sequence of SEQ ID NO:24; and a third portion encoding an amino acid sequence of SEQ ID NO:25.

In some embodiments, the nucleic acid includes a second nucleic acid. In some embodiments, the second nucleic acid encodes a light chain polypeptide. In some embodiments, the second nucleic acid includes one or more of the following:
(a) a first portion encoding an amino acid sequence of SEQ ID NO:4; a second portion encoding an amino acid sequence of SEQ ID NO:5; and a third portion encoding an amino acid sequence of SEQ ID NO:6;
(b) a first portion encoding an amino acid sequence of SEQ ID NO:10; a second portion encoding an amino acid sequence of SEQ ID NO:11; and a third portion encoding an amino acid sequence of SEQ ID NO:12;
(c) a first portion encoding an amino acid sequence of SEQ ID NO:4; a second portion encoding an amino acid sequence of SEQ ID NO:16; and a third portion encoding an amino acid sequence of SEQ ID NO:17;
(d) a first portion encoding an amino acid sequence of SEQ ID NO:4; a second portion encoding an amino acid sequence of SEQ ID NO:21; and a third portion encoding an amino acid sequence of SEQ ID NO:22;
(e) a first portion encoding an amino acid sequence of SEQ ID NO:26; a second portion encoding an amino acid sequence of SEQ ID NO:27; and a third portion encoding an amino acid sequence of SEQ ID NO:28.

In some embodiments, the first nucleic acid includes one or more of the following:
(a) a first portion including the sequence of SEQ ID NO:39; a second portion including the sequence of SEQ ID NO:40; and a third portion including the sequence of SEQ ID NO: 41;
(b) a first portion including the sequence of SEQ ID NO:45; a second portion including the sequence of SEQ ID NO:46; and a third portion including a sequence of SEQ ID NO: 47;
(c) a first portion including the sequence of SEQ ID NO:51; a second portion including the sequence of SEQ ID NO:52; and a third portion including the sequence of SEQ ID NO: 53;
(d) a first portion including the sequence of SEQ ID NO:56; a second portion including the sequence of SEQ ID NO:57; and a third portion including a sequence of SEQ ID NO: 58;
(e) a first portion including the sequence of SEQ ID NO:62; a second portion including the sequence of SEQ ID NO:63; and a third portion including the sequence of SEQ ID NO: 64.

In some embodiments, the second nucleic acid includes one or more of the following:
(a) a first portion including the sequence of SEQ ID NO:42; a second portion including the sequence of SEQ ID NO:43; and a third portion including the sequence of SEQ ID NO: 44;
(b) a first portion including the sequence of SEQ ID NO:48; a second portion including the sequence of SEQ ID NO:49; and a third portion including a sequence of SEQ ID NO: 50;
(c) a first portion including the sequence of SEQ ID NO:42; a second portion including the sequence of SEQ ID NO:54; and a third portion including the sequence of SEQ ID NO: 55;
(d) a first portion including the sequence of SEQ ID NO:59; a second portion including the sequence of SEQ ID NO:60; and a third portion including a sequence of SEQ ID NO: 61;
(e) a first portion including the sequence of SEQ ID NO:65; a second portion including the sequence of SEQ ID NO:66; and a third portion including the sequence of SEQ ID NO: 67.

In some embodiments, the nucleic acid includes at least one of the following:
(a) a first nucleic acid including a first portion including the sequence of SEQ ID NO: 39, a second portion including the sequence of SEQ ID NO:40, and a third portion including the sequence of SEQ ID NO:41; and a second nucleic acid including a first portion including the sequence of SEQ ID NO:42, a second portion including the sequence of SEQ ID NO: 43, and a third portion including the sequence of SEQ ID NO:44;
(b) a first nucleic acid including a first portion including the sequence of SEQ ID NO: 45, a second portion including the sequence of SEQ ID NO:46, and a third portion including the sequence of SEQ ID NO:47; and a second nucleic acid including a first portion including the sequence of SEQ ID NO:48 a second portion including the sequence of SEQ ID NO: 48, and a third portion including the sequence of SEQ ID NO:50;
(c) a first nucleic acid including a first portion including the sequence of SEQ ID NO: 51, a second portion including the sequence of SEQ ID NO:52, and a third portion including the sequence of SEQ ID NO:53; and a second nucleic acid including a first portion including the sequence of SEQ ID NO:42 a second portion including the sequence of SEQ ID NO: 54, and a third portion including the sequence of SEQ ID NO:55;
(d) a first nucleic acid including a first portion including the sequence of SEQ ID NO: 56, a second portion including the sequence of SEQ ID NO:57, and a third portion including the sequence of SEQ ID NO:58; and a second nucleic acid including a first portion including the sequence of SEQ ID NO:59 a second portion including the sequence of SEQ ID NO: 60, and a third portion including the sequence of SEQ ID NO:61;
(e) a first nucleic acid including a first portion including the sequence of SEQ ID NO: 62, a second portion including the sequence of SEQ ID NO:63, and a third portion including the sequence of SEQ ID NO:64; and a second nucleic acid including a first portion including the sequence of SEQ ID NO:65 a second portion including the sequence of SEQ ID NO: 66, and a third portion including the sequence of SEQ ID NO:67.

TABLE 3
Nucleic acid sequences encoding CDRs of the exemplary antigen binding
proteins
1. Clone 1G2
HCDR1: GGATTCACTTTCAGTACCGCC (SEQ ID NO: 39)
HCDR2: CAAATTAAAGCCAAATCAAACAATTATGCA (SEQ ID NO: 40)
HCDR3: TATTGTACGGGAGGGAATAGTGTTGATTAC (SEQ ID NO: 41)
VCDR1: CAGAATATTAACAAGTTC (SEQ ID NO: 42)
VCDR2: AATACAAAC (SEQ ID NO: 43)
VCDR3: TTACAGCATATTCGTTTTCCCGGGACG (SEQ ID NO: 44)
2. Clone 6D7
HCDR1: GGTTATACATTCACTGACCAC (SEQ ID NO: 45)
HCDR2: GAAATATCTCCTAACTACGATACT (SEQ ID NO: 46)
HCDR3: TACTGTTCAAGAACGAACTCAGGCTGGTACTTT (SEQ ID NO: 47)
VCDR1: TCCAGTGTAAGGAAC (SEQ ID NO: 48)
VCDR2: TCCACATCG (SEQ ID NO: 49)
VCDR3: CAGCAGTGGAGTAGGAACCCACCTACG (SEQ ID NO: 50)
3. Clone 9C10
HCDR1: GGATTCACTTTCAGTAACGCC (SEQ ID NO: 51)
HCDR2: CAAATTAAAGCCAAATCAAACAATTTTGCA (SEQ ID NO: 52)
HCDR3: TTCTGTGCCGGCGGTAACACCTGGGATTAC (SEQ ID NO: 53)
VCDR1: CAGAATATTAACAAGTTC (SEQ ID NO: 42)
VCDR2: AGTCCAAAC (SEQ ID NO: 54)
VCDR3: TTGCAGCATGCTAGTTTTCCCGGGACG (SEQ ID NO: 55)
4. Clone 26C9
HCDR1: TCTGGGTTTTCACTGAGCACTTATGGTGTG (SEQ ID NO: 56)
HCDR2: AACATTTGGTGGGATGATGAT (SEQ ID NO: 57)
HCDR3: TACTGTGCTCGGATAGATGGTACCCGGTACTTT (SEQ ID NO: 58)
VCDR1: CAAAACATTAACAAGTTT (SEQ ID NO: 59)
VCDR2: TACACTTCT (SEQ ID NO: 60)
VCDR3: CTACAGTACGGTAACCTGTGGACG (SEQ ID NO: 61)
5. Clone 30C5
HCDR1: GGCTACATTTTCACCAGCTAC (SEQ ID NO: 62)
HCDR2: TATATTAATACGGGAAATGGAGGT (SEQ ID NO: 63)
HCDR3: TACTGTGCAAGAGGCCATCCATACTATGGATATTACCTT (SEQ ID NO: 64)
VCDR1: CAGGACATCGGTAATTGG (SEQ ID NO: 65)
VCDR2: GGTGCAACC (SEQ ID NO: 66)
VCDR3: CTACAGACTTATACTAGTCCGTGGACG (SEQ ID NO: 67)

In some embodiments, the first nucleic acid includes at least one of the following:
(a) a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:29;
(b) a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:31;
(c) a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:33;
(d) a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:35;
(e) a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:37.

In some embodiments, the second nucleic acid includes at least one of the following:
(a) a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:30;
(b) a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:32;
(c) a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:34,
(d) a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:36;
(e) a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:38.

In some embodiments, the nucleic acid includes at least one of the following:
(a) a first nucleic acid including a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:29; and a second nucleic acid including a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:30;
(b) a first nucleic acid including a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:31; and a second nucleic acid including a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:32;
(c) a first nucleic acid including a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:33; and a second nucleic acid including a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:34;
(d) a first nucleic acid including a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:35; and a second nucleic acid including a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:36;
(e) a first nucleic acid including a nucleic acid encoding a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:37; and a second nucleic acid including a nucleic acid encoding a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:38.

In some embodiments, the first nucleic acid includes at least one of the following:
(a) a nucleic acid including a sequence that is about 80%, 85%, 90%, 91%, 92%, 93%, 94%, 95%, 96%, 97%, 98%, 99% or 100% identical to the sequence of SEQ ID NO:68;
(b) a nucleic acid including a sequence that is about 80%, 85%, 90%, 91%, 92%, 93%, 94%, 95%, 96%, 97%, 98%, 99% or 100% identical to the sequence of SEQ ID NO:70;
(c) a nucleic acid including a sequence that is about 80%, 85%, 90%, 91%, 92%, 93%, 94%, 95%, 96%, 97%, 98%, 99% or 100% identical to the sequence of SEQ ID NO:72.
(d) a nucleic acid including a sequence that is about 80%, 85%, 90%, 91%, 92%, 93%, 94%, 95%, 96%, 97%, 98%, 99% or 100% identical to the sequence of SEQ ID NO:74;
(e) a nucleic acid including a sequence that is about 80%, 85%, 90%, 91%, 92%, 93%, 94%, 95%, 96%, 97%, 98%, 99% or 100% identical to the sequence of SEQ ID NO:76.

In some embodiments, the second nucleic acid includes at least one of the following:
(a) a nucleic acid including a sequence that is about 80%, 85%, 90%, 91%, 92%, 93%, 94%, 95%, 96%, 97%, 98%, 99% or 100% identical to the sequence of SEQ ID NO:69;
(b) a nucleic acid including a sequence that is about 80%, 85%, 90%, 91%, 92%, 93%, 94%, 95%, 96%, 97%, 98%, 99% or 100% identical to the sequence of SEQ ID NO:71;
(c) a nucleic acid including a sequence that is about 80%, 85%, 90%, 91%, 92%, 93%, 94%, 95%, 96%, 97%, 98%, 99% or 100% identical to the sequence of SEQ ID NO:73.
(d) a nucleic acid including a sequence that is about 80%, 85%, 90%, 91%, 92%, 93%, 94%, 95%, 96%, 97%, 98%, 99% or 100% identical to the sequence of SEQ ID NO:75;
(e) a nucleic acid including a sequence that is about 80%, 85%, 90%, 91%, 92%, 93%, 94%, 95%, 96%, 97%, 98%, 99% or 100% identical to the sequence of SEQ ID NO:77.

In some embodiments, the nucleic acid includes at least one of the following:
(a) a first nucleic acid including a sequence that is about 80%, 85%, 90%, 91%, 92%, 93%, 94%, 95%, 96%, 97%, 98%, 99% or 100% identical to the sequence of SEQ ID NO:68; and a second nucleic acid including a sequence that is about 80%, 85%, 90%, 91%, 92%, 93%, 94%, 95%, 96%, 97%, 98%, 99% or 100% identical to the sequence of SEQ ID NO:69;
(b) a first nucleic acid including a sequence that is about 80%, 85%, 90%, 91%, 92%, 93%, 94%, 95%, 96%, 97%, 98%, 99% or 100% identical to the sequence of SEQ ID NO:70; and a second nucleic acid including a sequence that is about 80%, 85%, 90%, 91%, 92%, 93%, 94%, 95%, 96%, 97%, 98%, 99% or 100% identical to the sequence of SEQ ID NO:71;
(c) a first nucleic acid including a sequence that is about 80%, 85%, 90%, 91%, 92%, 93%, 94%, 95%, 96%, 97%, 98%, 99% or 100% identical to the sequence of SEQ ID NO:72; and a second nucleic acid including a sequence that is about 80%, 85%, 90%, 91%, 92%, 93%, 94%, 95%, 96%, 97%, 98%, 99% or 100% identical to the sequence of SEQ ID NO:73;
(d) a first nucleic acid including a sequence that is about 80%, 85%, 90%, 91%, 92%, 93%, 94%, 95%, 96%, 97%, 98%, 99% or 100% identical to the sequence of SEQ ID NO:74; and a second nucleic acid including a sequence that is about 80%, 85%, 90%, 91%, 92%, 93%, 94%, 95%, 96%, 97%, 98%, 99% or 100% identical to the sequence of SEQ ID NO:75;
(e) a first nucleic acid including a sequence that is about 80%, 85%, 90%, 91%, 92%, 93%, 94%, 95%, 96%, 97%, 98%, 99% or 100% identical to the sequence of SEQ ID NO:76; and a second nucleic acid including a sequence that is about 80%, 85%, 90%, 91%, 92%, 93%, 94%, 95%, 96%, 97%, 98%, 99% or 100% identical to the sequence of SEQ ID NO:77;
(e) a first nucleic acid including a sequence that is about 80%, 85%, 90%, 91%, 92%, 93%, 94%, 95%, 96%, 97%, 98%, 99% or 100% identical to the sequence of SEQ ID NO:89; and a second nucleic acid including a sequence that is about 80%, 85%, 90%, 91%, 92%, 93%, 94%, 95%, 96%, 97%, 98%, 99% or 100% identical to the sequence of SEQ ID NO:90.

TABLE 4
Nucleic acid sequences encoding the variable regions of the
exemplary antigen binding proteins
1. Clone 1G2
Heavy Chain Variable Region (V_{H}):
CAGGTGCAACTAAAAGAGTCAGGGGGAAGCTTGGTGCAGCCTGGGAAATCTCT
AAAACTCACCTGTGTCACCTCAGGATTCACTTTCAGTACCGCCTGGATGCACTG
GGTTCGCCAGTCTCCAGAAAAACAACTCGAGTGGGTTGCTCAAATTAAAGCCA
AATCAAACAATTATGCAACCTACTATGCGGAGTCTGTTAAAGGAAGATTCACC
ATCTCAAGAGATGATTCCAAAAACAGCGTTTATTTGCAGATGAACAGCTTAAA
AGAGGAAGACACTGCCATTTATTATTGTACGGGAGGGAATAGTGTTGATTACT
GGGGCCAAGGAGTCATGGTCACAGTCTCCTCAG (SEQ ID NO: 68)
Light Chain Variable Region (V_{L}):
AACATTGTGCTGACTCAGTCTCCTTCATTCCTGCCTGCATCTGTGGGAGACAGA
GTCACTATCAACTGCAAAGCAAGTCAGAATATTAACAAGTTCTTAAACTGGTA
TCAGCAAAAGCTTGGAGAGGCCCCCAAACGCCTGATATATAATACAAACAATT
TGGACACAGGCACCCCATCAAGGTTCAGTGGCAGTGGGTCTGGTACAGATTAC
ACACTCACCATCAGCAGCCTGCAGCCTGAAGATTTTGCCACATATTTCTGCTTA
CAGCATATTCGTTTTCCCGGGACGTTCGGTGGAGGCACCATGTTGGAATTGAG
AC (SEQ ID NO: 69)
2. Clone 6D7
Heavy Chain Variable Region (V_{H}):
CAGGTACAACTAAAGGAGTCAGGGGCTGCACTGGTGAAGCCTGGAGCCTCTGT
GAAGATGTCTTGCAAGGCTTCTGGTTATACATTCACTGACCACTGGGTGAGCTG
GGTGAGACAGAGTCATGGAAGGAGCCTTGAGTGGATTGGGGAAATATCTCCTA
ACTACGATACTACTAACTTCAATGAAAAGTTCAGGGGCAGGGCCACATTGACT
GTCGACAAATCCACCAGCACAGCCTCTATGGAGCTCAGCAGATTGACATCTGA
GGACTCTGCAATCTACTACTGTTCAAGAACGAACTCAGGCTGGTACTTTGACTT
CTGGGGCCCAGGAACCATGGTCACCGTGTCTTCAG (SEQ ID NO: 70)
Light Chain Variable Region (V_{L}):
AACATTGTCATGACTCAGTCTCCAACAACCACGGCTGCATCTCCAGGGGAGAA
GGTGACCATCACCTGCCTTGCCAGCTCCAGTGTAAGGAACATATACTGGTACC
AGCAGAAGTCAGGCGCTTCCCCCAAACCCTTGATTTATTCCACATCGAGCTTGG
CTTCCGGAGTCCCAGATCGCTTCAGTGGCAGTGGGTCTGGGACCTCTTATTCTC
TCACAATCAACACCATGGAGGCTGAAGATGCTGCCACTTATTACTGTCAGCAG
TGGAGTAGGAACCCACCTACGTTCGGTTCTGGGACCAAGTTGGAGATCAAAC
(SEQ ID NO: 71)
3. Clone 9C10
Heavy Chain Variable Region (V_{H}):
CAGGTGCAGCTAAAGGAGTCAGGGGGAAGCTTGGTGCAGCCTGGGAAATCTCT
AAAACTCACCTGTGCCACCTCAGGATTCACTTTCAGTAACGCCTGGATGCACTG
GGTTCGCCAGTCTCCAGAGAAACAACTAGAGTGGGTTGCTCAAATTAAAGCCA
AATCAAACAATTTTGCAACCTACTATGCGGAGTCTGTGAGAGGAAGATTCATC
ATCTCGAGAGATGATTCCAAAAGCACCGTTTACCTGCAGATGAACAACTTAAG
AGAGGAAGACACTGCCATTTATTTCTGTGCCGGCGGTAACACCTGGGATTACT
GGGGCCAAGGAGTCATGGTCACAGTCTCCTCAG (SEQ ID NO: 72)
Light Chain Variable Region (V_{L}):
GCGGCCGCGAATTCGCCCTTGATCGGCGCGCCAGGTGGAGCTGAAGGAGACA
GAGTCACTATCAACTGCACAGCAAGTCAGAATATTAACAAGTTCTTAAACTGG
TATCAGCAAAGACTTGGAAAAGCTCCCAAACGCCTGATATTGAGTCCAAACAA
TTTGCAAGCAGGCGTCCCATCAAGGTTCAGTGGCAGTGGATCTGGTACAGATT
ACACACTCACCATCAGCAACCTGCAGCCTGAAGACTTTGCCTCATATTTCTGCT
TGCAGCATGCTAGTTTTCCCGGGACGTTCGGTGGAGGCACCAAGCTGGAATTG
ATCC (SEQ ID NO: 73)
4. Clone 26C9
Heavy Chain Variable Region (V_{H}):
CAGGTGCAGCTGAAAGAGTCAGGCCCTGGGATATTGCAGCCCTCCCAGACCCT
CAGTCTGACTTGCACTTTCTCTGGGTTTTCACTGAGCACTTATGGTGTGGGTGT
GGGCTGGATTCGTCAGCCTTCAGGGAAGGGTCTGGAGTGGCTGGCAAACATTT
GGTGGGATGATGATAAGTACTACAATCCATCTCTGAAAAACCGGCTCACAATC
TCCAAGGACACCTCCAACAATCAAGTGTTCCTCAAGATCACCAATGTGGACAC
TGCAGATACTGCCACATACTACTGTGCTCGGATAGATGGTACCCGGTACTTTGA
TTACTGGGGTCAAGGAGTCATGGTCTCAGTCTCCTCAG (SEQ ID NO: 74)
Light Chain Variable Region (V_{L}):
GACATTGTGTTGACTCAGTCTCCTCCCTCCCTGTCTGCATCTCTGGGAGACAAG
GTCACCATCACTTGCCAGACAAGTCAAAACATTAACAAGTTTATAGCTTGGTAT
CAGCAAAAGCCTGGAAAAGCTCCTAGGCAGCTCATACGTTACACTTCTACACT
GATGTCAGGTACCCCATCGAGGTTCAGTGGCAGTGGATCTGGGACAGATTATT
CATTCAGCATCAGCAACGTGGAGTCTGAAGATATTGCAACTTATTATTGTCTAC
AGTACGGTAACCTGTGGACGTTCGGTAGAGGCACCAAGCTGGAATTGAAGC
(SEQ ID NO: 75)
5. Clone 30C5
Heavy Chain Variable Region (V_{H}):
CAGGTACAACTAACGGAGTCAGGAGCTGAACTGGCAAAGCCTGGCTCTTCAGT
GAAGATTTCCTGCAAGGCTTCTGGCTACATTTTCACCAGCTACTATATAAGCTG
GATAAAACAGACGACTGGACAGGGCCTTGACTATGTTGGATATATTAATACGG
GAAATGGAGGTACTCACTACAGTGAGAGGTTCAAGGACAGCGCCACATTGACT
GTAGACAGATCCTCCAGCACAGCCTTCATGCAACTCAGCAGCCTGACACCTGA
CGACTCTGCGGTCTATTACTGTGCAAGAGGCCATCCATACTATGGATATTACCT
TGATTACTGGGGCCAAGGAGTCATGGTCACAGTCTCCTCAG (SEQ ID NO: 76)
Light Chain Variable Region (V_{L}):
GACATTGTCTTGACTCAGTCTCCTGCCTCCCTGTCTGCATCTCTGGAGGAAATT
GTCACCATCACATGCCAGGCAAGCCAGGACATCGGTAATTGGTTATCATGGTA
TCAGCAGAGCCCAGGGAAATCTCCTCAGCTCCTGATCTATGGTGCAACCAGCT
TGGCAGATGGGGTCCCATCAAGGTTCAGCGGCAGTAGATCTGGCACACAGTTT
TCTCTTACAATCAGCAGACTGCAGGTTGAAGATATTGGAATCTACTACTGTCTA
CAGACTTATACTAGTCCGTGGACGTTCGGTGGAGGCACCAAACTGGAACTGAA
AC (SEQ ID NO: 77)
6. d26C9
Heavy Chain Variable Region (V_{H}):
CAAGTGCAGCTGAAGGAGAGCGGCCCCGGCATCCTGCAGCCTTCTCAGACCCT
GAGCCTGACCTGCACCTTCAGCGGCTTCAGCCTGAGCACCTACGGCGTGGGCG
TGGGCTGGATCAGACAGCCTAGCGGCAAGGGCCTGGAGTGGCTGGCCAACATC
TGGTGGGACGACGACAAGTACTACAACCCTAGCCTGAAGAACAGACTGACGA
TTAGCAAGGACACAAGCAACAACCAAGTGTTCCTGAAGATCACCAACGTGGAC
ACCGCCGACACCGCCACCTACTACTGCGCTAGAATCGACGGCACAAGATACTT
CGACTACTGGGGCCAAGGCGTGATGGTGAGCGTGAGCAGC (SEQ ID NO: 89)
Light Chain Variable Region (V_{L}):
GACATCGTGCTGACACAGAGCCCCCCTAGCCTGAGCGCAAGCCTGGGCGACAA
GGTGACCATCACCTGTCAGACATCTCAGAACATCAACAAGTTCATCGCCTGGT
ATCAGCAGAAGCCCGGCAAGGCCCCTAGACAGCTGATCAGATACACAAGCAC
CCTGATGAGCGGCACCCCTAGCAGATTCAGCGGCAGCGGCAGCGGCACCGACT
ACAGCTTCAGCATCAGCAACGTGGAGAGCGAGGACATCGCCACCTACTACTGC
CTGCAGTACGGCAACCTGTGGACCTTCGGCAGAGGCACCAAGCTCGAGATCAA
G (SEQ ID NO: 90)

### Antibody Pairs

In some aspects, the present invention is directed to an antibody pair. In some embodiments, the antibody pair is a capture-detection pair where one of the antibodies in the pair captures sTNFR2 in a sample while the other antibody in the pair detect the sTNFR2 captured by the first antibody, allowing the quantification of sTNFR2 in the sample.

In some embodiment, the antibody pair includes a first antibody and a second antibody, which do not compete for the same epitope on sTNFR2.

In some embodiments, one of the antibodies in the antibody pair is an antibody described elsewhere herein, such as in the “Antibody” section, and the other of the antibodies in the antibody pair is not an antibody described herein, such as a commercially available STNFR2 antibody.

In some embodiments, both the first antibody and the second antibody are antibodies described elsewhere herein, such as in the “Antibody” section.

In some embodiments, the first antibody and the second antibody each independently includes:
(a) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:1, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:2, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:3; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:5, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:6;
(b) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:7, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:8, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:9; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 10, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 11, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:12;
(c) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 13, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:14, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:15; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:16, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:17;
(d) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:18, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:19, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:20; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:21, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:22;
(e) a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:23, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:24, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:25; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:26, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:27, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:28.

In some embodiments, the first antibody includes a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO: 18, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 19, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:20; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:4, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:21, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO: 22, and the second antibody includes a heavy chain polypeptide including a CDR1 including an amino acid sequence that is about 80%, 85%, 90%, 91%, 92%, 93%, 94%, 95%, 96%, 97%, 98%, 99% or 100% identical to the amino acid sequence of SEQ ID NO:7, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO: 8, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:9; and a light chain polypeptide including CDR1 including an amino acid sequence that is about 80%, 85%, 90%, 91%, 92%, 93%, 94%, 95%, 96%, 97%, 98%, 99% or 100% identical to the amino acid sequence of SEQ ID NO:10, a CDR2 including an amino acid sequence that is about 80%, 85%, 90%, 91%, 92%, 93%, 94%, 95%, 96%, 97%, 98%, 99% or 100% identical to the amino acid sequence of SEQ ID NO:11, and a CDR3 including an amino acid sequence that is about 80%, 85%, 90%, 91%, 92%, 93%, 94%, 95%, 96%, 97%, 98%, 99% or 100% identical to the amino acid sequence of SEQ ID NO:12.

In some embodiments, the first antibody and the second antibody each independently includes one of the following:
(a) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:29; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:30;
(b) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:31; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:32;
(c) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:33; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:34;
(d) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:35; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:36;
(e) a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:37; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:38.

In some embodiments, the first antibody includes a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO:35; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:36, and the second antibody includes a heavy chain polypeptide including a heavy chain variable region including an amino acid sequence that is about 80%, 85%, 90%, 91%, 92%, 93%, 94%, 95%, 96%, 97%, 98%, 99% or 100% identical to the amino acid sequence of SEQ ID NO: 31; and a light chain peptide including a light chain variable region including an amino acid sequence that is about 80%, 85%, 90%, 91%, 92%, 93%, 94%, 95%, 96%, 97%, 98%, 99% or 100% identical to the amino acid sequence of SEQ ID NO:32.

In some embodiments, the antibody pair quantifies sTNFR2 in a sample, and the first antibody captures sTNFR2 in the sample and the second antibody quantifies the sTNFR2 captured by the first antibody.

Method of Quantifying sTNFR2

In some aspects, the present invention is directed to a method of quantifying sTNFR2 in a sample.

In some embodiments, the method includes: capturing sTNFR2 in the sample with a first sTNFR2 antibody; and detecting the sTNFR2 captured by the first antibody with a second sTNFR2 antibody, thereby quantifying the captured sTNFR2.

In some embodiments, one of the first sTNFR2 antibody or the second sTNFR2 is an antibody described herein, such as but not limited to in the “Antibody” section, and the other of the first sTNFR2 antibody or the second sTNFR2 is not an antibody described elsewhere herein, such as a commercially available antibody.

In some embodiments, both the first sTNFR2 antibody and the second sTNFR2 are antibodies described herein, such as but not limited to in the “Antibody” section.

In some embodiments, the sTNFR2 in the sample is quantified using the antibody pairs described herein, such as but not limited to in the “Antibody Pair” section.

In some embodiments the method is a western blotting-based quantification method, an enzyme-linked immunoassay (ELISA)-based quantification method, or combinations thereof.

In some embodiments, sample is a serum sample, a plasma sample, a urine sample, or combinations thereof.

In some embodiments, the sample is a sample from a subject suspected to have a disease or disorder indicated by altered levels of sTNFR2, and the quantification of sTNFR2 in the sample diagnosis the disease or disorder. In some embodiments, the disease is Kawasaki disease (K_{D}) or Kawasaki-like syndrome (KLS).

In some embodiments, the sample is a sample from a subject confirmed to have a disease or disorder involving altered levels of sTNFR2, and wherein the quantification of STNFR2 predicts outcomes of the disease or disorder or monitors the state of the disease or disorder during therapies.

In some embodiments, the disease or disorder is lupus nephritis (Parodis et al., Scand J Rheumatol. 2017 July; 46:263-272), Behçet's disease (Turan et al., Scand J Rheumatol. 2008 March-April; 37:135-41), Sjogren's syndrome (Nishikawa et al. Arthritis Res Ther 2016; 18:106), rheumatoid arthritis (Cope et al.; Arthritis and rheumatism. 1992; 35:1160-9) and juvenile chronic arthritis (de Benedetti et al.; 36:581-8), inflammatory bowel diseases (Owczarek et al.; Pol Arch Med Wewn. 2012; 122:616-23) sarcoidosis (Verwoerd A et al., Clin Exp Immunol. 2016; 185:263-70), Grave's disease (Mysliwiec et al.; Immunol Invest. 2004; 33:61-8), myocardial infarction (Paccalet et al.; Front Pharmacol. 2021; 12:656928), congestive heart failure (Deswal et al.; Circulation 2001; 103:2055-9), progressive diabetic kidney disease in subjects with type 2 diabetes mellitus (Wu et al., PLOS ONE. 2022; 17:e0266854), erythropoietin resistance in setting of hemodialysis (Kato et al.; Nephrol Dial Transplant 2001; 16:1838-44), steatohepatitis (Hui et al.; Hepatology. 2004; 40:46-54), hepatitis C virus liver fibrosis (Moura et al., Eur J Clin Microbiol Infect Dis. 2010; 29:1153-61), or presence and/or prognosis of infections including sepsis (Hendrickson et al., Pulmonary Circulation. 2018; 8:1-12), malaria (Thevenon et al., J Immunol. 2010; 185:7115-22), COVID-19 (Sancho Ferrando et al.; Cytokine. 2022; 149:155727) HIV (Bilello et al.; J Infect Dis 1996; 173:464-7).

Method of Diagnosing, and/or Treating, Ameliorating, and/or Preventing Kawasaki Disease or Kawasaki-Like Syndrome

Kawasaki Disease (K_{D}) is a febrile childhood vasculitis with potentially catastrophic clinical outcomes. Untreated, one in five children develop coronary artery aneurysms causing significant morbidity and mortality.

Pediatric Kawasaki Disease (K_{D}) is diagnosed clinically based on a constellation of signs and physical exam findings including a fever ≥5 days plus at least four of the following five physical findings: non-exudative conjunctivitis, rash, changes of the oropharynx, erythema and/or painful swelling of the hands and feet, and a sentinel lymph node (>1 cm and tender). Kawasaki Disease is typically seen in children >6 months <8 years of age. Epidemiology of the disease includes periodic outbreaks suggestive of a ubiquitous infectious etiology, with an atypical presentation in a subset of individuals who come to medical attention as having Kawasaki Disease. KD inflammation has predilection for coronary arteries, with serious sequelae including development of coronary artery aneurysms and residual long-term risk for increased cardiovascular morbidity and mortality. In children, therapy with intravenous immunoglobulin (IVIG) reduces morbidity and mortality from Kawasaki disease by decreasing the proportion of patients with coronary artery aneurysm formation from 20% to 3%-4%. Diagnosis of KD using clinical criteria is problematic as children presenting with partial syndromes (fever plus 3 or fewer physical findings) may not receive IVIG and aspirin therapy but remain at risk for developing coronary artery aneurysms and sudden death.

Kawasaki Disease is extremely rare in adults. In the 1980s cases of an adult febrile syndrome that resembled pediatric Kawasaki Disease came to medical attention. The majority of the adults affected were noted to be HIV+, most with advanced disease and low CD4 counts. HIV+ individuals with Kawasaki-like syndrome (KLS), also known as Kawasaki Disease-like syndrome (KDLS), commonly experience a gastrointestinal disturbance with diarrhea and/or abdominal pain followed within several weeks by development of the protean manifestations of the syndrome including fevers, conjunctivitis, rash, changes of the oropharynx (strawberry tongue, fissures, cheilitis), painful erythema+/−swelling of the hands and feet, usually without a sentinel lymph node. Curiously, children with post-COVID-19 multisystem inflammatory syndrome (MIS-C) have the prominent GI symptoms similar to adults with HIV KLS. HIV KLS patients respond to IVIG and aspirin, and therefore are treated similarly to pediatric KD patients.

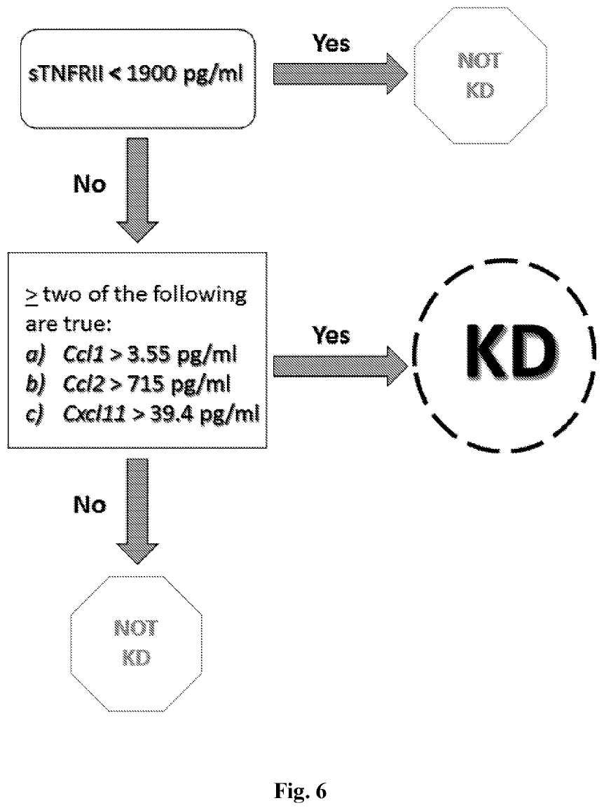

In 2016, Johnson et al (Open Forum Infect Dis 2016; 3: ofw160) published a clinical investigation of serum biomarkers in HIV positive adults with Kawasaki-like Syndrome (KLS) and a prospective clinical investigation of serum biomarkers in children with Kawasaki Disease (K_{D}), comparing them to healthy peers and peers with febrile illnesses. That investigation generated a diagnostic algorithm (see FIG. 6) that successfully separated children with KD from there febrile and healthy peers with high sensitivity and specificity. The first step in the algorithm is measurement of sTNFR2. A level of sTNFR2 below a threshold value is sufficient to exclude the possibility of Kawasaki Disease, an important consideration for whether febrile children need to be hospitalized for further evaluation and useful as a KD screening tool.

It has been shown that HIV KLS and pediatric Kawasaki Disease are likely the same malady based on a shared unique cytokine signature useful for making an objective diagnosis of Kawasaki Disease based on sTNFR2, (or 4 cytokine/chemokines including sTNFR2 as well as Ccl1, Ccl2, and Cxcl11).

Unfortunately, there is no validated CLIA-certified testing available for measuring STNFR2 levels currently available. In the study described in Johnson et al, measurement of STNFR2 was performed by Aushon Biosystems using a multiplex ELISA. The antibodies in the Aushon Biosystems kit, as well as standards used in the multiplex ELISA assay, is proprietary. Currently, virtually all major human clinical investigations measure human STNFR2 levels based on R & D Systems ELISA kits that use a goat anti-human sTNFR2 polyclonal antiserum as the detection step.

All current major clinical investigations of sTNFR2 have been and continue to be done with sTNFR2 ELISA reagents developed by R & D systems that include a polyclonal goat anti-human sTNFR2 detection reagent. As described elsewhere herein, the instant antibodies have both higher sensitivity and higher specificity (e.g., does not cross-react with sTNFR1) when comparing to the polyclonal goat antibodies.

Accordingly, in some aspects, the present invention is directed to a method of diagnosing a Kawasaki disease (K_{D}) or Kawasaki-like syndrome (KLS).

Accordingly, in some aspects, the present invention is directed to a method of treating, ameliorating, and/or preventing a Kawasaki disease (K_{D}) or Kawasaki-like syndrome (KLS).

In some embodiments, the method includes quantifying sTNFR2 level in a sample from the subject, including capturing sTNFR in the sample with a first sTNFR2 antibody, and quantifying the sTNFR2 captured by the first sTNFR2 antibody with a second sTNFR2 antibody. In some embodiments, that the sTNFR2 level quantified by the first antibody and the second antibody is equal to or higher than a first predetermined level indicates that the subject has KD or KLS.

In some embodiments, both the first sTNFR2 antibody and the second sTNFR2 antibody are the same as or similar to the antibodies described herein, such as but not to limited to in the “Antibody” section. In some embodiments, the first sTNFR2 antibody and the second sTNFR2 antibody recognize different epitopes of sTNFR2. In some embodiments, the first sTNFR2 antibody and the second sTNFR2 antibody are the same as or similar to those described in the “Antibody Pair” section herein.

In some embodiments, only one of the first sTNFR2 antibody or the second sTNFR2 antibody is the same as or similar to the antibodies described herein, such as but not to limited to in the “Antibody” section. The other one of the first sTNFR2 antibody or the second sTNFR2 antibody is a commercially available antibody that do not substantially compete with the epitope of the antibody herein.

In some embodiments, the subject is determined, by the sTNFR2 levels, as having KD or KLS and the method further includes administering to the subject an effective amount of a medication for treating KD and KLS. Examples of medication for treating KD and KLS include intravenous immunoglobulin (IVIG), aspirin, steroids, infliximab (humanized mouse anti-TNF monoclonal antibody, sold under the commercial name Remicade), or the like. In some embodiments, the medication for treating KD and KLS includes one or more of the antibodies herein. The treatment of KD and KLS sometimes require the administration of infliximab, which targets TNF and blocks the binding of TNF to TNFR2/sTNFR2. Due to the existence of residual mouse epitope in the infliximab (i.e., incomplete humanization of the mouse antibody), patients' bodies sometimes have immune response to infliximab and cause the treatment to fail. The antibodies herein, especially those generated from humanizing antibodies of rat origins, provide an alternative strategy for blocking the binding of TNF to TNFR2/sTNFR2.

In some embodiments, sample is a serum sample, a plasma sample, a urine sample, or combinations thereof.

In some embodiments, the method further includes: quantifying C—C motif chemokine ligand 1 (Ccl1) level in the sample; quantifying C—C motif chemokine ligand 2 (Ccl2) level in the sample; and quantifying C—X—C motif chemokine 11 (Cxc11) level in the sample.

In some embodiments, the subject is confirmed to have KD or KLS if the sTNF2 level in the sample is equal to or higher than the first predetermined level and at least two of the following are true:
(a) the quantified Ccl1 level is higher than a second predetermined level,
(b) the quantified Ccl2 level is higher than a third predetermined level,
(c) the quantified Cxcl11 level is higher than a fourth predetermined level

Methods of quantifying Ccl1, Ccl2 and Cxcl11 are known in the art and products for quantifying these proteins are commercially available.

Methods for diagnosing and treating KD and KLS is described in detail in US Patent Application Publication No. 2019/0302113 A1, the entirety of this reference is hereby incorporated by reference.

Methods of Diagnosing, and/or Treating, Ameliorating, and/or Preventing Inflammatory Diseases or Syndromes Mediated by TNFα-sTNFR2 Signaling

TNFα interacts with both TNFR1 and TNER2 to mediate downstream signalings. The two receptors of TNFα have different expressions as well as functions. For example, TNFR1 is ubiquitously expressed on nearly all cells, while TNFR2 is restricted to T lymphocytes and some other types of cells (Yang et al., Cell Death & Disease volume 10, Article number: 27 (2019)). It has been indicated that while TNFR1 predominantly promotes inflammatory signaling pathways, TNFR2 mediates immune modulatory functions and promotes tissue homeostasis and regeneration (Fischer et al., Antibodies 2015, 4(1), 48-70). As such, it is expected that the specific inhibition of TNFR1 or TNFR2, rather than the inhibition of TNFα which affects both signaling, would be able provide more specific and effective treatment to various diseases or disorders mediated by TNFR1 or TNFR2.

Currently, only pan TNFα blockade is currently approved for treating such conditions. Specifically, TNFα blockade have been approved for treating inflammatory conditions such as rheumatoid arthritis, plaque psoriasis, psoriatic arthritis, ankylosing spondylitis, Crohn's disease, ulcerative colitis, hidradenitis, uveitis, and Kawasaki disease.

Diseases with elevated levels of sTNFR2 include the following: Lupus nephritis (Parodis et al., Scand J Rheumatol. 2017 July; 46:263-272), Behçet's disease (Turan et al., Scand J Rheumatol. 2008 March-April; 37:135-41), Sjogren's syndrome (Nishikawa et al. Arthritis Res Ther 2016; 18:106), rheumatoid arthritis (Cope et al.; Arthritis and rheumatism. 1992; 35:1160-9) and juvenile chronic arthritis (de Benedetti et al.; 36:581-8), inflammatory bowel diseases (Owczarek et al.; Pol Arch Med Wewn. 2012; 122:616-23) sarcoidosis (Verwoerd A et al., Clin Exp Immunol. 2016; 185:263-70), Grave's disease (Mysliwiec et al.; Immunol Invest. 2004; 33:61-8), myocardial infarction (Paccalet et al.; Front Pharmacol. 2021; 12:656928), congestive heart failure (Deswal et al.; Circulation 2001; 103:2055-9), progressive diabetic kidney disease in subjects with type 2 diabetes mellitus (Wu et al., PLOS ONE. 2022; 17: e0266854), erythropoietin resistance in setting of hemodialysis (Kato et al.; Nephrol Dial Transplant 2001; 16:1838-44), steatohepatitis (Hui et al.; Hepatology. 2004; 40:46-54), hepatitis C virus liver fibrosis (Moura et al., Eur J Clin Microbiol Infect Dis. 2010; 29:1153-61), or presence and/or prognosis of infections including sepsis (Hendrickson et al., Pulmonary Circulation. 2018; 8:1-12), malaria (Thévenon et al., J Immunol. 2010; 185:7115-22), COVID-19 (Sancho Ferrando et al.; Cytokine. 2022; 149:155727) HIV (Bilello et al.; J Infect Dis 1996; 173:464-7).

While pan TNFα blockade has positively impacted human health, there have been some notable failures including sepsis (clinicaltrials.gov NCT01145560) and asthma. The published asthma clinical trial with a pan TNFα blocker was stopped early for increased incidence of severe infections and malignancy without major improvement in pulmonary functions. Clinical response rates to pan TNFα blockade range from roughly 20%-60% depending on disease and clinical response criteria applied. It is unclear whether pan TNFα or selective TNFR1 antagonism represents optimal interventions in inflammatory diseases, and therefore there is a need for investigations with TNFR2 selective agents to address safety and efficacy issues with existing therapies.

As detailed elsewhere herein, the present study identified antibodies that are able to specifically bind to TNFR2 extracellular domain and block or reduce the interaction between TNFR2 and TNFα. It is expected that the blockade/reduction of this interaction is able to treat, ameliorate and/or prevent diseases or disorders mediated by hyperactive TNFα-TNFR2 signaling.

In one aspect, the disclosure provides a method of monitoring progression and/or state of a disease or disorder involving altered levels of sTNFR2 in a subject diagnosed with the disease or disorder.

In certain embodiments, the method comprises obtaining subject's samples at two or more time points after diagnosis of the disease or disorder in the subject.

In certain embodiments, the method comprises capturing sTNFR2 in each obtained sample with the first antibody of the antibody pairs described herein.

In certain embodiments, the method comprises quantifying sTNFR2 captured by the first antibody with the second antibody of the antibody pairs described herein.

In certain embodiments, the method comprises comparing the sTNFR2 levels quantified by the first antibody and the second antibody in the obtained subject's samples.

In certain embodiments, the comparing step allows for monitoring progression and/or state of the disease or disorder in the subject.

In certain embodiments, at least one of the two or more time points is a time point where the subject is receiving therapeutic treatment for the disease or disorder.

In certain embodiments, the comparing step allows for monitoring therapeutic outcome of the therapeutic treatment in the subject.

In certain embodiments, the method of quantifying is a western blotting-based method, an enzyme-linked immunoassay (ELISA)-based method, or combinations thereof.

In certain embodiments, each sample is independently a serum sample, a plasma sample, a urine sample, or combinations thereof.

In certain embodiments, the disease or disorder is lupus nephritis, Behçet's disease, Sjogren's syndrome, rheumatoid arthritis and juvenile chronic arthritis, inflammatory bowel diseases, sarcoidosis, Grave's disease, myocardial infarction, congestive heart failure, progressive diabetic kidney disease in subjects with type 2 diabetes mellitus, erythropoietin resistance in setting of hemodialysis, steatohepatitis, hepatitis C virus liver fibrosis, post-COVID-19 multisystem inflammatory syndrome (MIS-C) in children, presence and/or prognosis of infections including sepsis, malaria, COVID-19, or HIV.

Accordingly, in some embodiments, the present invention is directed to a method of treating, ameliorating and/or preventing a disease or disorder caused by or involves overactive TNFα-TNFR2 signaling in a subject in need thereof.

In some embodiments, the disease or disorder includes overexpression of IFN-γ.

In some embodiments, the method includes administering to the subject an effective amount of an antibody that bind to TNFR2 and blocks/reduces TNFα-TNFR2 interaction. In some embodiments, the antibody is the same as or similar to those as described elsewhere herein, such as in the “Antibodies” section.

In some embodiments, the disease or disorder includes at least one selected from the group consisting of lupus nephritis, plaque psoriasis, psoriatic arthritis, ankylosing spondylitis, Crohn's disease, ulcerative colitis, hidradenitis, uveitis, Kawasaki disease, Behçet's disease, Sjogren's syndrome, rheumatoid arthritis and juvenile chronic arthritis, juvenile rheumatoid arthritis with macrophage activation syndrome, inflammatory bowel diseases, sarcoidosis, Grave's disease, myocardial infarction, congestive heart failure, progressive diabetic kidney disease in subjects with type 2 diabetes mellitus, steatohepatitis, hepatitis C virus liver fibrosis, post-COVID-19 multisystem inflammatory syndrome (MIS-C) in children and adults (MIS-A).

In some embodiments the disease or disorder includes infections with TNFα immunopathology where TNFα blockade was ineffective, including bacterial sepsis, detrimental or ineffective in human diseases including asthma, interstitial lung disease and cerebral malaria, or uninvestigated due to presumed risk in setting of infections including ARDS and diffuse alveolar hemorrhage (DAH) associated with viral infections including COVID-19, and bacterial infections including leptospirosis).

In some embodiments, the method reduces a level of IFN-γ in the body of the subject. In some embodiments, the method reduces the expression of IFN-γ by PBMC cells.

In some embodiments, the antibody herein is administered as part of a composition. In some embodiments, the composition herein includes at least one antibody herein, and at least one pharmaceutically acceptable carrier. The formulation of the antibody herein for administration, as well as the route of administration, are described elsewhere herein, such as in the “Administration/Dosage/Formulations” section.

In some embodiments, the subject is a mammal, such as a human.

### Combination Therapies

In some embodiments, the method of treating, ameliorating, and/or preventing the disease/disorder includes administering to the subject the effective amount of at least one compound and/or composition contemplated within the disclosure.

In some embodiments, the composition for treating the disease/disorder herein includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating the disease/disorder, and/or at least one additional agent that treats one or more diseases or disorders contemplated herein.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject prior to, during, or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compounds of the disclosure are formulated as a composition with one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of the disease/disorder herein in a patient.

Formulations may be employed in admixtures with conventional excipients, i.e., pharmaceutically acceptable organic or inorganic carrier substances suitable for intracranially, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. patents applications 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of disease/disorder in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED50 with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Generation and Characterization of Anti-sTNFR2 Antibodies

Two fusion proteins with the extracellular domain of TNFRII linked to the Fc domain of rat IgG2b, termed sTNFR2sig 1 and sTNFR2sig 2, were expressed in cultured 293F cells and purified from the culture supernatant.

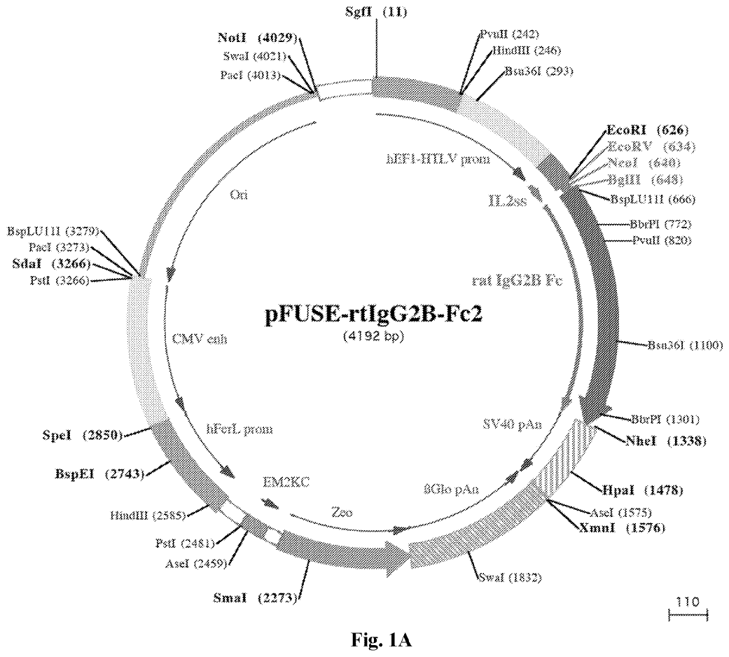

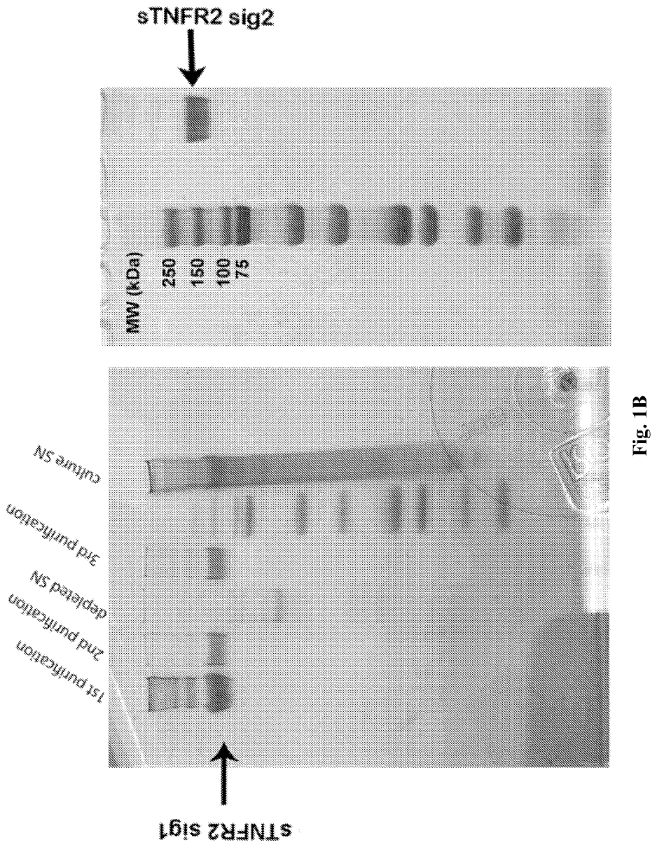

Specifically, for sTNFR2sig 1, TNFR2 extra cellular domain were amplified with the following primers: 5′ GATATCTGTGTTGCCCGCCCAGGTGGCATTTAC 3′ (human TNFR2 extracellular domain forward primer with EcoRV restriction endonuclease recognition sequence, SEQ ID NO:78) and 5′ GATATCGCGTCGCCAGTGCTCCCTTCAGCTG 3′ (human TNFR2 extracellular domain reverse primer with EcoRV restriction endonuclease recognition sequence, SEQ ID NO:79). The amplification product was digested with EcoRV and ligated into pFUSE-rtIgG2B-Fc2 plasmid (Invitrogen) (FIG. 1A) to produce the plasmid termed pFuse-ratIgG2b-Fc2-STNFR2sig1. The pFuse-ratIgG2b-Fc2-sTNFR2sig1 plasmid was introduced into 293F cells to produce the sTNFR2sig1 fusion protein, which has the following sequence:

TNFR2 extra cellular domain-rat IgG2b Fc domain fusion protein sTNFR2sig1

MYRMQLLSCIALSLALVTNSISVLPAQVAFTPYAPEPGSTCRLREYYDQTAQMCCS
KCSPGQHAKVFCTKTSDTVCDSCEDSTYTQLWNWVPECLSCGSRCSSDQVETQAC
TREQNRICTCRPGWYCALSKQEGCRLCAPLRKCRPGFGVARPGTETSDVVCKPCA
PGTFSNTTSSTDICRPHQICNVVAIPGNASMDAVCTSTSPTRSMAPGAVHLPQPVST
RSQHTQPTPEPSTAPSTSFLLPMGPSPPAEGSTGDAISAMVRSPTCPTCHKCPVPELL
GGPSVFIFPPKPKDILLISQNAKVTCVVVDVSEEEPDVQFSWFVNNVEVHTAQTQP
REEQYNSTFRVVSALPIQHQDWMSGKEFKCKVNNKALPSPIEKTISKPKGLVRKPQ
VYVMGPPTEQLTEQTVSLTCLTSGFLPNDIGVEWTSNGHIEKNYKNTEPVMDSDG
SFFMYSKLNVERSRWDSRAPFVCSVVHEGLHNHHVEKSISRPPGK (SEQ ID NO: 80)
Notes:
MYR to TNS: Signal peptide
ISV: N-terminal stabilization cap
LPA to TGD: TNFR2 extracellular domain
AIS to VRS: linker sequence
First PTC (the amino acid sequence right after the linker sequnce) to PGK: Rat IgG2b Fc

For sTNFR2sig 2, the signal peptide and the N-terminal stabilization cap portion in pFuse-ratIgG2b-Fc2-sTNFR2sig1 plasmid were replaced to result in pFuse-ratIgG2b-Fc2-STNFR2sig2 plasmid. The plasmid was introduced in to 293F cells to produce the STNFR2sig1 fusion protein, which has the following sequence:

TNFR2 extra cellular domain-rat IgG2b Fc domain fusion protein sTNFR2sig2

MAPVAVWAALAVGLELWAAAHAVLPAQVAFTPYAPEPGSTCRLREYYDQTAQM
CCSKCSPGQHAKVFCTKTSDTVCDSCEDSTYTQLWNWVPECLSCGSRCSSDQVET
QACTREQNRICTCRPGWYCALSKQEGCRLCAPLRKCRPGFGVARPGTETSDVVCK
PCAPGTFSNTTSSTDICRPHQICNVVAIPGNASMDAVCTSTSPTRSMAPGAVHLPQP
VSTRSQHTQPTPEPSTAPSTSFLLPMGPSPPAEGSTGDAISAMVRSPTCPTCHKCPVP
ELLGGPSVFIFPPKPKDILLISQNAKVTCVVVDVSEEEPDVQFSWFVNNVEVHTAQT
QPREEQYNSTFRVVSALPIQHQDWMSGKEFKCKVNNKALPSPIEKTISKPKGLVRK
PQVYVMGPPTEQLTEQTVSLTCLTSGFLPNDIGVEWTSNGHIEKNYKNTEPVMDSD
GSFFMYSKLNVERSRWDSRAPFVCSVVHEGLHNHHVEKSISRPPGK (SEQ ID
NO: 81)
Notes:
First MAP (first three amino acid residues) to AHA: Signal peptide
V after the signal peptide: N-terminal stabilization cap
LPA to TGD: TNFR2 extracellular domain
AIS to VRS: linker sequence
First PTC (the PTC right after the linker sequence) to PGK: Rat IgG2b Fc

Referring to FIG. 1B, both sTNFR2sig1 and sTNFR2sig2 fusion proteins were purified and shipped to Green Mountain Antibodies, Inc. to immunize rats and generate monoclonal antibodies.

The rat monoclonal antibodies generated were screening in a solid-phase ELISA (in which TNFR2 was coated at ~1 μg/ml) and a sandwich ELISA assay (in which mouse TNF RII/TNFRSF1B Antibody MAB726 purchased from R & D systems was coated at ~1 μg/ml as the capture antibody; 1 ng/well TNFRII was then loaded).

Among the generated monoclonal rat anti-human antibodies, five (5) antibodies were selected. The five antibodies are termed sTNFR2-1G2, sTNFR2-6D7, sTNFR2-9C10, STNFR2-26C9 and sTNFR2-30C5. Referring to Table 1, all the selected antibodies gave strong signals in the solid-phase ELISA (the “vs. TNFR2-screen” column) and the sandwich ELISA assay (the “Sandwich ELISA” column) except sTNFR2-26C9 which gave somewhat weaker signals, and do not have non-specific cross-reaction with BSA or rat IgG Fc. The sandwich ELISA screen was included to identify monoclonal antibodies that could be used as detection antibodies partnered with commercially available MAB726. Values in the table are optical density (O.D.) at 450 nm in the colorimetric assay.

TABLE 1

| | | | | vs. rat | Sandwich |
|---|---|---|---|---|---|
| Line | Clone | vs. TNFR2-screen | vs. BSA | IgG Fc | ELISA |
| 28 | 06D07 | 2.200 | 0.062 | 0.094 | 1.103 |
| 78 | 26C09 | 2.339 | 0.045 | 0.047 | 0.205 |
| 88 | 30C05 | 2.405 | 0.042 | 0.045 | 1.264 |
| 3 | 01G02 | 2.302 | 0.048 | 0.053 | 1.406 |
| 41 | 09C10 | 2.364 | 0.065 | 0.072 | 1.472 |

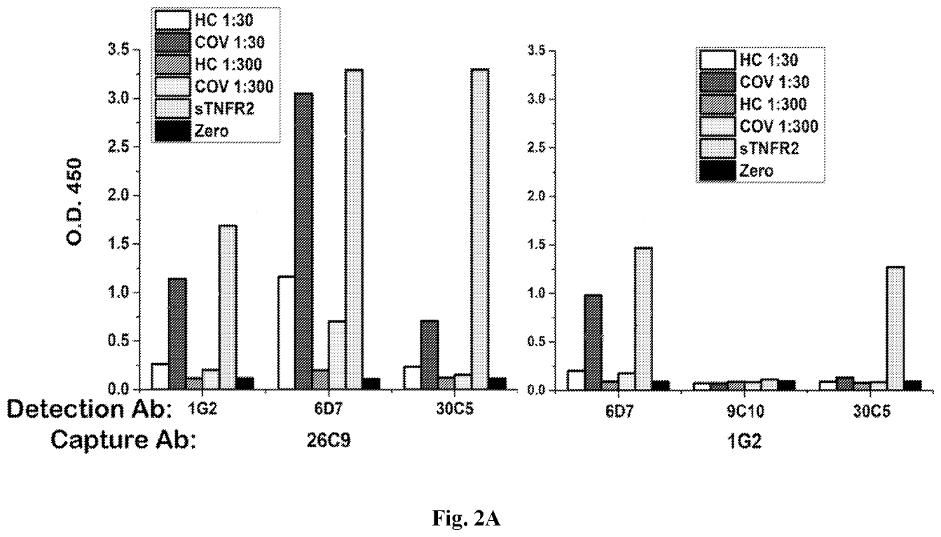

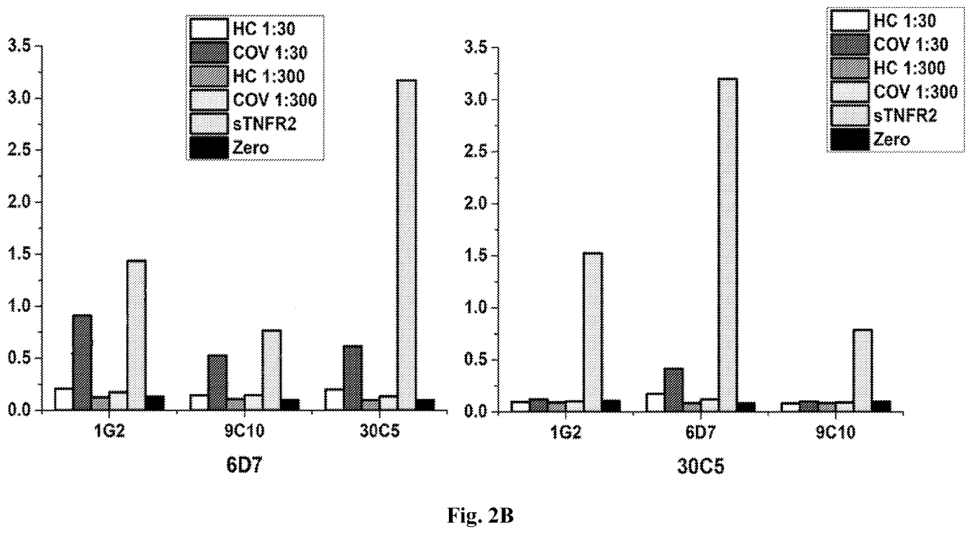

Referring to FIG. 2, these antibodies do not compete with each other for binding to the STNFR2 fusion protein and are able capture sTNFR2 from human serum. All the five antibodies were confirmed to be able to capture and/or detect sTNF2. With the exception of 1G2 and 9C10 which compete for the same epitope on sTNFR2, many pairs of the antibodies were confirmed to be useful as a capture/detect pair in ELISA-type testing.

### Example 2: Antibodies of Present Invention Detect sTNFR2 with High Sensitivities

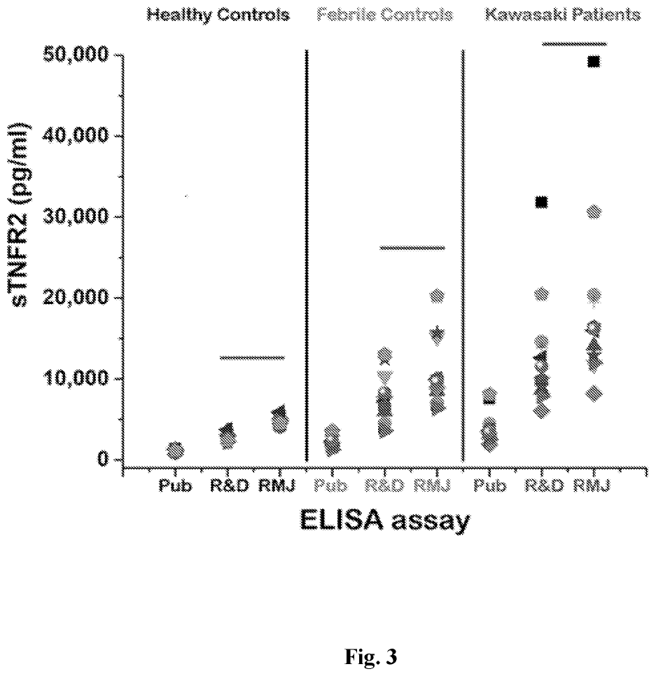

Referring to FIG. 3, sTNFR2 levels in the serum samples described in Johnson et al. (Open Forum Infect Dis 2016; 3: ofw160) were re-determined using the R & D systems STNFR2 duoset (polyclonal detection antibody) (“R & D”), and a pair of two non-limiting examples of the instant monoclonal antibodies (26C9 as capture antibody and 6D7 as detection antibody) (“RMJ”) using sandwich ELISA.

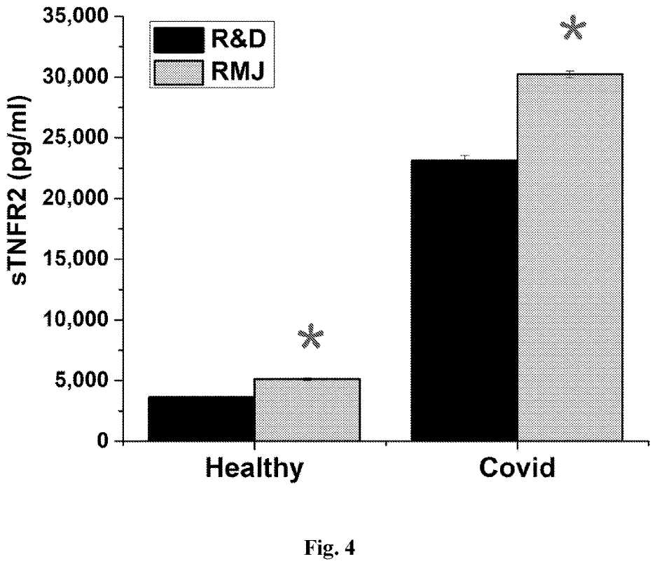

Referring to FIG. 4, sTNFR2 levels in plasma samples from a healthy control subject (“Healthy”) and a COVID-19 patient (The COVID-19 patient donated blood samples on day 4 of hospitalization, was transferred to the intensive care unit on day 10 and died on day 12 of hospitalization) (“Covid”) were determined using the R & D systems duoset (“R & D”) and the pair two non-limiting examples of the instant monoclonal antibodies (26C9 as capture antibody and 6D7 as detection antibody) (“RMJ”) using sandwich ELISA.

The results demonstrate that sandwich ELISA assays using the instant non-limiting examples of antibodies can detect more sTNFR2 in virtually all the samples. As such, the instant antibodies are significantly more sensitive in comparison to the currently available polyclonal goat antibodies used in the R & D systems product.

### Example 3: Antibodies of Present Invention do not Cross-React with sTNFR1

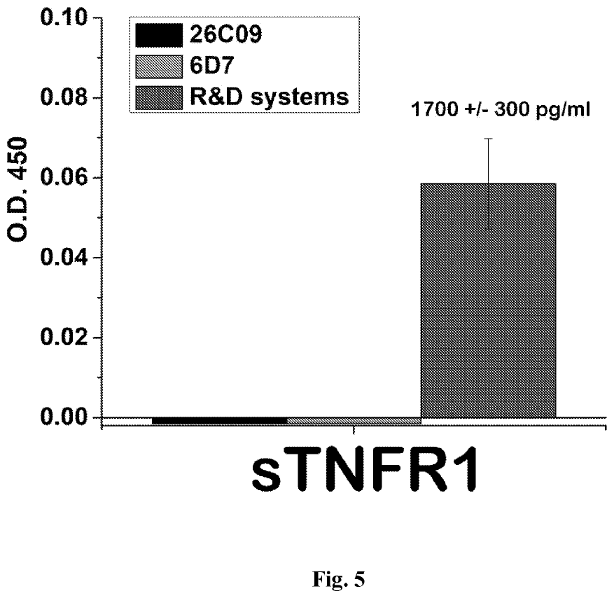

Referring to FIG. 5, sTNFR1 levels in the COVID patient plasma were measured with R & D systems Human TNF RI/TNFRSF1A DuoSet ELISA cat #DY225, or using the instant antibodies by separately substituting 26C09 for the R & D systems capture antibody and 6D7 biotinylated for the detection polyclonal antibody. Neither the sTNFR2 capture antibody 26C09 nor detection antibody 6D7 have detectable cross-reactivity with sTNFR1. The plasma of the patient with severe COVID-19 had a sTNFR1 level of 1700±300 μg/ml as determined by the R & D Systems Human TNF RI/TNFRSF1A DuoSet ELISA.

### Example 4: Static Assay for TNFα Binding and Dynamic PBMC Assay for Co-Stimulation

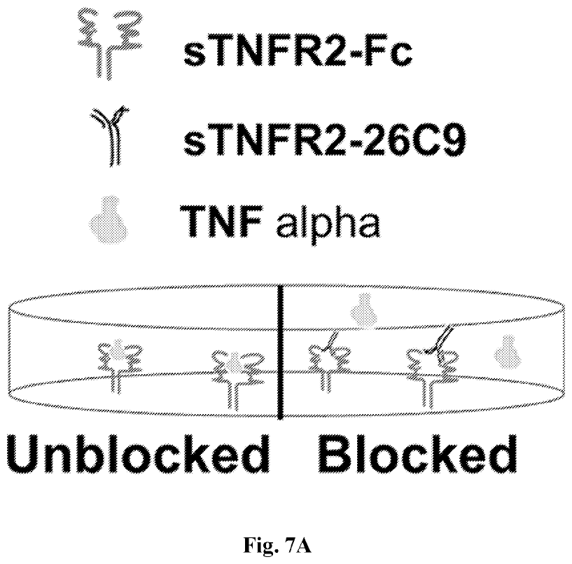

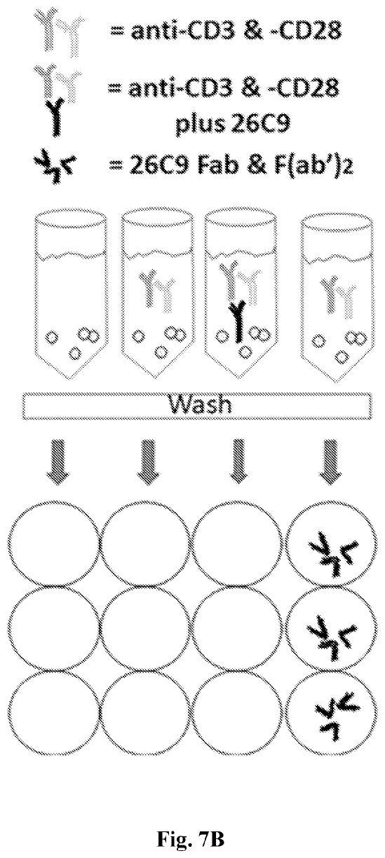

Referring to FIGS. 7A-7B, assays were developed to measure the binding of human TNFα to TNFR2 and biologic effect of TNFα/TNFR2 on costimulation (e.g., T-cell activation) using PBMC.

The present study developed an in vitro static assay for measuring the binding between TNFα to the TNFR2-rat Fc fusion protein sTNFR2sig1. Purified sTNFR2sig1 in PBS at a concentration of 2 μg/ml is applied at 50 μl/well into Nunc Maxisorp 96-well plates and incubated overnight at 4 °C. The plate is washed twice with 100 μl of 0.75×PBS with 0.01% Tween 20 (wash buffer), then blocked with PBS/0.05% Tw20/1.5% BSA (blocking buffer) for 60 minutes. Plate was washed twice with 100 μl of wash buffer, then 50 μl of blocking buffer added to all wells. For inhibition studies antibodies are added to each well at desired concentration in 25 μl of blocking buffer and incubated at room temp for 30 minutes, then recombinant human TNFα (R & D systems cat #210-TA) added in desired concentration to bring final volume to 150 μl, without removal of antibodies during inhibition studies.

After 30 minutes the plate was washed twice with 100 μl of wash buffer, then bound TNFα detected with biotinylated polyclonal goat antihuman TNFα reagent at 250 ng/ml (R & D systems cat #BAF210), 100 μl per well, in blocking buffer, incubated for 30 minutes. After 30 minutes the plate was washed twice with 100 μl of wash buffer, then bound biotinylated antihuman TNFα is detected with streptavidin-HRP (0.667 μl/ml, BD biosciences cat #554066) in blocking buffer, 100 μl per well, incubated for 20 minutes at room temperature, prewashed by shaking off streptavidin, misting plate with wash buffer (spray bottle) adding 100 μl of wash buffer then blotting the plate surface, then washing sequentially with 100 μl, 100 μl, 250 μl wash buffer and developing with 100 μl TMB reagent (Sigma cat #T4444). Assay stopped prior to saturation or 5 minutes, whichever comes first, with 50 μl of 0.32 M H₂SO₄ and read at 450 nm on an ELISA plate reader within 30 minutes.

The present study further developed a dynamic in vitro costimulation (e.g., T-cell activation) assay based on the human peripheral blood mononuclear cells (PBMCs). Consented human subjects donate blood collected in K2EDTA tubes. The blood is processed to PBMC using Lymphoprep™ tubes according to manufacturer's specifications (Abbot Diagnostics Technologies cat #1019818), then washed twice with RPMI 1640 medium supplemented with 25 mM Hepes, 5×10⁻⁵ 2 mercaptoethanol, 1× glutamax, 12.5 μg/ml gentamicin, 10% characterized fetal bovine serum (RPMI CM). Resulting PBMC are counted then resuspended at 7.5×10⁶ cells/ml in RPMI CM. According to experimental design requirements, PBMC are aliquoted into appropriately sized tubes determined by volume sufficient to number of wells needed. Control tubes do not receive antibodies. Costimulation tubes receive sufficient low endotoxin purified murine monoclonal antibodies against human CD3 (UCHT1, IgG1) CD28 (CD28.2) (Biolegend) to bring concentrations to 200 ng/ml. For transient inhibition experiments, inhibition tubes in addition to anti-CD3 &-CD28 receive sufficient azide-free experimental monoclonal antibody to bring concentration to 1.5 μg/ml.

All tubes are incubated at 4 °C for 30 minutes, then cells pelleted a 400 g×5 minutes, media aspirated, resuspended in 10 ml of RPMI CM, pelleted at 400 g×5 minutes, media aspirated then resuspended in their original volume with RPMI CM. 100 μl of cell suspensions are added to wells containing 50 μl 300 units/ml recombinant human IL-2 (Chiron) and 20 ng/ml recombinant human IL-7 (R & D systems), final concentration 75 units and 5 ng/ml respectively. For experiments with continuous inhibitor exposure, indicated inhibitor (e.g., Fab/F(ab′)2 fragments) at desired concentration are added to wells in 50 μl RPMI CM. Finally, 100 μl of experimental PBMC suspensions are added to specified wells (750k cells per well) and incubated in a humidified 5% CO₂ incubator. On day 3 supernatants are harvested from all wells for quantitation of IFN-γ that serves as a biologic readout for T cell activation. Features of the assay include transient TCR activation as exposure to anti-CD3 and -28 antibodies is limited to 30 minutes at 4 °C. Cells recovered on day 3 have usual stainable CD3 on cell surface. Use of stimulatory and costimulatory mouse antibodies for TCR activation is restricted to the IgG1 subclass that binds human Fc hFcγRIIa/b receptors on antigen presenting cells, B cells and monocytes, present in PBMC.

### Example 5: Antibodies of the Present Invention Reduced/Blocked TNFα-TNFR2 Interaction and Reduced IFN-γ Production

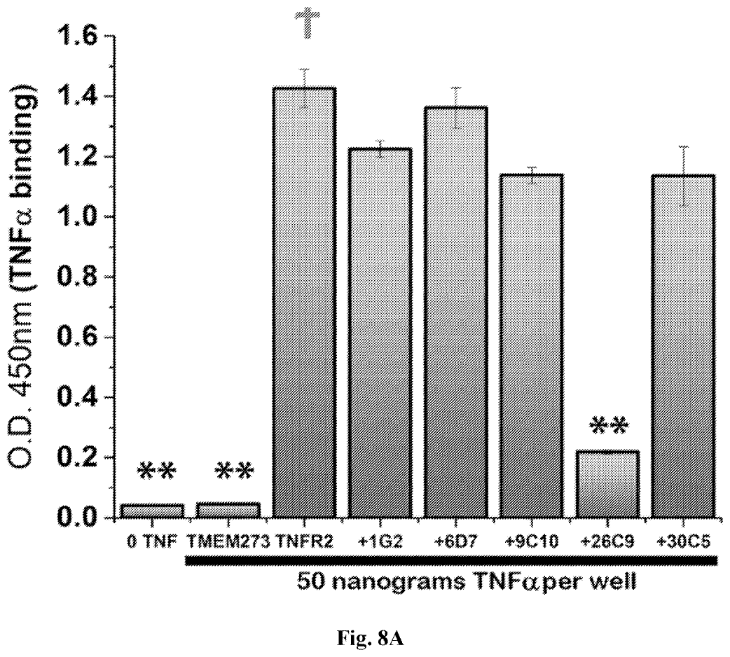

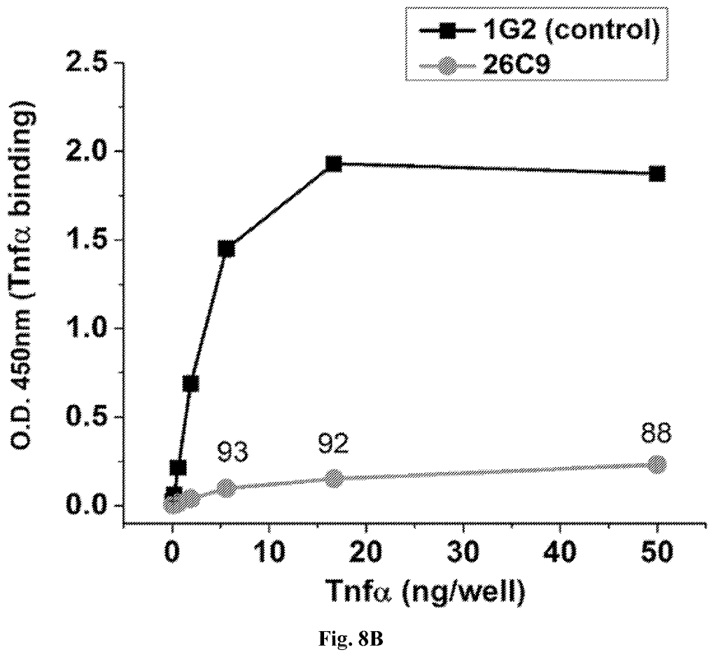

Referring to FIGS. 8A-8B, binding of recombinant human TNFα to the TNFR2 fusion protein designated sTNFR2sig1 (static assay), and screening of a panel of anti-TNFR2 antibodies for their ability to block TNF binding using the static assay identified TNFR2-26C9 as a blocking antibody.

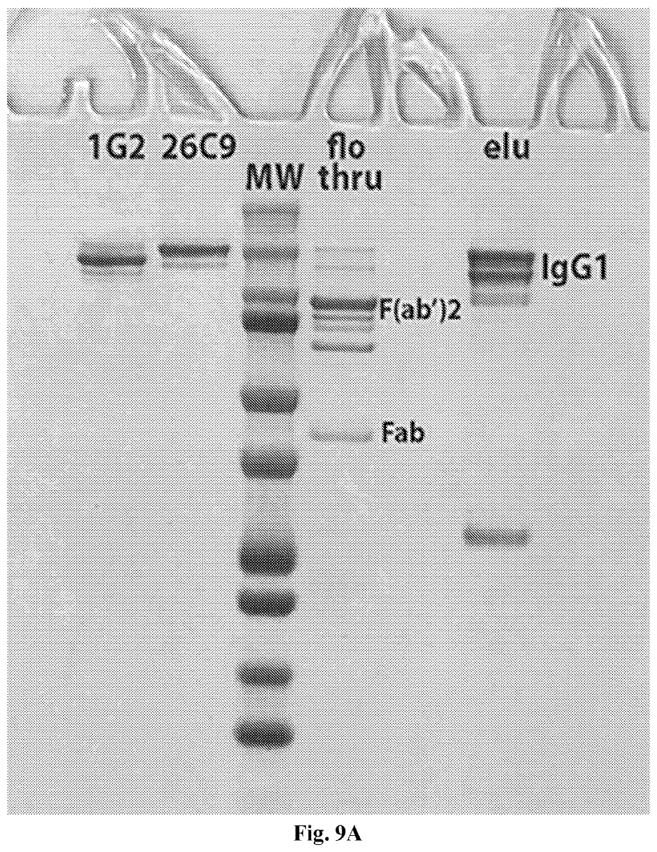

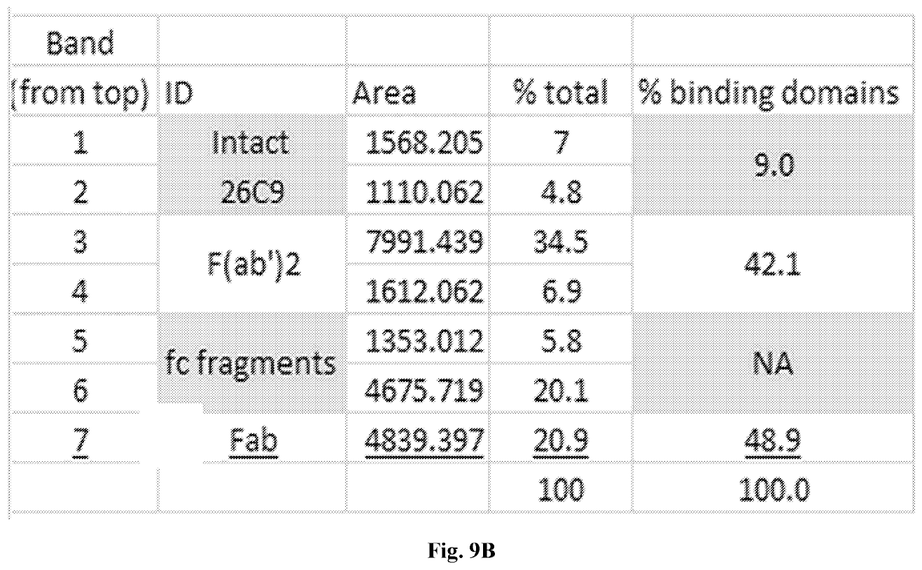

Referring to FIGS. 9A-9B, the figures depict the fragmentation of 26C9 into Fab and F(ab′)2 and relative concentration of each using mouse IgG1 Fragmentation Kit (Pierce catalog #44980).

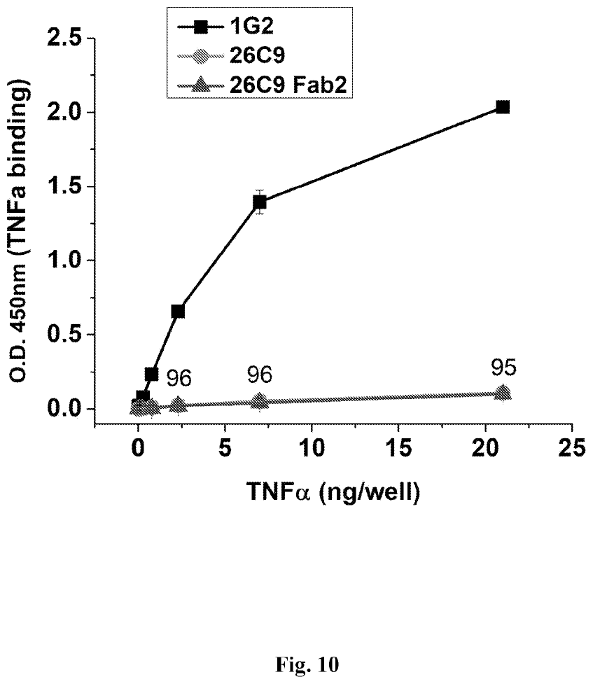

Referring to FIG. 10, the relative ability of 26C9 and 26C9 fragments to block TNFα binding to sTNFR2sig1 in the static assay was demonstrated.

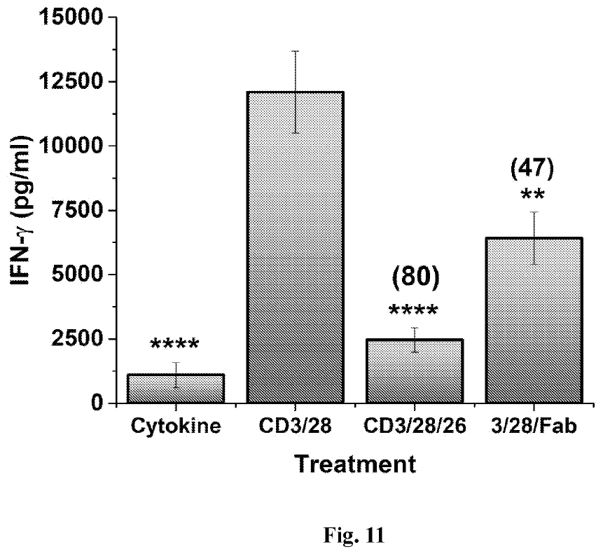

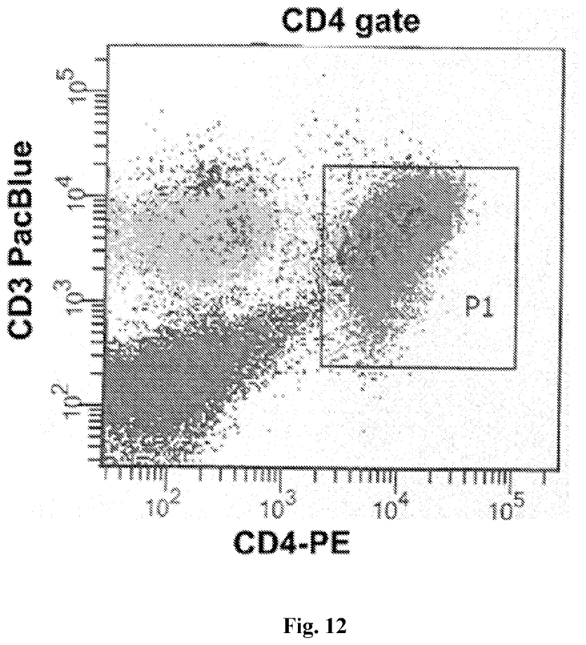

Referring to FIG. 11, the relative ability of sTNFR2-26C9 and 26C9 fragments to block PBMC IFN-γ production triggered by CD3 & CD28 in the dynamic assay was demonstrated.

### Example 6: Humanized Versions of the Antibodies of the Present Invention

In some embodiments, the antibodies of the present invention are humanized to reduce the immunogenicity of these antibodies in human subjects, which renders the antibodies more suitable for administering to a human subject, such as treating a human disease or condition with pathology driven in toto or in part by binding of TNFα to TNFR2 (such as sTNFR2 or transmembrane TNFR2).

As a non-limiting example, the present study humanized the antibody 26C9 by changing the sequence thereof, which was derived from a rat monoclonal source, into a human IgG4 recombinant monoclonal sequence. Specifically, Prometheus™ antibody humanization platform by Absolute Antibody LTD was employed to humanize the light and heavy chain backbones surrounding the 26C9 complement determining regions (CDR). The antibody humanization platform humanized the rat-derived 26C9 antibody into a human IgG4 subclass. This non-limiting humanized antibody is designated herein as h26C9.

The non-limiting humanized antibody h26C9 has the following amino acid sequences:

h26C9 heavy chain amino acid sequence:
QVQLKESGPGILQPSQTLSLTCTFSGFSLSTYGVGVGWIRQPSGKGLEWLANIWWD
DDKYYNPSLKNRLTISKDTSNNQVFLKITNVDTADTATYYCARIDGTRYFDYWGQ
GVMVSVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYGP
PCPSCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLG
K (SEQ ID NO: 82) (Note: the actual amino acid residues at the high-lighted
locations were inferred from the wildtype human IgG4 sequences rather than
determined by sequencing)
h26C9 kappa light chain amino acid sequence:
DIVLTQSPPSLSASLGDKVTITCQTSQNINKFIAWYQQKPGKAPRQLIRYTSTLMSG
TPSRFSGSGSGTDYSFSISNVESEDIATYYCLQYGNLWTFGRGTKLEIKRTVAAPSV
FIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC (SEQ ID NO: 83)

The kappa light chain DNA sequence of h26C9 is set forth below (the DNA sequence of the heavy chain was not purchase/released by Absolute Antibody, LTD, but is available):

h26C9 kappa light chain DNA sequence:

GACATCGTGCTGACACAGAGCCCCCCTAGCCTGAGCGCAAGCCTGGGCG
ACAAGGTGACCATCACCTGTCAGACATCTCAGAACATCAACAAGTTCAT
CGCCTGGTATCAGCAGAAGCCCGGCAAGGCCCCTAGACAGCTGATCAGA
TACACAAGCACCCTGATGAGCGGCACCCCTAGCAGATTCAGCGGCAGCG
GCAGCGGCACCGACTACAGCTTCAGCATCAGCAACGTGGAGAGCGAGGA
CATCGCCACCTACTACTGCCTGCAGTACGGCAACCTGTGGACCTTCGGC
AGAGGCACCAAGCTCGAGATCAAGAGAACTGTGGCCGCGCCGTCAGTGT
TTATCTTCCCTCCATCGGATGAACAGCTTAAGTCCGGCACGGCGTCTGT
GGTCTGCCTGCTCAATAACTTTTACCCTAGGGAAGCTAAAGTCCAATGG
AAAGTGGATAACGCCCTGCAGTCAGGAAACAGCCAGGAATCGGTTACCG
AACAGGACAGCAAGGACAGCACTTACTCCTTGTCGTCGACTCTTACTCT
GAGCAAGGCCGATTACGAGAAGCACAAGGTCTACGCCTGCGAGGTCACC
CATCAGGGACTCTCGTCCCCGGTGACCAAATCCTTCAATAGAGGCGAAT
GC (SEQ ID NO: 84)

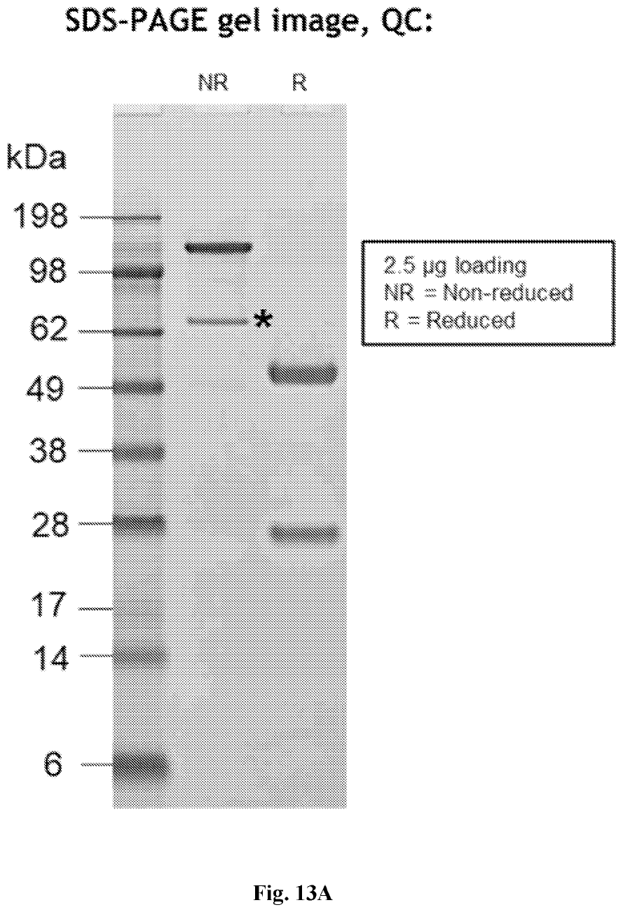

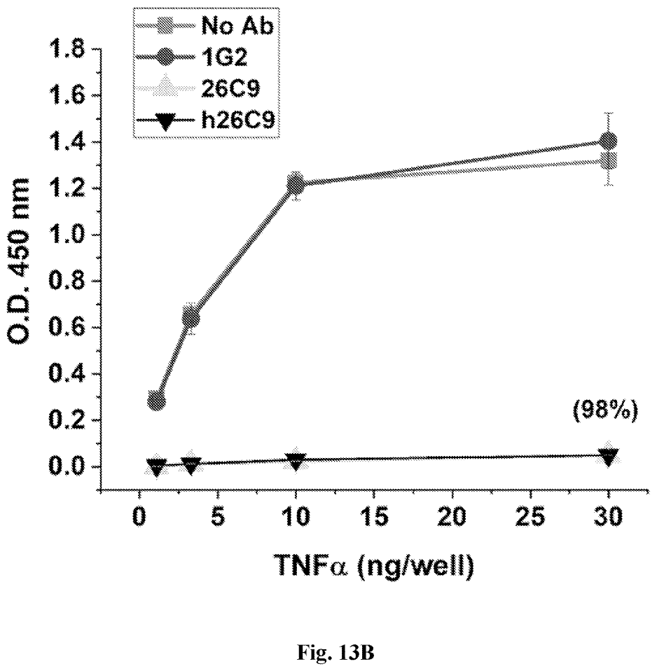

Referring to FIG. 13B, it was confirmed that h26C9 retains the potent inhibition of TNFα binding to sTNFR2 of the parent rat monoclonal antibody 26C9 in the static assay.

Human IgG4 subclass is known to spontaneously dissociated into monomers by cis disulfide bond formation (Liu et al., MAbs. 2012 January-February; 4(1):17-23). Referring to FIG. 13A, h26C9, which is of human IgG4 subclass, was indeed found to dissociated into monomers, as indicated by the band indicated by the asterisk (*) in the non-reduced lane.

To solve the cis disulfide bond formation and to increase the stability of the h26C9 IgG4 recombinant antibody, the present study further modified the h26C9 antibody by introducing several amino acid residue changes into the heavy chain of h26C9. The result antibody is designated stabilized h26C9 or d26C9.

The amino acid sequence and DNA sequence of the d26C9 heavy chain is set forth below (the light chain sequences are the same as those of the original h26C9 antibody):

d26C9 heavy chain amino acid sequence:
QVQLKESGPGILQPSQTLSLTCTFSGFSLSTYGVGVGWIRQPSGKGLEWLANIWWD
DDKYYNPSLKNRLTISKDTSNNQVFLKITNVDTADTATYYCARIDGTRYFDYWGQ
GVMVSVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYGP
PCPPCPAPEAAGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNY
KTTPPVLDSDGSFFLYSKLTVDKSRWQEGNVFSCSVMHEALHNHYTQKSLSLSLG
K (SEQ ID NO: 85) (Note: the highlighted amino acid residues are the
modifications introduced in the original h26C9 heavy chain.)
d26C9 heavy chain DNA sequence:
CAAGTGCAGCTGAAGGAGAGCGGCCCCGGCATCCTGCAGCCTTCTCAGACCCT
GAGCCTGACCTGCACCTTCAGCGGCTTCAGCCTGAGCACCTACGGCGTGGGCG
TGGGCTGGATCAGACAGCCTAGCGGCAAGGGCCTGGAGTGGCTGGCCAACATC
TGGTGGGACGACGACAAGTACTACAACCCTAGCCTGAAGAACAGACTGACGA
TTAGCAAGGACACAAGCAACAACCAAGTGTTCCTGAAGATCACCAACGTGGAC
ACCGCCGACACCGCCACCTACTACTGCGCTAGAATCGACGGCACAAGATACTT
CGACTACTGGGGCCAAGGCGTGATGGTGAGCGTGAGCAGCGCAAGCACCAAG
GGTCCTAGCGTGTTCCCTTTAGCCCCCTGCAGCAGAAGCACAAGCGAGAGCAC
CGCCGCCCTGGGCTGCCTAGTTAAGGACTACTTCCCTGAGCCCGTGACGGTCTC
CTGGAACAGTGGTGCGCTCACAAGCGGCGTGCACACCTTCCCCGCCGTGCTGC
AGAGCAGCGGCCTGTACAGCCTGAGCAGCGTGGTGACCGTCCCTAGCAGCAGC
CTGGGCACCAAGACCTACACCTGCAACGTGGACCACAAGCCTAGCAACACCAA
GGTGGACAAAAGAGTTGAGAGTAAATACGGCCCGCCTTGTCCTCCTTGCCCGG
CCCCCGAAGCCGCCGGAGGCCCTAGCGTTTTCCTGTTCCCCCCCAAGCCCAAG
GACACCCTGATGATCAGCAGAACCCCTGAGGTGACCTGCGTGGTGGTGGACGT
GAGCCAAGAGGACCCTGAGGTGCAGTTCAACTGGTACGTGGACGGCGTGGAG
GTGCACAACGCCAAGACCAAGCCTAGAGAGGAGCAGTTCAACAGCACCTACA
GAGTGGTGAGCGTGCTGACCGTGCTGCACCAAGACTGGCTGAACGGCAAGGA
GTACAAGTGCAAGGTGAGCAACAAGGGCCTGCCGAGCAGCATCGAAAAGACA
ATAAGCAAGGCCAAAGGGCAGCCTAGAGAGCCCCAAGTGTACACCCTGCCCC
CTAGCCAAGAGGAGATGACCAAGAACCAAGTGAGCCTGACTTGCCTCGTGAA
GGGATTCTACCCTAGCGACATCGCCGTGGAGTGGGAGAGCAACGGGCAGCCTG
AGAACAACTACAAGACCACCCCCCCCGTGCTGGACAGCGACGGCAGCTTCTTC
CTGTACAGCAAGCTGACCGTGGACAAGAGCAGATGGCAAGAGGGCAACGTGT
TCAGCTGCAGCGTGATGCACGAGGCCCTGCACAACCACTACACACAGAAGAGC
CTGAGCCTGAGCCTGGGCAAG (SEQ ID NO: 86)

Specifically, in the heavy chain of d26C9 antibody, the first (serine to proline (S to P)) modification was introduced to stabilize disulfide bonds in the trans orientation and therefore the recombinant antibody heterodimer. The second (glutamate-phenylalanine (EF) to alanine-alanine (AA)) modification further weakened the IgG4 Fc receptor binding activity. IgG4 already has weak Fc receptor binding activity and the second modification almost completely abolishes this binding. The third (arginine to lysin (R to K)) modification stabilizes the IgG4 CH3 domain association.

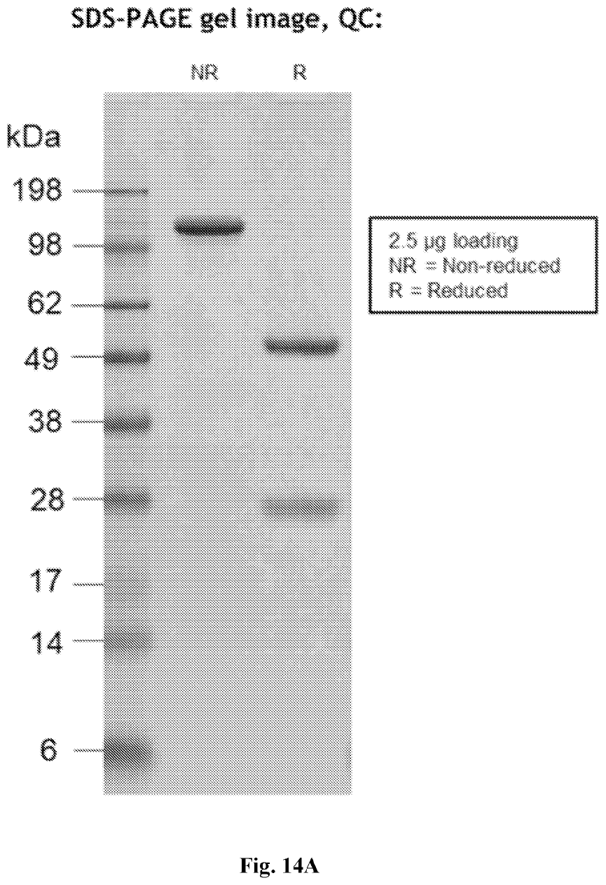

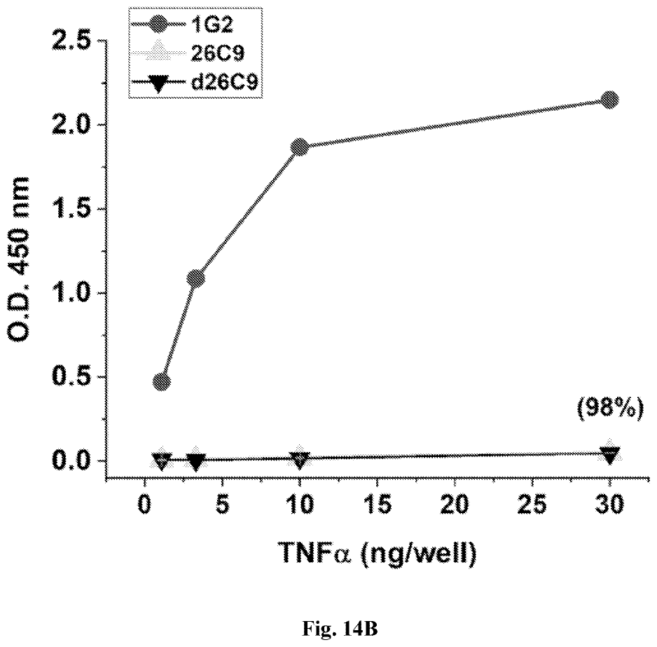

Referring to FIG. 14A, unlike the original humanized antibody h26C9, d26C9 does not spontaneously dissociate into monomers. Furthermore, d26C9 retains the potent inhibition of TNFα binding to sTNFR2 of the rat antibody 26C9 from which d26C9 derives.

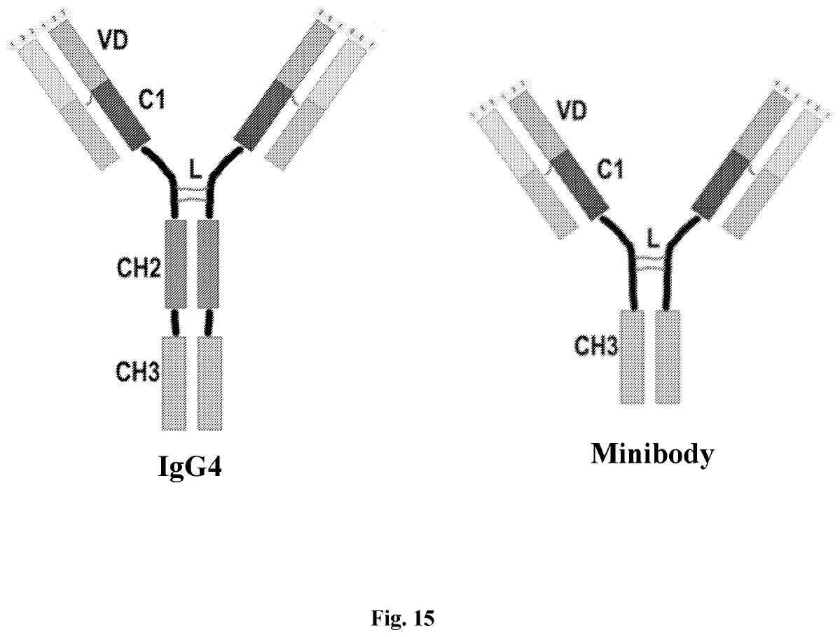

In some embodiments, the d26C9 modifications serve four purposes. 1) Stabilize the recombinant monoclonal antibody in the heterodimeric form for manufacturing that meets FDA-acceptable lot-to-lot consistency standards for a therapeutic agent. 2) Generate a purely inhibitory recombinant antibody whose biologic activity is limited to blocking the binding of TNFα to TNFR2. 3) Stabilize the association of the IgG4 CH3 domains in the heterodimer to serve as a platform for development of a novel type of mini antibody that depends on CH3 association to allow formation of disulfide bonding (see e.g., FIG. 15), enabling removal of the CH2 domain to generate a small diffusible nonantigenic antibody form to investigate as a therapeutic TNFR2 blocking agent specifically, a therapeutic platform generally. 4) To demonstrate that manipulations c-terminal to the antigen binding (VD, C1) and disulfide linker domains of humanized 26C9 do not have negative effects on TNFR2 blockade.

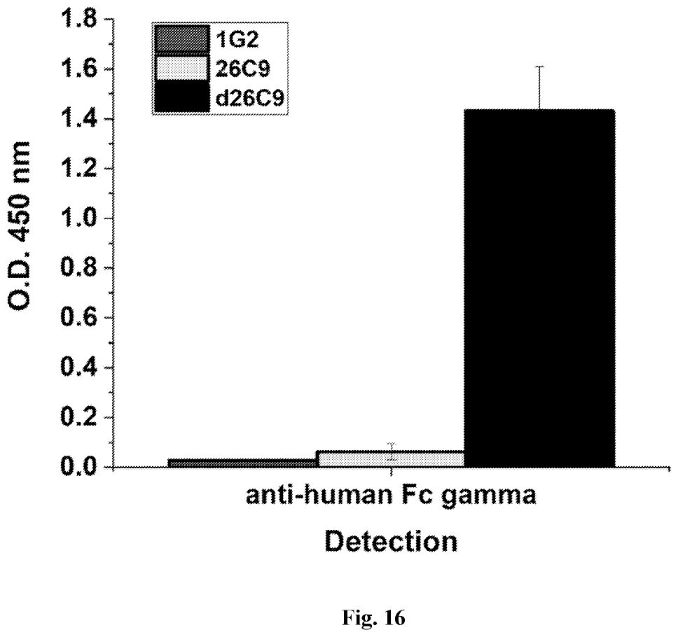

Referring to FIG. 16, to directly confirm retention of antibody binding to sTNFR2 and species immunoglobulin conversion an ELISA was performed with immobilized sTNFR2-sig1 with rat monoclonal 1G2, the original rat 26C9, and humanized/stabilized d26C9 IgG4 antibody. Detection of monoclonal antibody binding was done using anti-human Fc gamma (Jackson Immunoresearch, cat #109-036-098). Humanized/stabilized d26C9 IgG4 bound to STNFR2 was detected by anti-human Fc gamma-HRP, confirming d26C9 is both humanized and able to bind sTNFR2 by a direct independent method.

### ENUMERATED EMBODIMENTS

The following enumerated embodiments are provided, the numbering of which is not to be construed as designating levels of importance:

Embodiment 1: An antibody comprising a heavy chain polypeptide and a light chain polypeptide, wherein
the heavy chain polypeptide comprises one or more of the following:
(a) a CDR1 comprising an amino acid sequence of SEQ ID NO:1; a CDR2 comprising an amino acid sequence of SEQ ID NO:2; and a CDR3 comprising an amino acid sequence of SEQ ID NO:3;
(b) a CDR1 comprising an amino acid sequence of SEQ ID NO:7; a CDR2 comprising an amino acid sequence of SEQ ID NO:8; and a CDR3 comprising an amino acid sequence of SEQ ID NO:9;
(c) a CDR1 comprising an amino acid sequence of SEQ ID NO:13; a CDR2 comprising an amino acid sequence of SEQ ID NO:14; and a CDR3 comprising an amino acid sequence of SEQ ID NO:15;
(d) a CDR1 comprising an amino acid sequence of SEQ ID NO:18; a CDR2 comprising an amino acid sequence of SEQ ID NO:19; and a CDR3 comprising an amino acid sequence of SEQ ID NO:20;
(e) a CDR1 comprising an amino acid sequence of SEQ ID NO:23; a CDR2 comprising an amino acid sequence of SEQ ID NO:24; and a CDR3 comprising an amino acid sequence of SEQ ID NO:25,
   and
   the light chain polypeptide comprises one or more of the following:
(a) CDR1 comprising an amino acid sequence of SEQ ID NO:4; a CDR2 comprising an amino acid sequence of SEQ ID NO:5; and a CDR3 comprising an amino acid sequence of SEQ ID NO:6;
(b) a CDR1 comprising an amino acid sequence of SEQ ID NO: 10; a CDR2 comprising an amino acid sequence of SEQ ID NO:11; and a CDR3 comprising an amino acid sequence of SEQ ID NO:12;
(c) a CDR1 comprising an amino acid sequence of SEQ ID NO:4; a CDR2 comprising an amino acid sequence of SEQ ID NO: 16; and a CDR3 comprising an amino acid sequence of SEQ ID NO:17;
(d) a CDR1 comprising an amino acid sequence of SEQ ID NO:4; a CDR2 comprising an amino acid sequence of SEQ ID NO:21; and a CDR3 comprising an amino acid sequence of SEQ ID NO:22;
(e) a CDR1 comprising an amino acid sequence of SEQ ID NO:26; a CDR2 comprising an amino acid sequence of SEQ ID NO:27; and a CDR3 comprising an amino acid sequence of SEQ ID NO:28.

Embodiment 2: The antibody of Embodiment 1, comprising one or more of the following:
(a) a heavy chain polypeptide comprising a CDR1 comprising an amino acid sequence of SEQ ID NO:1, a CDR2 comprising an amino acid sequence of SEQ ID NO:2, and a CDR3 comprising an amino acid sequence of SEQ ID NO:3; and a light chain polypeptide comprising CDR1 comprising an amino acid sequence of SEQ ID NO:4, a CDR2 comprising an amino acid sequence of SEQ ID NO:5, and a CDR3 comprising an amino acid sequence of SEQ ID NO:6;
(b) a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9; and a light chain polypeptide comprising CDR1 comprising an amino acid sequence of SEQ ID NO: 10, a CDR2 comprising an amino acid sequence of SEQ ID NO:11, and a CDR3 comprising an amino acid sequence of SEQ ID NO:12;
(c) a heavy chain polypeptide comprising a CDR1 comprising an amino acid sequence of SEQ ID NO:13, a CDR2 comprising an amino acid sequence of SEQ ID NO:14, and a CDR3 comprising an amino acid sequence of SEQ ID NO:15; and a light chain polypeptide comprising CDR1 comprising an amino acid sequence of SEQ ID NO:4, a CDR2 comprising an amino acid sequence of SEQ ID NO:16, and a CDR3 comprising an amino acid sequence of SEQ ID NO:17;
(d) a heavy chain polypeptide comprising a CDR1 comprising an amino acid sequence of SEQ ID NO:18, a CDR2 comprising an amino acid sequence of SEQ ID NO:19, and a CDR3 comprising an amino acid sequence of SEQ ID NO:20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22;
(e) a heavy chain polypeptide comprising a CDR1 comprising an amino acid sequence of SEQ ID NO:23, a CDR2 comprising an amino acid sequence of SEQ ID NO:24, and a CDR3 comprising an amino acid sequence of SEQ ID NO:25; and a light chain polypeptide comprising CDR1 comprising an amino acid sequence of SEQ ID NO:26, a CDR2 comprising an amino acid sequence of SEQ ID NO:27, and a CDR3 comprising an amino acid sequence of SEQ ID NO:28.

Embodiment 3: The antibody of anyone of Embodiments 1-2, wherein the heavy chain polypeptide comprises at least one of the following:
(a) a heavy chain variable region comprising an amino acid sequence of SEQ ID NO:29;
(b) a heavy chain variable region comprising an amino acid sequence of SEQ ID NO:31;
(c) a heavy chain variable region comprising an amino acid sequence of SEQ ID NO:33;
(d) a heavy chain variable region comprising an amino acid sequence of SEQ ID NO:35;
(e) a heavy chain variable region comprising an amino acid sequence of SEQ ID NO:37;
(f) a heavy chain variable region comprising an amino acid sequence of SEQ ID NO:87,
   the light chain polypeptide comprises at least one of the following:
(a) a light chain variable region comprising an amino acid sequence of SEQ ID NO:30;
(b) a light chain variable region comprising an amino acid sequence of SEQ ID NO:32;
(c) a light chain variable region comprising an amino acid sequence of SEQ ID NO:34;
(d) a light chain variable region comprising an amino acid sequence of SEQ ID NO:36;
(e) a light chain variable region comprising an amino acid sequence of SEQ ID NO:38;
(f) a light chain variable region comprising an amino acid sequence of SEQ ID NO:88.

Embodiment 4: The antibody of any one of Embodiments 1-3, comprising one or more of the following:
(a) a heavy chain polypeptide comprising a heavy chain variable region comprising an amino acid sequence of SEQ ID NO:29; and a light chain peptide comprising a light chain variable region comprising an amino acid sequence of SEQ ID NO:30;
(b) a heavy chain polypeptide comprising a heavy chain variable region comprising an amino acid sequence of SEQ ID NO:31; and a light chain peptide comprising a light chain variable region comprising an amino acid sequence of SEQ ID NO:32;
(c) a heavy chain polypeptide comprising a heavy chain variable region comprising an amino acid sequence of SEQ ID NO:33; and a light chain peptide comprising a light chain variable region comprising an amino acid sequence of SEQ ID NO:34;
(d) a heavy chain polypeptide comprising a heavy chain variable region comprising an amino acid sequence of SEQ ID NO:35; and a light chain peptide comprising a light chain variable region comprising an amino acid sequence of SEQ ID NO:36;
(e) a heavy chain polypeptide comprising a heavy chain variable region comprising an amino acid sequence of SEQ ID NO:37; and a light chain peptide comprising a light chain variable region comprising an amino acid sequence of SEQ ID NO:38;
(f) a heavy chain polypeptide comprising a heavy chain variable region comprising an amino acid sequence of SEQ ID NO:87; a light chain peptide comprising a light chain variable region comprising an amino acid sequence of SEQ ID NO:88.

Embodiment 5: The antibody of any one of Embodiments 1-4, wherein the antibody binds to the extracellular portion of tumor necrosis factor receptor 2 (TNFR2).

Embodiment 6: The antibody of any one of Embodiments 1-5, wherein the antibody binds to soluble tumor necrosis factor receptor 2 (sTNFR2).

Embodiment 7: The antibody of any one of Embodiments 5-6, wherein the TNFR2 and/or the sTNFR2 are human TNFR2 and/or human sTNFR2.

Embodiment 8: The antibody of any one of Embodiments 1-7, wherein the antibody does not bind to soluble tumor necrosis factor receptor 1 (sTNFR1).

Embodiment 9: The antibody of any one of Embodiments 1-8, which is isolated.

Embodiment 10: The antibody of any one of Embodiments 1-9, which is recombinant.

Embodiment 11: The antibody of any one of Embodiments 1-10, which is an IgG, IgM, IgA, or an antigen binding fragment thereof.

Embodiment 12: The antibody of any one of Embodiments 1-11, wherein the antibody is a Fab′, a F(ab′)2, a F(ab′)3, a monovalent scFv, a bivalent scFv, a single domain antibody, or a minibody.

Embodiment 13: The antibody of any one of Embodiments 1-12, wherein the antibody is a full length antibody.

Embodiment 14: The antibody of any one of Embodiments 1-13, wherein the antibody is a human, humanized antibody, or de-immunized antibody.

Embodiment 15: A nucleic acid encoding an antibody, comprising:
a first nucleic acid comprising one or more of the following:
(a) a first portion encoding an amino acid sequence of SEQ ID NO:1; a second portion encoding an amino acid sequence of SEQ ID NO:2; and a third portion encoding an amino acid sequence of SEQ ID NO:3;
(b) a first portion encoding an amino acid sequence of SEQ ID NO:7; a second portion encoding an amino acid sequence of SEQ ID NO:8; and a third portion encoding an amino acid sequence of SEQ ID NO:9;
(c) a first portion encoding an amino acid sequence of SEQ ID NO:13; a second portion encoding an amino acid sequence of SEQ ID NO: 14; and a third portion encoding an amino acid sequence of SEQ ID NO:15;
(e) a first portion encoding an amino acid sequence of SEQ ID NO:18; a second portion encoding an amino acid sequence of SEQ ID NO:19; and a third portion encoding an amino acid sequence of SEQ ID NO:20;
(f) a first portion encoding an amino acid sequence of SEQ ID NO:23; a second portion encoding an amino acid sequence of SEQ ID NO:24; and a third portion encoding an amino acid sequence of SEQ ID NO:25; or
   a second nucleic acid comprising one or more of the following:
(a) a first portion encoding an amino acid sequence of SEQ ID NO:4; a second portion encoding an amino acid sequence of SEQ ID NO:5; and a third portion encoding an amino acid sequence of SEQ ID NO:6;
(b) a first portion encoding an amino acid sequence of SEQ ID NO:10; a second portion encoding an amino acid sequence of SEQ ID NO: 11; and a third portion encoding an amino acid sequence of SEQ ID NO:12;
(c) a first portion encoding an amino acid sequence of SEQ ID NO:4; a second portion encoding an amino acid sequence of SEQ ID NO:16; and a third portion encoding an amino acid sequence of SEQ ID NO:17;
(d) a first portion encoding an amino acid sequence of SEQ ID NO:4; a second portion encoding an amino acid sequence of SEQ ID NO:21; and a third portion encoding an amino acid sequence of SEQ ID NO:22;
(e) a first portion encoding an amino acid sequence of SEQ ID NO:26; a second portion encoding an amino acid sequence of SEQ ID NO:27; and a third portion encoding an amino acid sequence of SEQ ID NO:28.

Embodiment 16: The nucleic acid of Embodiment 15, wherein the first nucleic acid comprises one or more of the following:
(a) a first portion comprising the sequence of SEQ ID NO:39; a second portion comprising the sequence of SEQ ID NO:40; and a third portion comprising the sequence of SEQ ID NO: 41;
(b) a first portion comprising the sequence of SEQ ID NO:45; a second portion comprising the sequence of SEQ ID NO:46; and a third portion comprising a sequence of SEQ ID NO: 47;
(c) a first portion comprising the sequence of SEQ ID NO:51; a second portion comprising the sequence of SEQ ID NO:52; and a third portion comprising the sequence of SEQ ID NO: 53;
(d) a first portion comprising the sequence of SEQ ID NO:56; a second portion comprising the sequence of SEQ ID NO:57; and a third portion comprising a sequence of SEQ ID NO: 58;
(e) a first portion comprising the sequence of SEQ ID NO:62; a second portion comprising the sequence of SEQ ID NO: 63; and a third portion comprising the sequence of SEQ ID NO: 64, or
   the second nucleic acid comprises one or more of the following:
(a) a first portion comprising the sequence of SEQ ID NO:42; a second portion comprising the sequence of SEQ ID NO:43; and a third portion comprising the sequence of SEQ ID NO: 44;
(b) a first portion comprising the sequence of SEQ ID NO:48; a second portion comprising the sequence of SEQ ID NO:49; and a third portion comprising a sequence of SEQ ID NO: 50;
(c) a first portion comprising the sequence of SEQ ID NO:42; a second portion comprising the sequence of SEQ ID NO:54; and a third portion comprising the sequence of SEQ ID NO: 55;
(d) a first portion comprising the sequence of SEQ ID NO:59; a second portion comprising the sequence of SEQ ID NO:60; and a third portion comprising a sequence of SEQ ID NO: 61;
(e) a first portion comprising the sequence of SEQ ID NO:65; a second portion comprising the sequence of SEQ ID NO:66; and a third portion comprising the sequence of SEQ ID NO: 67.

Embodiment 17: The nucleic acid of any one of Embodiments 15-16, wherein the first nucleic acid comprises at least one of the following:
(a) a nucleic acid encoding a heavy chain variable region comprising an amino acid sequence of SEQ ID NO:29;
(b) a nucleic acid encoding a heavy chain variable region comprising an amino acid sequence of SEQ ID NO:31;
(c) a nucleic acid encoding a heavy chain variable region comprising an amino acid sequence of SEQ ID NO:33;
(d) a nucleic acid encoding a heavy chain variable region comprising an amino acid sequence of SEQ ID NO:35;
(e) a nucleic acid encoding a heavy chain variable region comprising an amino acid sequence of SEQ ID NO:37;
(f) a nucleic acid encoding a heavy chain variable region comprising an amino acid sequence of SEQ ID NO:87, or
   the second nucleic acid comprises at least one of the following:
(a) a nucleic acid encoding a light chain variable region comprising an amino acid sequence of SEQ ID NO:30;
(b) a nucleic acid encoding a light chain variable region comprising an amino acid sequence of SEQ ID NO:32;
(c) a nucleic acid encoding a light chain variable region comprising an amino acid sequence of SEQ ID NO:34;
(d) a nucleic acid encoding a light chain variable region comprising an amino acid sequence of SEQ ID NO:36;
(e) a nucleic acid encoding a light chain variable region comprising an amino acid sequence of SEQ ID NO:38;
(f) a nucleic acid encoding a light chain variable region comprising an amino acid sequence of SEQ ID NO:88.

Embodiment 18: The nucleic acid of any one of Embodiments 15-17, comprising at least one of the following:
(a) a first nucleic acid comprising a nucleic acid encoding a heavy chain variable region comprising an amino acid sequence of SEQ ID NO:29; and a second nucleic acid comprising a nucleic acid encoding a light chain variable region comprising an amino acid sequence of SEQ ID NO:30;
(b) a first nucleic acid comprising a nucleic acid encoding a heavy chain variable region comprising an amino acid sequence of SEQ ID NO:31; and a second nucleic acid comprising a nucleic acid encoding a light chain variable region comprising an amino acid sequence of SEQ ID NO:32;
(c) a first nucleic acid comprising a nucleic acid encoding a heavy chain variable region comprising an amino acid sequence of SEQ ID NO:33; and a second nucleic acid comprising a nucleic acid encoding a light chain variable region comprising an amino acid sequence of SEQ ID NO:34;
(d) a first nucleic acid comprising a nucleic acid encoding a heavy chain variable region comprising an amino acid sequence of SEQ ID NO:35; and a second nucleic acid comprising a nucleic acid encoding a light chain variable region comprising an amino acid sequence of SEQ ID NO:36;
(e) a first nucleic acid comprising a nucleic acid encoding a heavy chain variable region comprising an amino acid sequence of SEQ ID NO:37; and a second nucleic acid comprising a nucleic acid encoding a light chain variable region comprising an amino acid sequence of SEQ ID NO:38.

Embodiment 19: The nucleic acid of any one of Embodiments 15-18, wherein
the first nucleic acid comprises at least one of the following:
(a) a nucleic acid comprising the sequence of SEQ ID NO:68;
(b) a nucleic acid comprising the sequence of SEQ ID NO:70;
(c) a nucleic acid comprising the sequence of SEQ ID NO:72;
(d) a nucleic acid comprising the sequence of SEQ ID NO:74;
(e) a nucleic acid comprising the sequence of SEQ ID NO:76;
(f) a nucleic acid comprising the sequence of SEQ ID NO:89, or
   the second nucleic acid comprises at least one of the following:
(a) a nucleic acid comprising the sequence of SEQ ID NO:69;
(b) a nucleic acid comprising the sequence of SEQ ID NO:71;
(c) a nucleic acid comprising the sequence of SEQ ID NO: 73;
(d) a nucleic acid comprising the sequence of SEQ ID NO:75;
(e) a nucleic acid comprising the sequence of SEQ ID NO:77;
(f) a nucleic acid comprising the sequence of SEQ ID NO:90.

Embodiment 20: The nucleic acid of any one of Embodiments 15-19, comprising at least one of the following:
(a) a first nucleic acid comprising the sequence of SEQ ID NO:68; and a second nucleic acid comprising the sequence of SEQ ID NO:69;
(b) a first nucleic acid comprising the sequence of SEQ ID NO:70; and a second nucleic acid comprising the sequence of SEQ ID NO:71;
(c) a first nucleic acid comprising the sequence of SEQ ID NO:72; and a second nucleic acid comprising the sequence of SEQ ID NO:73;
(d) a first nucleic acid comprising the sequence of SEQ ID NO:74; and a second nucleic acid comprising the sequence of SEQ ID NO:75;
(e) a first nucleic acid comprising the sequence of SEQ ID NO:76; and a second nucleic acid comprising the sequence of SEQ ID NO:77;
(f) a first nucleic acid comprising the sequence of SEQ ID NO:89; and a second nucleic acid comprising the sequence of SEQ ID NO:90.

Embodiment 21: The nucleic acid of any one of Embodiments 15-20, wherein the nucleic acid comprises an expression vector.

Embodiment 22: An antibody pair, comprising a first antibody and a second antibody, wherein the first antibody and the second antibody each independently comprises:
(a) a heavy chain polypeptide comprising a CDR1 comprising an amino acid sequence of SEQ ID NO:1, a CDR2 comprising an amino acid sequence of SEQ ID NO:2, and a CDR3 comprising an amino acid sequence of SEQ ID NO:3; and a light chain polypeptide comprising CDR1 comprising an amino acid sequence of SEQ ID NO:4, a CDR2 comprising an amino acid sequence of SEQ ID NO:5, and a CDR3 comprising an amino acid sequence of SEQ ID NO:6;
(b) a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9; and a light chain polypeptide comprising CDR1 comprising an amino acid sequence of SEQ ID NO:10, a CDR2 comprising an amino acid sequence of SEQ ID NO:11, and a CDR3 comprising an amino acid sequence of SEQ ID NO:12;
(c) a heavy chain polypeptide comprising a CDR1 comprising an amino acid sequence of SEQ ID NO:13, a CDR2 comprising an amino acid sequence of SEQ ID NO:14, and a CDR3 comprising an amino acid sequence of SEQ ID NO:15; and a light chain polypeptide comprising CDR1 comprising an amino acid sequence of SEQ ID NO:4, a CDR2 comprising an amino acid sequence of SEQ ID NO:16, and a CDR3 comprising an amino acid sequence of SEQ ID NO:17;
(d) a heavy chain polypeptide comprising a CDR1 comprising an amino acid sequence of SEQ ID NO:18, a CDR2 comprising an amino acid sequence of SEQ ID NO:19, and a CDR3 comprising an amino acid sequence of SEQ ID NO:20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22;
(e) a heavy chain polypeptide comprising a CDR1 comprising an amino acid sequence of SEQ ID NO:23, a CDR2 comprising an amino acid sequence of SEQ ID NO:24, and a CDR3 comprising an amino acid sequence of SEQ ID NO:25; and a light chain polypeptide comprising CDR1 comprising an amino acid sequence of SEQ ID NO:26, a CDR2 comprising an amino acid sequence of SEQ ID NO:27, and a CDR3 comprising an amino acid sequence of SEQ ID NO:28, and
   wherein the first antibody and the second antibody does not compete for the same epitope on sTNFR2.

Embodiment 23: The antibody pair of Embodiment 22, wherein
the first antibody comprises a heavy chain polypeptide comprising a CDR1 comprising an amino acid sequence of SEQ ID NO:18, a CDR2 comprising an amino acid sequence of SEQ ID NO: 19, and a CDR3 comprising an amino acid sequence of SEQ ID NO: 20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22, and
the second antibody comprises a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO: 9; and a light chain polypeptide comprising CDR1 comprising an amino acid sequence of SEQ ID NO:10, a CDR2 comprising an amino acid sequence of SEQ ID NO:11, and a CDR3 comprising an amino acid sequence of SEQ ID NO:12.

Embodiment 24: The antibody pair of any one of Embodiments 22-23, wherein the first antibody and the second antibody each independently comprises one of the following:
(a) a heavy chain polypeptide comprising a heavy chain variable region comprising an amino acid sequence of SEQ ID NO:29; and a light chain peptide comprising a light chain variable region comprising an amino acid sequence of SEQ ID NO:30;
(b) a heavy chain polypeptide comprising a heavy chain variable region comprising an amino acid sequence of SEQ ID NO:31; and a light chain peptide comprising a light chain variable region comprising an amino acid sequence of SEQ ID NO:32;
(c) a heavy chain polypeptide comprising a heavy chain variable region comprising an amino acid sequence of SEQ ID NO:33; and a light chain peptide comprising a light chain variable region comprising an amino acid sequence of SEQ ID NO:34;
(d) a heavy chain polypeptide comprising a heavy chain variable region comprising an amino acid sequence of SEQ ID NO:35; and a light chain peptide comprising a light chain variable region comprising an amino acid sequence of SEQ ID NO:36;
(e) a heavy chain polypeptide comprising a heavy chain variable region comprising an amino acid sequence of SEQ ID NO:37; and a light chain peptide comprising a light chain variable region comprising an amino acid sequence of SEQ ID NO:38;
(f) a heavy chain polypeptide comprising a heavy chain variable region comprising an amino acid sequence of SEQ ID NO:87; and a light chain peptide comprising a light chain variable region comprising an amino acid sequence of SEQ ID NO:88.

Embodiment 25: The antibody pair of any one of Embodiments 22-24, wherein
the first antibody comprises a heavy chain polypeptide comprising a heavy chain variable region comprising an amino acid sequence of SEQ ID NO:35; and a light chain peptide comprising a light chain variable region comprising an amino acid sequence of SEQ ID NO: 36, or the first antibody comprises a heavy chain polypeptide comprising a heavy chain variable region comprising an amino acid sequence of SEQ ID NO:87; and a light chain peptide comprising a light chain variable region comprising an amino acid sequence of SEQ ID NO:88, and
the second antibody comprises a heavy chain polypeptide comprising a heavy chain variable region comprising an amino acid sequence of SEQ ID NO:31; and a light chain peptide comprising a light chain variable region comprising an amino acid sequence of SEQ ID NO: 32.

Embodiment 26: The antibody pair of any one of Embodiments 22-25, wherein the antibody pair quantifies sTNFR2 in a sample, and wherein the first antibody captures STNFR2 in the sample and the second antibody quantifies the sTNFR2 captured by the first antibody.

Embodiment 27: A method of quantifying sTNFR2 in a sample using the antibody pair of any one of Embodiments 22-26, comprising:
capturing sTNFR2 in the sample with the first antibody; and
quantifying sTNFR2 captured by the first antibody with the second antibody.

Embodiment 28: The method of Embodiment 27, wherein the method of quantifying is a western blotting-based method, an enzyme-linked immunoassay (ELISA)-based method, or combinations thereof.

Embodiment 29: The method of Embodiment 27-28, wherein the sample is a serum sample, a plasma sample, a urine sample, or combinations thereof.

Embodiment 30: The method of any one of Embodiments 27-29, wherein the sample is a sample from a subject suspected to have a disease or disorder indicated by altered levels of sTNFR2, and the quantification of sTNFR2 in the sample diagnosis the disease or disorder.

Embodiment 31: The method of Embodiment 30, wherein the disease is Kawasaki disease (K_{D}) or Kawasaki-like syndrome (KLS).

Embodiment 32: The method of Embodiments 27-31, wherein the sample is a sample from a subject confirmed to have a disease or disorder involving altered levels of sTNFR2, and wherein the quantification of sTNFR2 predicts outcomes of the disease or disorder or monitors the state of the disease or disorder during therapies.

Embodiment 33: The method of Embodiment 32, wherein the disease or disorder is lupus nephritis, Behçet's disease, Sjogren's syndrome, rheumatoid arthritis and juvenile chronic arthritis, inflammatory bowel diseases, sarcoidosis, Grave's disease, myocardial infarction, congestive heart failure, progressive diabetic kidney disease in subjects with type 2 diabetes mellitus, erythropoietin resistance in setting of hemodialysis, steatohepatitis, hepatitis C virus liver fibrosis, post-COVID-19 multisystem inflammatory syndrome (MIS-C) in children, presence and/or prognosis of infections including sepsis, malaria, COVID-19, or HIV.

Embodiment 34: A method of diagnosing in a subject a disease or disorder involving altered levels of sTNFR2, the method comprising:
capturing sTNFR2 in a sample from the subject with the first antibody of any one of Embodiments 22-26;
quantifying sTNFR2 captured by the first antibody with the second antibody of any one of Embodiments 22-26;
wherein, if the sTNFR2 level quantified by the first antibody and the second antibody is equal to or higher than a first predetermined level, the subject is diagnosed as having the disease or disorder, and
wherein, if the sTNFR2 level quantified by the first antibody and the second antibody is lower than a first predetermined level, the subject is diagnosed as not having the disease or disorder.

Embodiment 35: The method of Embodiment 34, wherein the method of quantifying is a western blotting-based method, an enzyme-linked immunoassay (ELISA)-based method, or combinations thereof.

Embodiment 36: The method of any one of Embodiments 34-35, wherein the sample is a serum sample, a plasma sample, a urine sample, or combinations thereof.

Embodiment 37: The method of any one of Embodiments 34-36, wherein the disease is Kawasaki disease (K_{D}) or Kawasaki-like syndrome (KLS).

Embodiment 38: A method of monitoring progression and/or state of a disease or disorder involving altered levels of sTNFR2 in a subject diagnosed with the disease or disorder, the method comprising:
obtaining subject's samples at two or more time points after diagnosis of the disease or disorder in the subject;
capturing sTNFR2 in each obtained sample with the first antibody of any one of Embodiments 22-26;
quantifying sTNFR2 captured by the first antibody with the second antibody of any one of Embodiments 22-26;
comparing the sTNFR2 levels quantified by the first antibody and the second antibody in the obtained subject's samples;
wherein the comparing step allows for monitoring progression and/or state of the disease or disorder in the subject.

Embodiment 39: The method of Embodiment 38, wherein at least one of the two or more time points is a time point where the subject is receiving therapeutic treatment for the disease or disorder, and wherein the comparing step allows for monitoring therapeutic outcome of the therapeutic treatment in the subject.

Embodiment 40: The method of any one of Embodiments 38-39, wherein the method of quantifying is a western blotting-based method, an enzyme-linked immunoassay (ELISA)-based method, or combinations thereof.

Embodiment 41: The method of any one of Embodiments 38-40, wherein each sample is independently a serum sample, a plasma sample, a urine sample, or combinations thereof.

Embodiment 42: The method of any one of Embodiments 38-41, wherein the disease or disorder is lupus nephritis, Behçet's disease, Sjogren's syndrome, rheumatoid arthritis and juvenile chronic arthritis, inflammatory bowel diseases, sarcoidosis, Grave's disease, myocardial infarction, congestive heart failure, progressive diabetic kidney disease in subjects with type 2 diabetes mellitus, erythropoietin resistance in setting of hemodialysis, steatohepatitis, hepatitis C virus liver fibrosis, post-COVID-19 multisystem inflammatory syndrome (MIS-C) in children, presence and/or prognosis of infections including sepsis, malaria, COVID-19, or HIV.

Embodiment 43: A method of treating, ameliorating, and/or preventing Kawasaki disease or Kawasaki-like syndrome in a subject in need thereof, comprising:
quantifying sTNFR2 level in a sample from the subject, comprising capturing sTNFR in the sample with the first antibody of any one of Embodiments 16-20, and quantifying the STNFR2 captured by the first antibody with the second antibody of any one of Embodiments 16-20;
administering to the subject an effective amount of a medication for treating Kawasaki disease or Kawasaki-like syndrome if the sTNFR2 level quantified by the first antibody and the second antibody is equal to or higher than a first predetermined level.

Embodiment 44: The method of Embodiment 43, wherein the sample is a serum sample, a plasma sample, a urine sample, or combinations thereof.

Embodiment 45: The method of any one of Embodiments 43-44, wherein the method further comprises:
quantifying C—C motif chemokine ligand 1 (Ccl1) level in the sample;
quantifying C—C motif chemokine ligand 2 (Ccl2) level in the sample; and
quantifying C—X—C motif chemokine 11 (Cxc11) level in the sample, and
wherein the subject is administered with the medication for treating Kawasaki disease or Kawasaki-like syndrome if the sTNFR2 level quantified by the first antibody and the second antibody is equal to or higher than a first predetermined level, and if at least two of the following are true:
(a) the quantified Ccl1 level is higher than a second predetermined level,
(b) the quantified Ccl2 level is higher than a third predetermined level,
(c) the quantified Cxc11 level is higher than a fourth predetermined level.

Embodiment 46: The method of any one of Embodiments 43-45, wherein the medication comprises an intravenous immunoglobulin (IVIG), aspirin, a steroid, or combinations thereof.

Embodiment 47: A method of treating, ameliorating and/or preventing an inflammatory disease or disorder in a subject in need thereof, the method comprises:
administering to the subject an effective amount of an antibody of any one of Embodiments 1 14,
wherein the inflammatory disease is caused by or involves overactive TNFα-TNFR2 signaling.

Embodiment 48: The method of Embodiment 47, wherein the disease or disorder is at least one selected from the group consisting of lupus nephritis, plaque psoriasis, psoriatic arthritis, ankylosing spondylitis, Crohn's disease, ulcerative colitis, hidradenitis, uveitis, Kawasaki disease, Behçet's disease, Sjogren's syndrome, rheumatoid arthritis and juvenile chronic arthritis, juvenile rheumatoid arthritis with macrophage activation syndrome, inflammatory bowel diseases, sarcoidosis, Grave's disease, myocardial infarction, congestive heart failure, progressive diabetic kidney disease in subjects with type 2 diabetes mellitus, steatohepatitis, hepatitis C virus liver fibrosis, post-COVID-19 multisystem inflammatory syndrome (MIS-C) in children and adults (MIS-A), bacterial sepsis, interstitial lung disease, cerebral malaria, acute respiratory distress syndrome (ARDS) or diffuse alveolar hemorrhage (DAH) associated with viral infections comprising COVID-19, and bacterial infections comprising leptospirosis.

Embodiment 49: The method of any one of Embodiments 47-48, wherein the method reduces a level of interferon γ (IFN-γ) in the subject.

Embodiment 50: The method of any one of Embodiments 47-49, wherein the method reduces an expression of IFN-γ by the peripheral blood mononuclear cells (PBMCs).

Embodiment 51: The method of any one of Embodiments 47-50, wherein the subject is a mammal, optionally a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. An antibody comprising a heavy chain polypeptide and a light chain polypeptide, wherein
the heavy chain polypeptide comprises one or more of the following:
(a) a CDR1 comprising an amino acid sequence of SEQ ID NO:1; a CDR2 comprising an amino acid sequence of SEQ ID NO:2; and a CDR3 comprising an amino acid sequence of SEQ ID NO:3;
(b) a CDR1 comprising an amino acid sequence of SEQ ID NO:7; a CDR2 comprising an amino acid sequence of SEQ ID NO:8; and a CDR3 comprising an amino acid sequence of SEQ ID NO:9;
(c) a CDR1 comprising an amino acid sequence of SEQ ID NO:13; a CDR2 comprising an amino acid sequence of SEQ ID NO:14; and a CDR3 comprising an amino acid sequence of SEQ ID NO:15;
(d) a CDR1 comprising an amino acid sequence of SEQ ID NO:18; a CDR2 comprising an amino acid sequence of SEQ ID NO:19; and a CDR3 comprising an amino acid sequence of SEQ ID NO:20;
(e) a CDR1 comprising an amino acid sequence of SEQ ID NO:23; a CDR2 comprising an amino acid sequence of SEQ ID NO:24; and a CDR3 comprising an amino acid sequence of SEQ ID NO:25, and
the light chain polypeptide comprises one or more of the following:
(a) a CDR1 comprising an amino acid sequence of SEQ ID NO:4; a CDR2 comprising an amino acid sequence of SEQ ID NO:5; and a CDR3 comprising an amino acid sequence of SEQ ID NO:6;
(b) a CDR1 comprising an amino acid sequence of SEQ ID NO:10; a CDR2 comprising an amino acid sequence of SEQ ID NO:11; and a CDR3 comprising an amino acid sequence of SEQ ID NO:12;
(c) a CDR1 comprising an amino acid sequence of SEQ ID NO:4; a CDR2 comprising an amino acid sequence of SEQ ID NO:16; and a CDR3 comprising an amino acid sequence of SEQ ID NO:17;
(d) a CDR1 comprising an amino acid sequence of SEQ ID NO:4; a CDR2 comprising an amino acid sequence of SEQ ID NO:21; and a CDR3 comprising an amino acid sequence of SEQ ID NO:22;
(e) a CDR1 comprising an amino acid sequence of SEQ ID NO:26; a CDR2 comprising an amino acid sequence of SEQ ID NO:27; and a CDR3 comprising an amino acid sequence of SEQ ID NO:28.

2. The antibody of claim 1, comprising one or more of the following:
(a) a heavy chain polypeptide comprising a CDR1 comprising an amino acid sequence of SEQ ID NO:1, a CDR2 comprising an amino acid sequence of SEQ ID NO:2, and a CDR3 comprising an amino acid sequence of SEQ ID NO:3; and
a light chain polypeptide comprising CDR1 comprising an amino acid sequence of SEQ ID NO: 4, a CDR2 comprising an amino acid sequence of SEQ ID NO:5, and a CDR3 comprising an amino acid sequence of SEQ ID NO:6;
(b) a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9; and
a light chain polypeptide comprising CDR1 comprising an amino acid sequence of SEQ ID NO: 10, a CDR2 comprising an amino acid sequence of SEQ ID NO:11, and a CDR3 comprising an amino acid sequence of SEQ ID NO:12;
(c) a heavy chain polypeptide comprising a CDR1 comprising an amino acid sequence of SEQ ID NO:13, a CDR2 comprising an amino acid sequence of SEQ ID NO: 14, and a CDR3 comprising an amino acid sequence of SEQ ID NO:15; and
a light chain polypeptide comprising CDR1 comprising an amino acid sequence of SEQ ID NO: 4, a CDR2 comprising an amino acid sequence of SEQ ID NO:16, and a CDR3 comprising an amino acid sequence of SEQ ID NO:17;
(d) a heavy chain polypeptide comprising a CDR1 comprising an amino acid sequence of SEQ ID NO:18, a CDR2 comprising an amino acid sequence of SEQ ID NO:19, and a CDR3 comprising an amino acid sequence of SEQ ID NO:20; and
a light chain polypeptide comprising CDR1 comprising an amino acid sequence of SEQ ID NO: 4, a CDR2 comprising an amino acid sequence of SEQ ID NO:21, and a CDR3 comprising an amino acid sequence of SEQ ID NO:22;
(e) a heavy chain polypeptide comprising a CDR1 comprising an amino acid sequence of SEQ ID NO:23, a CDR2 comprising an amino acid sequence of SEQ ID NO:24, and a CDR3 comprising an amino acid sequence of SEQ ID NO:25; and
a light chain polypeptide comprising CDR1 comprising an amino acid sequence of SEQ ID NO: 26, a CDR2 comprising an amino acid sequence of SEQ ID NO:27, and a CDR3 comprising an amino acid sequence of SEQ ID NO:28.

3. The antibody of claim 1, wherein
the heavy chain polypeptide comprises at least one of the following:
(a) a heavy chain variable region comprising an amino acid sequence of SEQ ID NO:29;
(b) a heavy chain variable region comprising an amino acid sequence of SEQ ID NO:31;
(c) a heavy chain variable region comprising an amino acid sequence of SEQ ID NO:33;
(d) a heavy chain variable region comprising an amino acid sequence of SEQ ID NO:35;
(e) a heavy chain variable region comprising an amino acid sequence of SEQ ID NO:37;
(f) a heavy chain variable region comprising an amino acid sequence of SEQ ID NO:87;
the light chain polypeptide comprises at least one of the following:
(a) a light chain variable region comprising an amino acid sequence of SEQ ID NO:30;
(b) a light chain variable region comprising an amino acid sequence of SEQ ID NO:32;
(c) a light chain variable region comprising an amino acid sequence of SEQ ID NO:34;
(d) a light chain variable region comprising an amino acid sequence of SEQ ID NO:36;
(e) a light chain variable region comprising an amino acid sequence of SEQ ID NO:38;
(f) a light chain variable region comprising an amino acid sequence of SEQ ID NO:88.

4. The antibody of claim 1, comprising one or more of the following:
(a) a heavy chain polypeptide comprising a heavy chain variable region comprising an amino acid sequence of SEQ ID NO:29; and
a light chain peptide comprising a light chain variable region comprising an amino acid sequence of SEQ ID NO:30;
(b) a heavy chain polypeptide comprising a heavy chain variable region comprising an amino acid sequence of SEQ ID NO:31; and
a light chain peptide comprising a light chain variable region comprising an amino acid sequence of SEQ ID NO:32;
(c) a heavy chain polypeptide comprising a heavy chain variable region comprising an amino acid sequence of SEQ ID NO:33; and
a light chain peptide comprising a light chain variable region comprising an amino acid sequence of SEQ ID NO:34;
(d) a heavy chain polypeptide comprising a heavy chain variable region comprising an amino acid sequence of SEQ ID NO:35; and
a light chain peptide comprising a light chain variable region comprising an amino acid sequence of SEQ ID NO:36;
(e) a heavy chain polypeptide comprising a heavy chain variable region comprising an amino acid sequence of SEQ ID NO:37;
a light chain peptide comprising a light chain variable region comprising an amino acid sequence of SEQ ID NO:38;
(f) a heavy chain polypeptide comprising a heavy chain variable region comprising an amino acid sequence of SEQ ID NO:87;
a light chain peptide comprising a light chain variable region comprising an amino acid sequence of SEQ ID NO:88.

5. The antibody of claim 1, wherein at least one of the following applies:
(a) the antibody binds to the extracellular portion of tumor necrosis factor receptor 2 (TNFR2);
(b) the antibody binds to soluble tumor necrosis factor receptor 2 (sTNFR2).

6. (canceled)

7. The antibody of claim 5, wherein at least one of the following applies:
(a) the TNFR2 is human TNFR2;
(b) the sTNFR2 is human sTNFR2.

8. (canceled)

9. The antibody of claim 1, wherein at least one of the following applies:
(a) the antibody is isolated;
(b) the antibody is recombinant;
(c) an IgG, IgM, IgA, or an antigen binding fragment thereof;
(d) the antibody is a Fab′, a F(ab′)2, a F(ab′)3, a monovalent scFv, a bivalent scFv, a single domain antibody, or a minibody;
(e) the antibody is a full length antibody;
(f) the antibody is a human antibody, a humanized antibody, or a de-immunized antibody.

10-14. (canceled)

15. A nucleic acid encoding an antibody, comprising:
a first nucleic acid comprising one or more of the following:
(a) a first portion encoding an amino acid sequence of SEQ ID NO:1; a second portion encoding an amino acid sequence of SEQ ID NO:2; and a third portion encoding an amino acid sequence of SEQ ID NO:3;
(b) a first portion encoding an amino acid sequence of SEQ ID NO:7; a second portion encoding an amino acid sequence of SEQ ID NO:8; and a third portion encoding an amino acid sequence of SEQ ID NO:9;
(c) a first portion encoding an amino acid sequence of SEQ ID NO:13; a second portion encoding an amino acid sequence of SEQ ID NO: 14; and a third portion encoding an amino acid sequence of SEQ ID NO:15;
(e) a first portion encoding an amino acid sequence of SEQ ID NO: 18; a second portion encoding an amino acid sequence of SEQ ID NO:19; and a third portion encoding an amino acid sequence of SEQ ID NO:20;
(f) a first portion encoding an amino acid sequence of SEQ ID NO:23; a second portion encoding an amino acid sequence of SEQ ID NO:24; and a third portion encoding an amino acid sequence of SEQ ID NO:25; or
a second nucleic acid comprising one or more of the following:
(a) a first portion encoding an amino acid sequence of SEQ ID NO:4; a second portion encoding an amino acid sequence of SEQ ID NO:5; and a third portion encoding an amino acid sequence of SEQ ID NO:6;
(b) a first portion encoding an amino acid sequence of SEQ ID NO: 10; a second portion encoding an amino acid sequence of SEQ ID NO:11; and a third portion encoding an amino acid sequence of SEQ ID NO:12;
(c) a first portion encoding an amino acid sequence of SEQ ID NO:4; a second portion encoding an amino acid sequence of SEQ ID NO:16; and a third portion encoding an amino acid sequence of SEQ ID NO:17;
(d) a first portion encoding an amino acid sequence of SEQ ID NO:4; a second portion encoding an amino acid sequence of SEQ ID NO:21; and a third portion encoding an amino acid sequence of SEQ ID NO:22;
(e) a first portion encoding an amino acid sequence of SEQ ID NO:26; a second portion encoding an amino acid sequence of SEQ ID NO:27; and a third portion encoding an amino acid sequence of SEQ ID NO:28.

16. The nucleic acid of claim 15, wherein
the first nucleic acid comprises one or more of the following:
(a) a first portion comprising the sequence of SEQ ID NO:39; a second portion comprising the sequence of SEQ ID NO:40; and a third portion comprising the sequence of SEQ ID NO: 41;
(b) a first portion comprising the sequence of SEQ ID NO:45; a second portion comprising the sequence of SEQ ID NO:46; and a third portion comprising a sequence of SEQ ID NO: 47;
(c) a first portion comprising the sequence of SEQ ID NO:51; a second portion comprising the sequence of SEQ ID NO:52; and a third portion comprising the sequence of SEQ ID NO: 53;
(d) a first portion comprising the sequence of SEQ ID NO:56; a second portion comprising the sequence of SEQ ID NO:57; and a third portion comprising a sequence of SEQ ID NO: 58;
(e) a first portion comprising the sequence of SEQ ID NO:62; a second portion comprising the sequence of SEQ ID NO:63; and a third portion comprising the sequence of SEQ ID NO: 64, or
the second nucleic acid comprises one or more of the following:
(a) a first portion comprising the sequence of SEQ ID NO:42; a second portion comprising the sequence of SEQ ID NO:43; and a third portion comprising the sequence of SEQ ID NO: 44;
(b) a first portion comprising the sequence of SEQ ID NO:48; a second portion comprising the sequence of SEQ ID NO:49; and a third portion comprising a sequence of SEQ ID NO: 50;
(c) a first portion comprising the sequence of SEQ ID NO:42; a second portion comprising the sequence of SEQ ID NO:54; and a third portion comprising the sequence of SEQ ID NO: 55;
(d) a first portion comprising the sequence of SEQ ID NO:59; a second portion comprising the sequence of SEQ ID NO:60; and a third portion comprising a sequence of SEQ ID NO: 61;
(e) a first portion comprising the sequence of SEQ ID NO:65; a second portion comprising the sequence of SEQ ID NO:66; and a third portion comprising the sequence of SEQ ID NO: 67.

17. The nucleic acid of claim 15, wherein
the first nucleic acid comprises at least one of the following:
(a) a nucleic acid encoding a heavy chain variable region comprising an amino acid sequence of SEQ ID NO:29;
(b) a nucleic acid encoding a heavy chain variable region comprising an amino acid sequence of SEQ ID NO:31;
(c) a nucleic acid encoding a heavy chain variable region comprising an amino acid sequence of SEQ ID NO:33;
(d) a nucleic acid encoding a heavy chain variable region comprising an amino acid sequence of SEQ ID NO:35;
(e) a nucleic acid encoding a heavy chain variable region comprising an amino acid sequence of SEQ ID NO:37;
(f) a nucleic acid encoding a heavy chain variable region comprising an amino acid sequence of SEQ ID NO:87, or
the second nucleic acid comprises at least one of the following:
(a) a nucleic acid encoding a light chain variable region comprising an amino acid sequence of SEQ ID NO:30;
(b) a nucleic acid encoding a light chain variable region comprising an amino acid sequence of SEQ ID NO:32;
(c) a nucleic acid encoding a light chain variable region comprising an amino acid sequence of SEQ ID NO:34;
(d) a nucleic acid encoding a light chain variable region comprising an amino acid sequence of SEQ ID NO:36;
(e) a nucleic acid encoding a light chain variable region comprising an amino acid sequence of SEQ ID NO:38;
(f) a nucleic acid encoding a light chain variable region comprising an amino acid sequence of SEQ ID NO:88.

18. The nucleic acid of claim 15, comprising at least one of the following:
(a) a first nucleic acid comprising a nucleic acid encoding a heavy chain variable region comprising an amino acid sequence of SEQ ID NO:29; and a second nucleic acid comprising a nucleic acid encoding a light chain variable region comprising an amino acid sequence of SEQ ID NO:30;
(b) a first nucleic acid comprising a nucleic acid encoding a heavy chain variable region comprising an amino acid sequence of SEQ ID NO:31; and a second nucleic acid comprising a nucleic acid encoding a light chain variable region comprising an amino acid sequence of SEQ ID NO:32;
(c) a first nucleic acid comprising a nucleic acid encoding a heavy chain variable region comprising an amino acid sequence of SEQ ID NO:33; and a second nucleic acid comprising a nucleic acid encoding a light chain variable region comprising an amino acid sequence of SEQ ID NO:34;
(d) a first nucleic acid comprising a nucleic acid encoding a heavy chain variable region comprising an amino acid sequence of SEQ ID NO:35; and a second nucleic acid comprising a nucleic acid encoding a light chain variable region comprising an amino acid sequence of SEQ ID NO:36;
(e) a first nucleic acid comprising a nucleic acid encoding a heavy chain variable region comprising an amino acid sequence of SEQ ID NO:37; and a second nucleic acid comprising a nucleic acid encoding a light chain variable region comprising an amino acid sequence of SEQ ID NO:38;
(f) a first nucleic acid comprising a nucleic acid encoding a heavy chain variable region comprising an amino acid sequence of SEQ ID NO:87; and a second nucleic acid comprising a nucleic acid encoding a light chain variable region comprising an amino acid sequence of SEQ ID NO:88.

19. The nucleic acid of claim 15, wherein
the first nucleic acid comprises at least one of the following:
(a) a nucleic acid comprising the sequence of SEQ ID NO:68;
(b) a nucleic acid comprising the sequence of SEQ ID NO:70;
(c) a nucleic acid comprising the sequence of SEQ ID NO:72;
(d) a nucleic acid comprising the sequence of SEQ ID NO:74;
(e) a nucleic acid comprising the sequence of SEQ ID NO:76;
(f) a nucleic acid comprising the sequence of SEQ ID NO:89, or
the second nucleic acid comprises at least one of the following:
(a) a nucleic acid comprising the sequence of SEQ ID NO:69;
(b) a nucleic acid comprising the sequence of SEQ ID NO:71;
(c) a nucleic acid comprising the sequence of SEQ ID NO:73;
(d) a nucleic acid comprising the sequence of SEQ ID NO:75;
(e) a nucleic acid comprising the sequence of SEQ ID NO:77;
(f) a nucleic acid comprising the sequence of SEQ ID NO:90.

20. The nucleic acid of claim 15,
comprising at least one of the following:
(a) a first nucleic acid comprising the sequence of SEQ ID NO:68; and a second nucleic acid comprising the sequence of SEQ ID NO:69;
(b) a first nucleic acid comprising the sequence of SEQ ID NO:70; and a second nucleic acid comprising the sequence of SEQ ID NO:71;
(c) a first nucleic acid comprising the sequence of SEQ ID NO:72; and a second nucleic acid comprising the sequence of SEQ ID NO:73;
(d) a first nucleic acid comprising the sequence of SEQ ID NO:74; and a second nucleic acid comprising the sequence of SEQ ID NO:75;
(e) a first nucleic acid comprising the sequence of SEQ ID NO:76; and a second nucleic acid comprising the sequence of SEQ ID NO:77;
(f) a first nucleic acid comprising the sequence of SEQ ID NO:89; and a second nucleic acid comprising the sequence of SEQ ID NO:90.

21. The nucleic acid of claim 15, wherein the nucleic acid comprises an expression vector.

22. An antibody pair, comprising a first antibody and a second antibody, wherein the first antibody and the second antibody each independently comprises:
(a) a heavy chain polypeptide comprising a CDR1 comprising an amino acid sequence of SEQ ID NO:1, a CDR2 comprising an amino acid sequence of SEQ ID NO:2, and a CDR3 comprising an amino acid sequence of SEQ ID NO:3; and a light chain polypeptide comprising CDR1 comprising an amino acid sequence of SEQ ID NO:4, a CDR2 comprising an amino acid sequence of SEQ ID NO:5, and a CDR3 comprising an amino acid sequence of SEQ ID NO:6;
(b) a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9; and a light chain polypeptide comprising CDR1 comprising an amino acid sequence of SEQ ID NO:10, a CDR2 comprising an amino acid sequence of SEQ ID NO:11, and a CDR3 comprising an amino acid sequence of SEQ ID NO:12;
(c) a heavy chain polypeptide comprising a CDR1 comprising an amino acid sequence of SEQ ID NO:13, a CDR2 comprising an amino acid sequence of SEQ ID NO:14, and a CDR3 comprising an amino acid sequence of SEQ ID NO:15; and a light chain polypeptide comprising CDR1 comprising an amino acid sequence of SEQ ID NO:4, a CDR2 comprising an amino acid sequence of SEQ ID NO:16, and a CDR3 comprising an amino acid sequence of SEQ ID NO:17;
(d) a heavy chain polypeptide comprising a CDR1 comprising an amino acid sequence of SEQ ID NO:18, a CDR2 comprising an amino acid sequence of SEQ ID NO:19, and a CDR3 comprising an amino acid sequence of SEQ ID NO:20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22;
(e) a heavy chain polypeptide comprising a CDR1 comprising an amino acid sequence of SEQ ID NO:23, a CDR2 comprising an amino acid sequence of SEQ ID NO:24, and a CDR3 comprising an amino acid sequence of SEQ ID NO:25; and a light chain polypeptide comprising CDR1 comprising an amino acid sequence of SEQ ID NO:26, a CDR2 comprising an amino acid sequence of SEQ ID NO:27, and a CDR3 comprising an amino acid sequence of SEQ ID NO:28, and
wherein the first antibody and the second antibody does not compete for the same epitope on sTNFR2.

23. The antibody pair of claim 22, wherein
the first antibody comprises a heavy chain polypeptide comprising a CDR1 comprising an amino acid sequence of SEQ ID NO:18, a CDR2 comprising an amino acid sequence of SEQ ID NO: 19, and a CDR3 comprising an amino acid sequence of SEQ ID NO:20; and a light chain polypeptide comprising CDR1 comprising an amino acid sequence of SEQ ID NO:4, a CDR2 comprising an amino acid sequence of SEQ ID NO:21, and a CDR3 comprising an amino acid sequence of SEQ ID NO:22, and
the second antibody comprises a heavy chain polypeptide comprising a CDR1 comprising an amino acid sequence of SEQ ID NO:7, a CDR2 comprising an amino acid sequence of SEQ ID NO:8, and a CDR3 comprising an amino acid sequence of SEQ ID NO:9;
and a light chain polypeptide comprising CDR1 comprising an amino acid sequence of SEQ ID NO: 10, a CDR2 comprising an amino acid sequence of SEQ ID NO:11, and a CDR3 comprising an amino acid sequence of SEQ ID NO: 12.

24. The antibody pair claim 22, wherein the first antibody and the second antibody each independently comprises one of the following:
(a) a heavy chain polypeptide comprising a heavy chain variable region comprising an amino acid sequence of SEQ ID NO:29; and a light chain peptide comprising a light chain variable region comprising an amino acid sequence of SEQ ID NO:30;
(b) a heavy chain polypeptide comprising a heavy chain variable region comprising an amino acid sequence of SEQ ID NO:31; and a light chain peptide comprising a light chain variable region comprising an amino acid sequence of SEQ ID NO:32;
(c) a heavy chain polypeptide comprising a heavy chain variable region comprising an amino acid sequence of SEQ ID NO:33; and a light chain peptide comprising a light chain variable region comprising an amino acid sequence of SEQ ID NO:34;
(d) a heavy chain polypeptide comprising a heavy chain variable region comprising an amino acid sequence of SEQ ID NO:35; and a light chain peptide comprising a light chain variable region comprising an amino acid sequence of SEQ ID NO:36;
(e) a heavy chain polypeptide comprising a heavy chain variable region comprising an amino acid sequence of SEQ ID NO:37; and a light chain peptide comprising a light chain variable region comprising an amino acid sequence of SEQ ID NO:38;
(f) a heavy chain polypeptide comprising a heavy chain variable region comprising an amino acid sequence of SEQ ID NO:87; and a light chain peptide comprising a light chain variable region comprising an amino acid sequence of SEQ ID NO:88.

25. The antibody pair claim 22, wherein
the first antibody comprises a heavy chain polypeptide comprising a heavy chain variable region comprising an amino acid sequence of SEQ ID NO:35; and a light chain peptide comprising a light chain variable region comprising an amino acid sequence of SEQ ID NO:36, or the first antibody comprises a heavy chain polypeptide comprising a heavy chain variable region comprising an amino acid sequence of SEQ ID NO:87; and a light chain peptide comprising a light chain variable region comprising an amino acid sequence of SEQ ID NO:88, and
the second antibody comprises a heavy chain polypeptide comprising a heavy chain variable region comprising an amino acid sequence of SEQ ID NO:31; and a light chain peptide comprising a light chain variable region comprising an amino acid sequence of SEQ ID NO:32.

26. The antibody pair claim 22, wherein the antibody pair quantifies sTNFR2 in a sample, and wherein the first antibody captures sTNFR2 in the sample and the second antibody quantifies the sTNFR2 captured by the first antibody.

27. A method of quantifying sTNFR2 in a sample using the antibody pair of claim 22, comprising:
capturing sTNFR2 in the sample with the first antibody; and
quantifying sTNFR2 captured by the first antibody with the second antibody.

28. The method of claim 27, wherein at least one of the following applies:
(a) the method of quantifying is a western blotting-based method, an enzyme-linked immunoassay (ELISA)-based method, or combinations thereof;
(b) the sample is a serum sample, a plasma sample, a urine sample, or combinations thereof;
(c) the sample is a sample from a subject suspected to have a disease or disorder indicated by altered levels of sTNFR2, and the quantification of sTNFR2 in the sample diagnosis the disease or disorder;
(d) the sample is a sample from a subject confirmed to have a disease or disorder involving altered levels of sTNFR2, and wherein the quantification of sTNFR2 predicts outcomes of the disease or disorder or monitors the state of the disease or disorder during therapies.

29-30. (canceled)

31. The method of claim 28, wherein
in (c) the disease is Kawasaki disease (KD) or Kawasaki-like syndrome (KLS), or
in (d) the disease or disorder is lupus nephritis, Behçet's disease, Sjogren's syndrome, rheumatoid arthritis and juvenile chronic arthritis, inflammatory bowel diseases, sarcoidosis, Grave's disease, myocardial infarction, congestive heart failure, progressive diabetic kidney disease in subjects with type 2 diabetes mellitus, erythropoietin resistance in setting of hemodialysis, steatohepatitis, hepatitis C virus liver fibrosis, post-COVID-19 multisystem inflammatory syndrome (MIS-C) in children, presence or prognosis of infections including sepsis, malaria, COVID-19, or HIV.

32-33. (canceled)

34. A method of diagnosing in a subject a disease or disorder involving altered levels of sTNFR2, the method comprising:
capturing sTNFR2 in a sample from the subject with the first antibody of claim 22;
quantifying sTNFR2 captured by the first antibody with the second antibody of claim 22;
wherein, if the sTNFR2 level quantified by the first antibody and the second antibody is equal to or higher than a first predetermined level, the subject is diagnosed as having the disease or disorder, and
wherein, if the sTNFR2 level quantified by the first antibody and the second antibody is lower than a first predetermined level, the subject is diagnosed as not having the disease or disorder.

35. The method of claim 34, wherein at least one of the following applies:
(a) the method of quantifying is a western blotting-based method, an enzyme-linked immunoassay (ELISA)-based method, or combinations thereof;
(b) the sample is a serum sample, a plasma sample, a urine sample, or combinations thereof;
(c) the disease is Kawasaki disease (K_{D}) or Kawasaki-like syndrome (KLS).

36-37. (canceled)

38. A method of monitoring progression and/or state of a disease or disorder involving altered levels of sTNFR2 in a subject diagnosed with the disease or disorder, the method comprising:
obtaining subject's samples at two or more time points after diagnosis of the disease or disorder in the subject;
capturing sTNFR2 in each obtained sample with the first antibody of claim 22;
quantifying sTNFR2 captured by the first antibody with the second antibody of claim 22;
comparing the sTNFR2 levels quantified by the first antibody and the second antibody in the obtained subject's samples;
wherein the comparing step allows for monitoring progression and/or state of the disease or disorder in the subject.

39. The method of claim 38, wherein at least one of the following applies:
(a) at least one of the two or more time points is a time point where the subject is receiving therapeutic treatment for the disease or disorder, and wherein the comparing step allows for monitoring therapeutic outcome of the therapeutic treatment in the subject;
(b) the method of quantifying is a western blotting-based method, an enzyme-linked immunoassay (ELISA)-based method, or combinations thereof;
(c) each sample is independently a serum sample, a plasma sample, a urine sample, or combinations thereof;
(d) the disease or disorder is lupus nephritis, Behçet's disease, Sjogren's syndrome, rheumatoid arthritis and juvenile chronic arthritis, inflammatory bowel diseases, sarcoidosis, Grave's disease, myocardial infarction, congestive heart failure, progressive diabetic kidney disease in subjects with type 2 diabetes mellitus, erythropoietin resistance in setting of hemodialysis, steatohepatitis, hepatitis C virus liver fibrosis, post-COVID-19 multisystem inflammatory syndrome (MIS-C) in children, presence and/or prognosis of infections including sepsis, malaria, COVID-19, or HIV.

40-42. (canceled)

43. A method of treating, ameliorating, and/or preventing Kawasaki disease or Kawasaki-like syndrome in a subject in need thereof, the method comprising:
quantifying sTNFR2 level in a sample from the subject, comprising capturing sTNFR in the sample with the first antibody of claim 16, and quantifying the STNFR2 captured by the first antibody with the second antibody of claim 16;
administering to the subject an effective amount of a medication for treating Kawasaki disease or Kawasaki-like syndrome if the sTNFR2 level quantified by the first antibody and the second antibody is equal to or higher than a first predetermined level;
optionally wherein the sample is a serum sample, a plasma sample, a urine sample, or combinations thereof;
optionally wherein the medication comprises an intravenous immunoglobulin (IVIG), aspirin, a steroid, or combinations thereof.

44. (canceled)

45. The method of claim 43, wherein the method further comprises:
quantifying C—C motif chemokine ligand 1 (Ccl1) level in the sample;
quantifying C—C motif chemokine ligand 2 (Ccl2) level in the sample; and
quantifying C—X—C motif chemokine 11 (Cxc11) level in the sample, and
wherein the subject is administered with the medication for treating Kawasaki disease or Kawasaki-like syndrome if the sTNFR2 level quantified by the first antibody and the second antibody is equal to or higher than a first predetermined level, and if at least two of the following are true:
(a) the quantified Ccl1 level is higher than a second predetermined level,
(b) the quantified Ccl2 level is higher than a third predetermined level,
(c) the quantified Cxc11 level is higher than a fourth predetermined level.

46. (canceled)

47. A method of treating, ameliorating and/or preventing an inflammatory disease or disorder in a subject in need thereof, wherein the method comprises:
administering to the subject an effective amount of an antibody of claim 1,
wherein the inflammatory disease is caused by or involves overactive TNFα-TNFR2 signaling.

48. The method of claim 47, wherein at least one of the following applies:
(a) the disease or disorder is at least one selected from the group consisting of lupus nephritis, plaque psoriasis, psoriatic arthritis, ankylosing spondylitis, Crohn's disease, ulcerative colitis, hidradenitis, uveitis, Kawasaki disease, Behçet's disease, Sjogren's syndrome, rheumatoid arthritis and juvenile chronic arthritis, juvenile rheumatoid arthritis with macrophage activation syndrome, inflammatory bowel diseases, sarcoidosis, Grave's disease, myocardial infarction, congestive heart failure, progressive diabetic kidney disease in subjects with type 2 diabetes mellitus, steatohepatitis, hepatitis C virus liver fibrosis, post-COVID-19 multisystem inflammatory syndrome (MIS-C) in children and adults (MIS-A), bacterial sepsis, interstitial lung disease, cerebral malaria, acute respiratory distress syndrome (ARDS) or diffuse alveolar hemorrhage (DAH) associated with viral infections comprising COVID-19, and bacterial infections comprising leptospirosis;
(b) the method reduces a level of interferon γ (IFN-γ) in the subject;
(c) the method reduces an expression of IFN-γ by the peripheral blood mononuclear cells (PBMCs);
(d) the subject is a mammal, optionally a human.

49-51. (canceled)


SEQUENCE LISTING (90 sequences)

SEQ ID NO: 1 - protein, 7 aa, synthetic construct
GFTFSTA

SEQ ID NO: 2 - protein, 10 aa, synthetic construct
QIKAKSNNYA

SEQ ID NO: 3 - protein, 10 aa, synthetic construct
YCTGGNSVDY

SEQ ID NO: 4 - protein, 6 aa, synthetic construct
QNINKF

SEQ ID NO: 5 - skipped

SEQ ID NO: 6 - protein, 9 aa, synthetic construct
LQHIRFPGT

SEQ ID NO: 7 - protein, 7 aa, synthetic construct
GYTFTDH

SEQ ID NO: 8 - protein, 8 aa, synthetic construct
EISPNYDT

SEQ ID NO: 9 - protein, 11 aa, synthetic construct
YCSRTNSGWYF

SEQ ID NO: 10 - protein, 5 aa, synthetic construct
SSVRN

SEQ ID NO: 11 - skipped

SEQ ID NO: 12 - protein, 9 aa, synthetic construct
QQWSRNPPT

SEQ ID NO: 13 - protein, 7 aa, synthetic construct
GFTFSNA

SEQ ID NO: 14 - protein, 10 aa, synthetic construct
QIKAKSNNFA

SEQ ID NO: 15 - protein, 10 aa, synthetic construct
FCAGGNTWDY

SEQ ID NO: 16 - skipped

SEQ ID NO: 17 - protein, 9 aa, synthetic construct
LQHASFPGT

SEQ ID NO: 18 - protein, 9 aa, synthetic construct
GFSLSTYGV

SEQ ID NO: 19 - protein, 7 aa, synthetic construct
NIWWDDD

SEQ ID NO: 20 - protein, 11 aa, synthetic construct
YCARIDGTRYF

SEQ ID NO: 21 - skipped

SEQ ID NO: 22 - protein, 8 aa, synthetic construct
LQYGNLWT

SEQ ID NO: 23 - protein, 7 aa, synthetic construct
GYIFTSY

SEQ ID NO: 24 - protein, 8 aa, synthetic construct
YINTGNGG

SEQ ID NO: 25 - protein, 13 aa, synthetic construct
YCARGHPYYGYYL

SEQ ID NO: 26 - protein, 6 aa, synthetic construct
QDIGNW

SEQ ID NO: 27 - skipped

SEQ ID NO: 28 - protein, 9 aa, synthetic construct
LQTYTSPWT

SEQ ID NO: 29 - protein, 117 aa, synthetic construct
QVQLKESGGSLVQPGKSLKLTCVTSGFTFSTAWMHWVRQSPEKQLEWVAQIKAKSNNYATYYAESVKGRFTISRDDSKNSVYLQMNSLKEEDTAIYYCTGGNSVDYWGQGVMVTVSS

SEQ ID NO: 30 - protein, 107 aa, synthetic construct
NIVLTQSPSFLPASVGDRVTINCKASQNINKFLNWYQQKLGEAPKRLIYNTNNLDTGTPSRFSGSGSGTDYTLTISSLQPEDFATYFCLQHIRFPGTFGGGTMLELR

SEQ ID NO: 31 - protein, 118 aa, synthetic construct
QVQLKESGAALVKPGASVKMSCKASGYTFTDHWVSWVRQSHGRSLEWIGEISPNYDTTNFNEKFRGRATLTVDKSTSTASMELSRLTSEDSAIYYCSRTNSGWYFDFWGPGTMVTVSS

SEQ ID NO: 32 - protein, 106 aa, synthetic construct
NIVMTQSPTTTAASPGEKVTITCLASSSVRNIYWYQQKSGASPKPLIYSTSSLASGVPDRFSGSGSGTSYSLTINTMEAEDAATYYCQQWSRNPPTFGSGTKLEIK

SEQ ID NO: 33 - protein, 117 aa, synthetic construct
QVQLKESGGSLVQPGKSLKLTCATSGFTFSNAWMHWVRQSPEKQLEWVAQIKAKSNNFATYYAESVRGRFIISRDDSKSTVYLQMNNLREEDTAIYFCAGGNTWDYWGQGVMVTVSS

SEQ ID NO: 34 - protein, 107 aa, synthetic construct
AAANSPLIGAPGGAEGDRVTINCTASQNINKFLNWYQQRLGKAPKRLILSPNNLQAGVPSRFSGSGSGTDYTLTISNLQPEDFASYFCLQHASFPGTFGGGTKLELI

SEQ ID NO: 35 - protein, 119 aa, synthetic construct
QVQLKESGPGILQPSQTLSLTCTFSGFSLSTYGVGVGWIRQPSGKGLEWLANIWWDDDKYYNPSLKNRLTISKDTSNNQVFLKITNVDTADTATYYCARIDGTRYFDYWGQGVMVSVSS

SEQ ID NO: 36 - protein, 106 aa, synthetic construct
DIVLTQSPPSLSASLGDKVTITCQTSQNINKFIAWYQQKPGKAPRQLIRYTSTLMSGTPSRFSGSGSGTDYSFSISNVESEDIATYYCLQYGNLWTFGRGTKLELK

SEQ ID NO: 37 - protein, 120 aa, synthetic construct
QVQLTESGAELAKPGSSVKISCKASGYIFTSYYISWIKQTTGQGLDYVGYINTGNGGTHYSERFKDSATLTVDRSSSTAFMQLSSLTPDDSAVYYCARGHPYYGYYLDYWGQGVMVTVSS

SEQ ID NO: 38 - protein, 107 aa, synthetic construct
DIVLTQSPASLSASLEEIVTITCQASQDIGNWLSWYQQSPGKSPQLLIYGATSLADGVPSRFSGSRSGTQFSLTISRLQVEDIGIYYCLQTYTSPWTFGGGTKLELK

SEQ ID NO: 39 - DNA, 21 bp, synthetic construct
  source 1..21: mol_type = other DNA
ggattcactttcagtaccgcc

SEQ ID NO: 40 - DNA, 30 bp, synthetic construct
  source 1..30: mol_type = other DNA
caaattaaagccaaatcaaacaattatgca

SEQ ID NO: 41 - DNA, 30 bp, synthetic construct
  source 1..30: mol_type = other DNA
tattgtacgggagggaatagtgttgattac

SEQ ID NO: 42 - DNA, 18 bp, synthetic construct
  source 1..18: mol_type = other DNA
cagaatattaacaagttc

SEQ ID NO: 43 - skipped

SEQ ID NO: 44 - DNA, 27 bp, synthetic construct
  source 1..27: mol_type = other DNA
ttacagcatattcgttttcccgggacg

SEQ ID NO: 45 - DNA, 21 bp, synthetic construct
  source 1..21: mol_type = other DNA
ggttatacattcactgaccac

SEQ ID NO: 46 - DNA, 24 bp, synthetic construct
  source 1..24: mol_type = other DNA
gaaatatctcctaactacgatact

SEQ ID NO: 47 - DNA, 33 bp, synthetic construct
  source 1..33: mol_type = other DNA
tactgttcaagaacgaactcaggctggtacttt

SEQ ID NO: 48 - DNA, 15 bp, synthetic construct
  source 1..15: mol_type = other DNA
tccagtgtaaggaac

SEQ ID NO: 49 - skipped

SEQ ID NO: 50 - DNA, 27 bp, synthetic construct
  source 1..27: mol_type = other DNA
cagcagtggagtaggaacccacctacg

SEQ ID NO: 51 - DNA, 21 bp, synthetic construct
  source 1..21: mol_type = other DNA
ggattcactttcagtaacgcc

SEQ ID NO: 52 - DNA, 30 bp, synthetic construct
  source 1..30: mol_type = other DNA
caaattaaagccaaatcaaacaattttgca

SEQ ID NO: 53 - DNA, 30 bp, synthetic construct
  source 1..30: mol_type = other DNA
ttctgtgccggcggtaacacctgggattac

SEQ ID NO: 54 - skipped

SEQ ID NO: 55 - DNA, 27 bp, synthetic construct
  source 1..27: mol_type = other DNA
ttgcagcatgctagttttcccgggacg

SEQ ID NO: 56 - DNA, 30 bp, synthetic construct
  source 1..30: mol_type = other DNA
tctgggttttcactgagcacttatggtgtg

SEQ ID NO: 57 - DNA, 21 bp, synthetic construct
  source 1..21: mol_type = other DNA
aacatttggtgggatgatgat

SEQ ID NO: 58 - DNA, 33 bp, synthetic construct
  source 1..33: mol_type = other DNA
tactgtgctcggatagatggtacccggtacttt

SEQ ID NO: 59 - DNA, 18 bp, synthetic construct
  source 1..18: mol_type = other DNA
caaaacattaacaagttt

SEQ ID NO: 60 - skipped

SEQ ID NO: 61 - DNA, 24 bp, synthetic construct
  source 1..24: mol_type = other DNA
ctacagtacggtaacctgtggacg

SEQ ID NO: 62 - DNA, 21 bp, synthetic construct
  source 1..21: mol_type = other DNA
ggctacattttcaccagctac

SEQ ID NO: 63 - DNA, 24 bp, synthetic construct
  source 1..24: mol_type = other DNA
tatattaatacgggaaatggaggt

SEQ ID NO: 64 - DNA, 39 bp, synthetic construct
  source 1..39: mol_type = other DNA
tactgtgcaagaggccatccatactatggatattacctt

SEQ ID NO: 65 - DNA, 18 bp, synthetic construct
  source 1..18: mol_type = other DNA
caggacatcggtaattgg

SEQ ID NO: 66 - skipped

SEQ ID NO: 67 - DNA, 27 bp, synthetic construct
  source 1..27: mol_type = other DNA
ctacagacttatactagtccgtggacg

SEQ ID NO: 68 - DNA, 352 bp, synthetic construct
  source 1..352: mol_type = other DNA
caggtgcaactaaaagagtcagggggaagcttggtgcagcctgggaaatctctaaaactcacctgtgtcacctcaggattcactttcagtaccgcctggatgcactgggttcgccagtctccagaaaaacaactcgagtgggttgctcaaattaaagccaaatcaaacaattatgcaacctactatgcggagtctgttaaaggaagattcaccatctcaagagatgattccaaaaacagcgtttatttgcagatgaacagcttaaaagaggaagacactgccatttattattgtacgggagggaatagtgttgattactggggccaaggagtcatggtcacagtctcctcag

SEQ ID NO: 69 - DNA, 322 bp, synthetic construct
  source 1..322: mol_type = other DNA
aacattgtgctgactcagtctccttcattcctgcctgcatctgtgggagacagagtcactatcaactgcaaagcaagtcagaatattaacaagttcttaaactggtatcagcaaaagcttggagaggcccccaaacgcctgatatataatacaaacaatttggacacaggcaccccatcaaggttcagtggcagtgggtctggtacagattacacactcaccatcagcagcctgcagcctgaagattttgccacatatttctgcttacagcatattcgttttcccgggacgttcggtggaggcaccatgttggaattgagac

SEQ ID NO: 70 - DNA, 355 bp, synthetic construct
  source 1..355: mol_type = other DNA
caggtacaactaaaggagtcaggggctgcactggtgaagcctggagcctctgtgaagatgtcttgcaaggcttctggttatacattcactgaccactgggtgagctgggtgagacagagtcatggaaggagccttgagtggattggggaaatatctcctaactacgatactactaacttcaatgaaaagttcaggggcagggccacattgactgtcgacaaatccaccagcacagcctctatggagctcagcagattgacatctgaggactctgcaatctactactgttcaagaacgaactcaggctggtactttgacttctggggcccaggaaccatggtcaccgtgtcttcag

SEQ ID NO: 71 - DNA, 319 bp, synthetic construct
  source 1..319: mol_type = other DNA
aacattgtcatgactcagtctccaacaaccacggctgcatctccaggggagaaggtgaccatcacctgccttgccagctccagtgtaaggaacatatactggtaccagcagaagtcaggcgcttcccccaaacccttgatttattccacatcgagcttggcttccggagtcccagatcgcttcagtggcagtgggtctgggacctcttattctctcacaatcaacaccatggaggctgaagatgctgccacttattactgtcagcagtggagtaggaacccacctacgttcggttctgggaccaagttggagatcaaac

SEQ ID NO: 72 - DNA, 352 bp, synthetic construct
  source 1..352: mol_type = other DNA
caggtgcagctaaaggagtcagggggaagcttggtgcagcctgggaaatctctaaaactcacctgtgccacctcaggattcactttcagtaacgcctggatgcactgggttcgccagtctccagagaaacaactagagtgggttgctcaaattaaagccaaatcaaacaattttgcaacctactatgcggagtctgtgagaggaagattcatcatctcgagagatgattccaaaagcaccgtttacctgcagatgaacaacttaagagaggaagacactgccatttatttctgtgccggcggtaacacctgggattactggggccaaggagtcatggtcacagtctcctcag

SEQ ID NO: 73 - DNA, 322 bp, synthetic construct
  source 1..322: mol_type = other DNA
gcggccgcgaattcgcccttgatcggcgcgccaggtggagctgaaggagacagagtcactatcaactgcacagcaagtcagaatattaacaagttcttaaactggtatcagcaaagacttggaaaagctcccaaacgcctgatattgagtccaaacaatttgcaagcaggcgtcccatcaaggttcagtggcagtggatctggtacagattacacactcaccatcagcaacctgcagcctgaagactttgcctcatatttctgcttgcagcatgctagttttcccgggacgttcggtggaggcaccaagctggaattgatcc

SEQ ID NO: 74 - DNA, 358 bp, synthetic construct
  source 1..358: mol_type = other DNA
caggtgcagctgaaagagtcaggccctgggatattgcagccctcccagaccctcagtctgacttgcactttctctgggttttcactgagcacttatggtgtgggtgtgggctggattcgtcagccttcagggaagggtctggagtggctggcaaacatttggtgggatgatgataagtactacaatccatctctgaaaaaccggctcacaatctccaaggacacctccaacaatcaagtgttcctcaagatcaccaatgtggacactgcagatactgccacatactactgtgctcggatagatggtacccggtactttgattactggggtcaaggagtcatggtctcagtctcctcag

SEQ ID NO: 75 - DNA, 319 bp, synthetic construct
  source 1..319: mol_type = other DNA
gacattgtgttgactcagtctcctccctccctgtctgcatctctgggagacaaggtcaccatcacttgccagacaagtcaaaacattaacaagtttatagcttggtatcagcaaaagcctggaaaagctcctaggcagctcatacgttacacttctacactgatgtcaggtaccccatcgaggttcagtggcagtggatctgggacagattattcattcagcatcagcaacgtggagtctgaagatattgcaacttattattgtctacagtacggtaacctgtggacgttcggtagaggcaccaagctggaattgaagc

SEQ ID NO: 76 - DNA, 361 bp, synthetic construct
  source 1..361: mol_type = other DNA
caggtacaactaacggagtcaggagctgaactggcaaagcctggctcttcagtgaagatttcctgcaaggcttctggctacattttcaccagctactatataagctggataaaacagacgactggacagggccttgactatgttggatatattaatacgggaaatggaggtactcactacagtgagaggttcaaggacagcgccacattgactgtagacagatcctccagcacagccttcatgcaactcagcagcctgacacctgacgactctgcggtctattactgtgcaagaggccatccatactatggatattaccttgattactggggccaaggagtcatggtcacagtctcctcag

SEQ ID NO: 77 - DNA, 322 bp, synthetic construct
  source 1..322: mol_type = other DNA
gacattgtcttgactcagtctcctgcctccctgtctgcatctctggaggaaattgtcaccatcacatgccaggcaagccaggacatcggtaattggttatcatggtatcagcagagcccagggaaatctcctcagctcctgatctatggtgcaaccagcttggcagatggggtcccatcaaggttcagcggcagtagatctggcacacagttttctcttacaatcagcagactgcaggttgaagatattggaatctactactgtctacagacttatactagtccgtggacgttcggtggaggcaccaaactggaactgaaac

SEQ ID NO: 78 - DNA, 33 bp, synthetic construct
  source 1..33: mol_type = other DNA
gatatctgtgttgcccgcccaggtggcatttac

SEQ ID NO: 79 - DNA, 31 bp, synthetic construct
  source 1..31: mol_type = other DNA
gatatcgcgtcgccagtgctcccttcagctg

SEQ ID NO: 80 - protein, 493 aa, synthetic construct
MYRMQLLSCIALSLALVTNSISVLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDAISAMVRSPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 81 - protein, 493 aa, synthetic construct
MAPVAVWAALAVGLELWAAAHAVLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDAISAMVRSPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 82 - protein, 446 aa, synthetic construct
QVQLKESGPGILQPSQTLSLTCTFSGFSLSTYGVGVGWIRQPSGKGLEWLANIWWDDDKYYNPSLKNRLTISKDTSNNQVFLKITNVDTADTATYYCARIDGTRYFDYWGQGVMVSVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 213 aa, synthetic construct
DIVLTQSPPSLSASLGDKVTITCQTSQNINKFIAWYQQKPGKAPRQLIRYTSTLMSGTPSRFSGSGSGTDYSFSISNVESEDIATYYCLQYGNLWTFGRGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - DNA, 639 bp, synthetic construct
  source 1..639: mol_type = other DNA
gacatcgtgctgacacagagcccccctagcctgagcgcaagcctgggcgacaaggtgaccatcacctgtcagacatctcagaacatcaacaagttcatcgcctggtatcagcagaagcccggcaaggcccctagacagctgatcagatacacaagcaccctgatgagcggcacccctagcagattcagcggcagcggcagcggcaccgactacagcttcagcatcagcaacgtggagagcgaggacatcgccacctactactgcctgcagtacggcaacctgtggaccttcggcagaggcaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 85 - protein, 446 aa, synthetic construct
QVQLKESGPGILQPSQTLSLTCTFSGFSLSTYGVGVGWIRQPSGKGLEWLANIWWDDDKYYNPSLKNRLTISKDTSNNQVFLKITNVDTADTATYYCARIDGTRYFDYWGQGVMVSVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 86 - DNA, 1338 bp, synthetic construct
  source 1..1338: mol_type = other DNA
caagtgcagctgaaggagagcggccccggcatcctgcagccttctcagaccctgagcctgacctgcaccttcagcggcttcagcctgagcacctacggcgtgggcgtgggctggatcagacagcctagcggcaagggcctggagtggctggccaacatctggtgggacgacgacaagtactacaaccctagcctgaagaacagactgacgattagcaaggacacaagcaacaaccaagtgttcctgaagatcaccaacgtggacaccgccgacaccgccacctactactgcgctagaatcgacggcacaagatacttcgactactggggccaaggcgtgatggtgagcgtgagcagcgcaagcaccaagggtcctagcgtgttccctttagccccctgcagcagaagcacaagcgagagcaccgccgccctgggctgcctagttaaggactacttccctgagcccgtgacggtctcctggaacagtggtgcgctcacaagcggcgtgcacaccttccccgccgtgctgcagagcagcggcctgtacagcctgagcagcgtggtgaccgtccctagcagcagcctgggcaccaagacctacacctgcaacgtggaccacaagcctagcaacaccaaggtggacaaaagagttgagagtaaatacggcccgccttgtcctccttgcccggcccccgaagccgccggaggccctagcgttttcctgttcccccccaagcccaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgccgagcagcatcgaaaagacaataagcaaggccaaagggcagcctagagagccccaagtgtacaccctgccccctagccaagaggagatgaccaagaaccaagtgagcctgacttgcctcgtgaagggattctaccctagcgacatcgccgtggagtgggagagcaacgggcagcctgagaacaactacaagaccaccccccccgtgctggacagcgacggcagcttcttcctgtacagcaagctgaccgtggacaagagcagatggcaagagggcaacgtgttcagctgcagcgtgatgcacgaggccctgcacaaccactacacacagaagagcctgagcctgagcctgggcaag

SEQ ID NO: 87 - protein, 119 aa, synthetic construct; identical to SEQ ID NO: 35

SEQ ID NO: 88 - protein, 106 aa, synthetic construct
DIVLTQSPPSLSASLGDKVTITCQTSQNINKFIAWYQQKPGKAPRQLIRYTSTLMSGTPSRFSGSGSGTDYSFSISNVESEDIATYYCLQYGNLWTFGRGTKLEIK

SEQ ID NO: 89 - DNA, 357 bp, synthetic construct
  source 1..357: mol_type = other DNA
caagtgcagctgaaggagagcggccccggcatcctgcagccttctcagaccctgagcctgacctgcaccttcagcggcttcagcctgagcacctacggcgtgggcgtgggctggatcagacagcctagcggcaagggcctggagtggctggccaacatctggtgggacgacgacaagtactacaaccctagcctgaagaacagactgacgattagcaaggacacaagcaacaaccaagtgttcctgaagatcaccaacgtggacaccgccgacaccgccacctactactgcgctagaatcgacggcacaagatacttcgactactggggccaaggcgtgatggtgagcgtgagcagc

SEQ ID NO: 90 - DNA, 318 bp, synthetic construct
  source 1..318: mol_type = other DNA
gacatcgtgctgacacagagcccccctagcctgagcgcaagcctgggcgacaaggtgaccatcacctgtcagacatctcagaacatcaacaagttcatcgcctggtatcagcagaagcccggcaaggcccctagacagctgatcagatacacaagcaccctgatgagcggcacccctagcagattcagcggcagcggcagcggcaccgactacagcttcagcatcagcaacgtggagagcgaggacatcgccacctactactgcctgcagtacggcaacctgtggaccttcggcagaggcaccaagctcgagatcaag